(19) United States
(12) Patent Application Publication
(10) Pub. No.: US 2025/0179033 A1
(43) Pub. Date: Jun. 5, 2025

(54) NEW ANTIVIRAL TRIAZOLE DERIVATIVES, THEIR SYNTHESIS AND THEIR USE FOR TREATMENT OF MAMMALIAN VIRAL INFECTIONS

(71) Applicants: Collaborations Pharmaceuticals, Inc., Fuquay Varina, NC (US); Federal Research Center Fundamentals of Biotechnology Russian Academy of Science, Moscow (RU)

(72) Inventors: Vadim Makarov, Moscow (RU); Olga Riabova, Moscow (RU); Thomas R. Lane, Durham, NC (US); Sean Ekins, Fuquay Varina, NC (US)

(21) Appl. No.: 18/725,926

(22) PCT Filed: Dec. 30, 2021
(86) PCT No.: PCT/RU2021/000624

§ 371 Date: Jul. 1, 2024

(51) Int. Cl. C07D 249/14; A61K 31/4196; A61K 31/4245; A61K 31/427; A61K 31/429; A61K 31/4439; A61K 31/4709; A61K 31/506; A61P 31/18; C07D 401/12; C07D 401/14; C07D 403/12; C07D 403/14; C07D 409/12; C07D 413/12; C07D 417/12; C07D 417/14; C07D 513/04

(52) U.S. Cl. CPC C07D 249/14; A61K 31/4196; A61K 31/4245; A61K 31/427; A61K 31/429; A61K 31/4439; A61K 31/4709; A61K 31/506; A61P 31/18; C07D 401/12; C07D 401/14; C07D 403/12; C07D 403/14; C07D 409/12; C07D 413/12; C07D 417/12; C07D 417/14; C07D 513/04

(57) ABSTRACT

A family of triazole derivatives is described. Also described is the use of these triazole derivatives in treating or preventing viral infections, such as human immunodeficiency virus (HIV) infections, and the diseases caused by these infections, such as acquired immunodeficiency syndrome (AIDS). The triazole derivatives, for example, comprise a central triazole group with two substituents comprising an aryl or heteroaryl group. Exemplary derivatives showed excellent HIV inhibitory activity against both wild-type and selected mutant strains of HIV.

### GOVERNMENT INTEREST

This invention was made with government support under Grant Number 1R01NS102164-01 awarded by the National Institutes of Health. The government has certain rights in the invention.

This invention was made with government support under Grant Number 17-54-30007 awarded by Russian Foundation of Fundamental Research.

### TECHNICAL FIELD

The presently disclosed subject matter relates to a novel family of triazole derivatives and their use as antiviral agents in treating or preventing viral infections and/or related diseases in humans and other animals. Exemplary infections and diseases treatable or preventable by the antiviral agents include human immunodeficiency virus (HIV) infections and acquired immunodeficiency syndrome (AIDS).

### BACKGROUND

Human immunodeficiency virus (HIV) infection, the virus that causes acquired immunodeficiency syndrome (AIDS), is an important pathogen that can affect mankind. The World Health Organization (WHO) reports that HIV has claimed more than 32.7 million (24.8-42.2 million) lives globally since the start of the epidemic, with approximately 690,000 people dying from HIV-related illnesses in 2019 alone. In 2019, the number of new HIV infections was about 1.7 million, while the number of people living with HIV was about 38.0 million (1.7 million of whom were children under 15 years or age, with 150,000 newly infected in 2019) (1, 2). HIV is prevalent in the developing world, but it is also resurfacing in wealthy countries, with some 37,968 cases reported in the US in 2018 according to the Centers for Disease Control and Prevention (CDC) and over 1 million currently live with HIV in the United States of America (1, 3). And yet, there is reason for cautious optimism that HIV can be managed, albeit with a cocktail of 3-4 antiretroviral drugs that need to be taken regularly.

The implementation of combination antiretroviral therapy (cART) or the synonymous highly active antiretroviral therapy (HAART) can delay the progression of the most severe symptoms of HIV for decades by restoring the immune system and controlling viral load (4). cART/HAART is a cocktail of multiple HIV-targeting drugs, with the most common regimens being comprised of a nucleoside reverse transcriptase inhibitor (NRTI), a non-nucleoside reverse transcriptase inhibitor (NNRTI), and/or protease inhibitors (5). cART/HAART is a common intervention in HIV-infected children as well, with approximately 54% of children living with HIV receiving cART/HAART in 2018 globally (1). Early cART/HAART in children improves their immune reconstitution, drastically reducing AIDS-related mortality (6, 7).

However, while cART/HAART has overall been quite effective, low-level viremia (LLV) is common even among those undergoing cART/HAART, though this prevalence is variable between populations (0.4%-38.7%) (8-11) and with the added complexity that there is no standard definition of LLV. LLV has clinical relevance due to it leading to an increased risk of virological failure, transmission, and for the development of antiviral resistance (12). This is exacerbated in individuals with persistent LLV, particularly with regard to an increased risk of virological failure (13, 14). Some recent publications have further analyzed the relationship between LLV and/or virological failure with the appearance of drug-resistant mutants in 42.6% (Northern Taiwan), 70.2% (Cameroon) and 92% (Northern Tanzania) of patients with either LLV or virological failure. These clinical isolates were found to have at least one drug resistant mutation (15-17). Many of these mutations had resistance to NRTI(s) and/or NNRTI(s). Mutations imbuing resistance against NNRTIs were the most common, with K103N occurring with the highest frequency, followed by Y181C. These are common clinical HIV-RT mutations that are resistant to established drugs, with K103N, Y181C, and G190A accounting for more than 90% of NNRTI resistance in the United States (18)).

Side effects are also a concern for HIV drugs, particularly as these drugs often need to be taken for decades since HIV is now treated as a chronic disease (19). In addition, a large number of patients develop HIV associated neurological disorders (HAND), which can result in symptoms from minor problems with memory to severe dementia-like symptoms. Autopsy studies have been reported showing white matter changes and demyelination (20). Even though small molecule treatments are highly effective in the periphery, virus can remain in the CNS and replicate, which can then result in the neurological disorders. It is believed possible that this outcome is attributable to the inability of HIV medications to cross the blood brain barrier (BBB) (21) and inhibit HIV in the brain. Several reasons have been suggested for why combination antiretroviral therapy (cART) is inefficient at preventing HAND, such as poor central nervous system (CNS) penetration and incomplete inhibition of HIV replication in this anatomical compartment, drug resistance, cART neurotoxicity, or irreversible brain damage prior to initiation of cART. So far, the data is mixed on CNS-targeted ART (22, 23) and there have been several studies assessing cerebrospinal fluid (CSF) concentrations of drugs and viral suppression in adults (24) and children (25) showing adequate viral suppression for some drugs and suboptimal CSF concentrations for others. High CNS penetration of ART is also important to limit tissue injury and recovery in those at risk of cerebrovascular disease (26). Some drugs, such as efavirenz, are well documented from the perspective of CNS effects, which can be due to multiple mechanisms (27). Macrophages, particularly in the CNS, are likely components of the persistent reservoir that resist HIV eradication. However, cART has limited effect in macrophages, due to their scarce phosphorylation activity, which limits the activity of nucleoside analogs, and the expression of P-gp transporters (28), which pump out protease inhibitors. There are also issues relating to drug-drug interactions between HIV treatments (due to P450's (29)) or CNS side effects (27, 30-35). It would therefore be helpful to optimize novel NNRTI that cross the BBB (19) and that are effective at preventing viral replication in the brain and the periphery. New anti-HIV compounds, selected specifically for their ability to overcome the growing list of HIV RT strains (18) that are resistant to established drugs, are beginning to populate a small pipeline of potential future drugs (18, 36, 37).

Accordingly, there is an ongoing need to provide additional antiviral agents, e.g., for the treatment and/or prevention of HIV infection. In particular, the is a need for additional agents that can address both drug resistance and HIV CNS dysfunctions.

### SUMMARY

In some embodiments, the presently disclosed subject matter provides a compound having a structure of Formula (I):

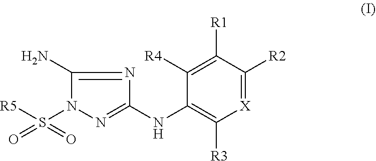

wherein R1, R2, R3, and R4 are independently selected from the group comprising H, CN, CH₂CN, CH═CHCN, F, Cl, and lower alkyl; X is CR6 or N, wherein R6 is selected from the group comprising H, CN, CH₂CN, CH═CHCN, COCH₃, C(CH₃)═CHCN, F, Cl, Br, lower alkyl, MeO, NMe₂, 4-F-Ph, and NMeCOCH═CH₂; and R5 is a monovalent aryl group selected from the group comprising:

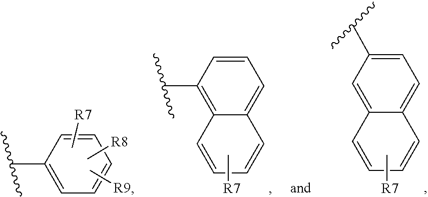

wherein R7, R8, and R9 are independently selected from the group comprising H, CN, CH₂CN, CH═CHCN, COCH₃, C(CH₃)═CHCN, F, Cl, Br, lower alkyl, MeO, NMe₂, 4-F-Ph, and NMeCOCH═CH₂; or wherein R5 is a heteroaryl radical selected from the group comprising:

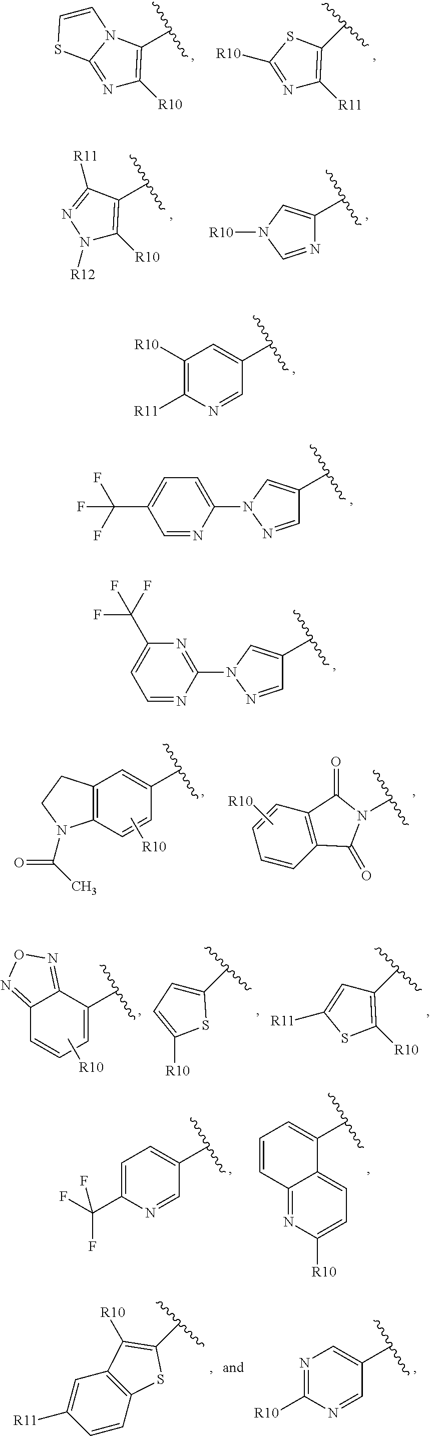

wherein R10, R11, and R12 are independently selected from the group comprising H, F, Cl, Br, CN, lower alkyl, MeO, CF₃, and NHCOCH₃; or a pharmaceutically acceptable salt thereof.

In some embodiments, R5 is

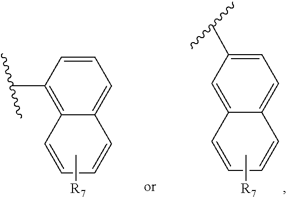

where R7 is selected from the group comprising H, CN, CH₂CN, CH═CHCN, COCH₃, C(CH₃)═CHCN, F, Cl, Br, lower alkyl, MeO, NMe₂, 4-F-Ph, and NMeCOCH═CH₂. In some embodiments, R2 is selected from CN, CH₂CN, CH═CHCN, F, Cl, and lower alkyl.

In some embodiments, R5 is:

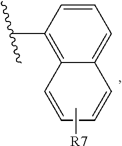

X is CR6, and R4 is H, and the compound of Formula (I) has a structure of:

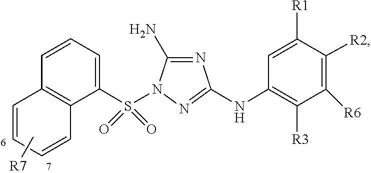

wherein R1, R2, and R3 are independently selected from the group comprising H, CN, CH₂CN, CH═CHCN, F, Cl, and lower alkyl; R6 is selected from the group comprising H, CN, CH₂CN, CH═CHCN, COCH₃, C(CH₃)=CHCN, F, Cl, Br, lower alkyl, MeO, NMe₂, 4-F-Ph, and NMeCOCH═CH₂; and R7 is selected from the group comprising H, CN, CH₂CN, CH═CHCN, COCH₃, C(CH₃)=CHCN, F, Cl, Br, lower alkyl, MeO, NMe₂, 4-F-Ph, and NMeCOCH═CH₂; optionally wherein R7 is attached to carbon 6 or carbon 7.

In some embodiments, R1 and R3 are each H. In some embodiments, R2 is selected from CN, CH₂CN, and CH═CHCN and R6 is selected from H, CN, and Cl; optionally wherein R2 is CN and R6 is Cl, R2 is CN and R6 is CN, or R2 is CH₂CN and R6 is H. In some embodiments, R7 is CH═CHCN.

In some embodiments, R5 is

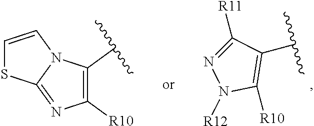

wherein R10, R11, and R12 are independently selected from the group comprising H, F, Cl, Br, CN, lower alkyl, MeO, CF₃, and NHCOCH₃, optionally wherein R11 and R12 are each lower alkyl and R10 is selected from H and Cl.

In some embodiments, X is CR6 and the compound has a structure of:

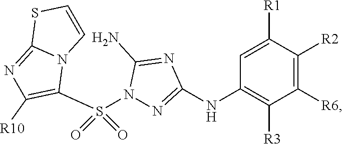

wherein R1, R2, and R3 are independently selected from the group comprising H, CN, CH₂CN, CH═CHCN, F, Cl, and lower alkyl; R6 is selected from the group comprising H, CN, CH₂CN, CH═CHCN, COCH₃, C(CH₃)=CHCN, F, Cl, Br, lower alkyl, MeO, NMe₂, 4-F-Ph, and NMeCOCH═CH₂; and R10 is selected from the group comprising H, F, Cl, Br, CN, lower alkyl, MeO, CF₃, and NHCOCH₃, optionally wherein R10 is selected from H and Cl. In some embodiments, R1 and R3 are each H; R2 is selected from CN, CH₂CN, and CH═CHCN; and R6 is selected from H, CN, and Cl; optionally wherein R10 is Cl, further optionally wherein R2 is CN and R6 is H.

In some embodiments, the compound is selected from the group comprising: 5-[[5-amino-3-(4-cyanoanilino)-1,2,4-triazol-1-yl]sulfonyl]naphthalene-2-carbonitrile; 5-[[5-amino-3-(3-chloro-4-cyano-anilino)-1,2,4-triazol-1-yl]sulfonyl]naphthalene-2-carbonitrile; 4-[[5-amino-1-(6-chloroimidazo[2,1-b]thiazol-5-yl)sulfonyl-1,2,4-triazol-3-yl]amino]-2-chloro-benzonitrile; 4-[[5-amino-1-[(6-cyano-1-naphthyl)sulfonyl]-1,2,4-triazol-3-yl]amino]phthalonitrile; 4-[[5-amino-1-[[6-(cyanomethyl)-1-naphthyl]-sulfonyl]-1,2,4-triazol-3-yl]-amino]benzonitrile; 4-[[5-amino-1-[[7-[2-cyanovinyl]-1-naphthyl]sulfonyl]-1,2,4-triazol-3-yl]amino]-2-chloro-benzonitrile; 4-[[5-amino-1-[[6-[2-cyanovinyl]-1-naphthyl]sulfonyl]-1,2,4-triazol-3-yl]amino]-2-chloro-benzonitrile; and 3-[5-[[5-amino-3-[4-(cyanomethyl)anilino]-1,2,4-triazol-1-yl]sulfonyl]-2-naphthyl]prop-2-enenitrile; or a pharmaceutically acceptable salt thereof.

In some embodiments, the compound is 4-[[5-amino-1-[[6-[2-cyanovinyl]-1-naphthyl]sulfonyl]-1,2,4-triazol-3-yl]amino]-2-chloro-benzonitrile; or a pharmaceutically acceptable salt thereof. In some embodiments, the compound is 4-[[5-amino-1-(6-chloroimidazo[2,1-b]thiazol-5-yl)sulfonyl-1,2,4-triazol-3-yl]amino]-2-chloro-benzonitrile, or a pharmaceutically acceptable salt thereof.

In some embodiments, the presently disclosed subject matter provides a pharmaceutical composition comprising a compound having a structure of Formula (I) and a pharmaceutically acceptable carrier.

In some embodiments, the presently disclosed subject matter provides a method of treating or preventing a viral infection in a subject in need thereof, wherein the method comprises administering to the subject a compound having a structure of Formula (I) or a pharmaceutical composition comprising a compound having a structure of Formula (I) and a pharmaceutically acceptable carrier, optionally wherein the subject is a human. In some embodiments, the viral infection is a human immunodeficiency virus (HIV) infection.

In some embodiments, the presently disclosed subject matter provides a use of a compound having a structure of Formula (I) as a medicament in therapeutic or prophylactic treatment of a viral infection. In some embodiments, the viral infection is a human immunodeficiency virus (HIV) infection. In some embodiments, the medicament is for therapeutic or prophylactic treatment in a human.

In some embodiments, the presently disclosed subject matter provides a use of a compound having a structure of Formula (I) as a medicament for therapeutic treatment of a HIV infection in a human.

Accordingly, it is an object of the presently disclosed subject matter to provide compounds of Formula (I), pharmaceutical compositions thereof, and methods of using the compounds to treat or prevent viral infections, such as HIV infections.

An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIG. 1 is a pair of graphs showing pharmacokinetic data for mice dosed with 250 milligrams per kilogram (mg/kg) of compound 1 via intragastric intubation administration. The graph on the left shows plasma concentration of compound 1 (in nanograms per milliliter (ng/ml)) versus time (in hours (hr)) after dosing and the graph on the right shows brain concentration (in ng/ml) versus time (in hr). Error bars represent standard deviation (SD).

FIGS. 2A-2G are a series of graphs showing human immunodeficiency virus (HIV) inhibition data (percent (%) HIV inhibition) and cytotoxicity data (% cytotoxic) versus concentration (log 10 of compound molar (M) compound concentration) for various compounds in HIV infected cells over an extended dose range. FIG. 2A shows data for (left) efavirenz and (right) rilpivirine inhibition/cytotoxicity in TZM-bl cells infected with wild-type HIV. FIG. 2B shows data for rilpivirine inhibition/cytotoxicity in TZM-bl cells infected with (left) wild-type HIV and (right) TZM-bl cells infected with the A17 variant of HIV. FIG. 2C shows data for compound 2 inhibition/cytotoxicity in TZM-bl cells infected with (left) wild-type HIV and (right) TZM-bl cells infected with the A17 variant of HIV. FIG. 2D shows data for compound 5 inhibition/cytotoxicity in TZM-bl cells infected with (left) wild-type HIV and (right) TZM-bl cells infected with the A17 variant of HIV. FIG. 2E shows data for compound 14 inhibition/cytotoxicity in TZM-bl cells infected with (left) wild-type HIV and (right) TZM-bl cells infected with the A17 variant of HIV. FIG. 2F shows data for compound 20 inhibition/cytotoxicity in TZM-bl cells infected with (left) wild-type HIV and (right) TZM-bl cells infected with the A17 variant of HIV. FIG. 2G shows data for compound 21 inhibition/cytotoxicity in TZM-bl cells infected with (left) wild-type HIV and (right) TZM-bl cells infected with the A17 variant of HIV.

FIG. 3 is a graph showing whole-cell activity for compound 20 at various concentrations (expressed as log 10 of the molar concentration) in cells infected with wild-type (WT) human immunodeficiency virus (HIV) or with a clinically relevant mutant. Percent inhibition of HIV is provided for WT HIV (filled squares), a K103N mutant (circles), a L1001,K103N mutant (x-filled squares), a Y181C mutant (dot-filled squares), and the A17 HIV variant (diamonds). Data is also provided for cytotoxicity (triangles). n≥3.

FIGS. 4A and 4B show the dose dependent toxicity of efavirenz, rilpivirine, and compound 20 as determined by reduction of microtubule-associated protein 2 (MAP-2) staining. FIG. 4A is a graph showing dose dependent (log 10 of compound micromolar (μM) compound concentration) toxicity quantified by relative MAP-2 intensity. MAP-2 area reduction was normalized to untreated cells. Data was fit to a 3-parameter dose response curve due to the variability in the assay. Error bars represent the SEM from 3 replicates. Data for efavirenz is shown in filled circles, data for rilpivirine is shown in filled triangles, and data for compound 20 in x-filled circles. FIG. 4B is a representative microscope image of MAP-2 staining (at 20× magnification).

FIGS. 5A and 5B show calcium accumulation in primary mouse neuron cultures treated or not treated with antiviral compounds. FIG. 5A is a graph showing the increase in the average calcium signaling for all neurons with a segmented x-axis indicating signaling stage (acute stage; depicted as axis with no additional ticks). Neuron treatments include aCSF (media, triangles), vehicle (small squares), compound 20 (stars), rilpivirine (large squares), and efavirenz (EFV, circles). FIG. 5B is a series of graphs showing average acute, delayed, and total calcium spiking in the differently treated neurons described for FIG. 5A. The calcium spiking shows the calcium transients for individual neurons and indicates whether the compounds activate calcium signaling. All compounds were tested at 1 μM. Statistical significance was determined by a Brown-Forsythe and Welch one-way ANOVA test followed by Dunnett's T3 multiple comparison tests as performed in Prism 9.2.0 for Mac OS (GraphPad; San Diego, California, United States of America).

FIGS. 6A and 6B are (FIG. 6A) a schematic drawing of chemical structures for compound 20, rilpivirine, efavirenz (EFV), doravirine, and etravirine; and (FIG. 6B) a graph of reverse transcriptase inhibition (as a function of concentration (log 10 of molar (M) compound concentration)) of the compounds from FIG. 6A. In FIG. 6B, data for EFV is shown in hexagons, data for doravirine is shown in triangles, data for etravirine is shown in diamonds, data for rilpirivine is shown in squares, and data for compound 20 is shown in circles. n 6.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

Unless otherwise indicated, all numbers expressing quantities of time, concentration, dosage and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Heteroaryl” as used herein refers to an aryl group that contains one or more non-carbon atoms (e.g., O, N, S, Se, etc) in the backbone of a ring structure. Nitrogen-containing heteroaryl moieties include, but are not limited to, pyridine, imidazole, benzimidazole, pyrazole, pyrazine, triazine, pyrimidine, and the like.

The term “heterocyclic” refers to a non-aromatic or aromatic mono- or multicyclic ring system of about 3 to about 12 atoms that comprises at least one heteroatom, e.g., N, O, or S. The group can be saturated, partially unsaturated, or unsaturated. Exemplary heterocyclic groups include, but are not limited to, furanyl, pyrrolyl, pyridinyl, pyranyl, piperidinyl, morpholinyl, dioxanyl, pyrrolidinyl, oxanyl, thiolanyl, and thiophenyl. Heterocyclic groups can be unsubstituted or substituted with one or more alkyl group substituents or aryl group substituents.

“Aralkyl” refers to an -alkyl-aryl group, optionally wherein the alkyl and/or aryl moiety is substituted. An exemplary aralkyl group is benzyl, i.e., —CH₂C₆H₅.

“Alkoxyl” or “alkoxyalkyl” refer to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Acylamino” refers to an acyl-NH— group wherein acyl is as previously described.

The term “amino” refers to the group —N(R)₂ wherein each R is independently H, alkyl, substituted alkyl, aryl, substituted aryl, aralkyl, or substituted aralkyl. The terms “aminoalkyl” and “alkylamino” can refer to the group —N(R)₂ wherein each R is H, alkyl or substituted alkyl, and wherein at least one R is alkyl or substituted alkyl. “Dialkylamino” refers to the group —N(R)₂ where each R is alkyl or substituted alkyl. “Arylamino” and “aminoaryl” refer to the group —N(R)₂ wherein each R is H, aryl, or substituted aryl, and wherein at least one R is aryl or substituted aryl, e.g., aniline (i.e., —NHC₆H₅).

The terms “halo”, “halide”, or “halogen” as used herein refer to fluoro, chloro, bromo, and iodo groups.

The term “perhaloalkyl” refers to an alkyl group as described above where each of the hydrogen atoms that are attached to a carbon atom is replaced by halo. A “perfluoroalkyl” group is an exemplary perhaloalkyl group where each hydrogen atom attached to a carbon atom of an alkyl group is replaced by a fluoro group. For example, trifluoromethyl (—CF₃) is an exemplary perfluoroalkyl group.

The term “cyano” refers to the —CN group (i.e., wherein the carbon and nitrogen atoms are bonded to one another via a triple bond).

The term “sulfonyl” refers to the —S(═O)₂— group.

When the term “independently selected” is used, the substituents being referred to (e.g., R groups, such as groups R1 and R2, or groups X and Y), can be identical or different. For example, both R1 and R2 can be substituted alkyls, or R1 can be hydrogen and R2 can be a substituted alkyl, and the like.

A line crossed or terminated by a wavy line, e.g., in the structure:

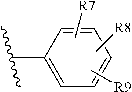

indicates the site where a chemical moiety can bond to another group.

A structure represented generally by a formula:

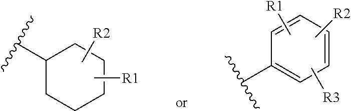

as used herein refers to a ring structure, such as, but not limited to a 3-, 4-, 5-, 6-, 7-, 8-, 9-, or 10-membered cyclic, heterocyclic, aromatic or heteroaromatic moiety comprising substituent groups (e.g., R1, R2, R3, R4, etc.), wherein each substituent group can be substituted on one of the available carbon atoms of the ring structure. Thus each substituent group, if more than one, is substituted on an available carbon of the ring structure rather than on another substituent group. For example, the structure:

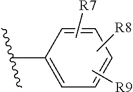

comprises groups including, but not limited to:

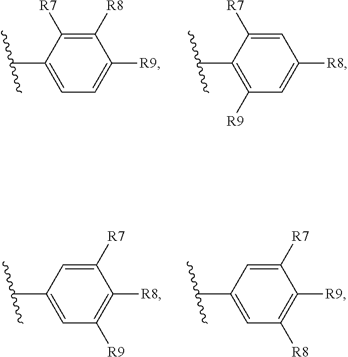

and the like.

The structure:

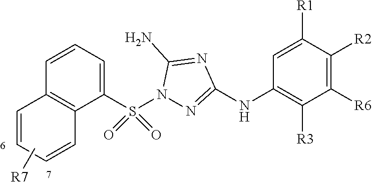

where R7 is substituted on carbon 6 or carbon 7 of the substituted group (i.e., the naphthyl group) refers to the compounds:

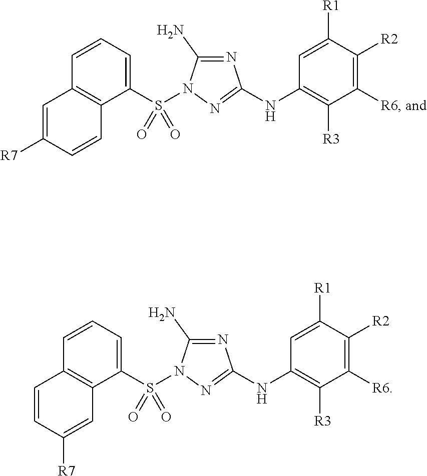

The terms “prophylactic” and “preventing” as used herein refer to a treatment administered to a subject who does not exhibit signs, or exhibits only early signs, of a condition, disease or disorder. Thus, a prophylactic or preventative treatment can be administered for the purpose of decreasing the risk of developing pathology associated with developing the condition, disease or disorder. Accordingly, “prevent” as used herein, means to stop something from happening or taking advance measures against something possible or probable from happening. In the context of medicine, “prevention” generally refers to action taken to decrease the chance of getting a disease or condition. The “prevention” or “prophylaxis” does not need to be absolute, and thus can occur as a matter of degree.

A “therapeutically effective amount” or “effective amount” of a compound is that amount of compound which is sufficient to provide a beneficial effect to the subject to which the compound is administered. In some embodiments, the beneficial effect can be observable or measurable, e.g., a reduction in viral load, mitigation of a symptom, etc.

As used herein, the phrase “therapeutic agent” refers to an agent that is used to, for example, treat, inhibit, mitigate the effects of, prevent, reduce the severity of, slow the progression of, and/or cure, an infection or a disease or disorder.

The terms “treatment” and “treating” as used herein refer to therapeutic treatment measures wherein the object is to slow down (lessen) the targeted pathologic condition, or to pursue or obtain beneficial results, even if the treatment is ultimately unsuccessful. The term “treating” refers to any effect, e.g., lessening, reducing, modulating, ameliorating, reversing or eliminating, that results in the improvement of the condition, disease, disorder, and the like, or ameliorating a symptom thereof.

### II. Antiviral Compounds, Compositions and Methods

To date, six 1st and 2nd generation NNTRI have been approved by the Food and Drug Administration (FDA): nevirapine, efavirenz (EFV), delavirdine, etravirine, doravirine and rilpivirine (38). This class of drug binds in an allosteric pocket of HIV reverse transcriptase inhibiting the progression of viral DNA synthesis (39). Since this class of drugs targets a protein not found in eukaryotes, off target interaction is likely reduced as compared to the nucleoside analog class of inhibitors (NRTIs). First generation NNRTIs have a low genetic barrier to resistance and only require one mutation to confer resistance, while second generation NNRTIs have a higher genetic barrier (40). These compounds are highly potent with low toxicity, yet are still hampered by rapid viral drug resistance, as HIV is highly prone to develop mutational-based drug resistance due to the lack of a proofreading activity of reverse transcriptase (41). Individual clinical isolates have been identified which have resistance to one or more of each of the FDA-approved NNTRIs (38). Thus, there appears to be a need for additional antiviral compounds, particularly those with different chemical core structures and/or which can retain effectiveness against drug-resistant mutants.

### II.A. Antiviral Triazoles

According to one aspect of the presently disclosed subject matter is provided a new class of antiviral triazole compounds. In some embodiments, the antiviral triazole compounds comprise 1,2,4-triazole derivatives, wherein a central 1,2,4-trizazole is substituted by two different aryl or heteroaryl-containing substituents. In some embodiments, the central 1,2,4-triazole is further substituted by an amino group (e.g., a —NH₂ group). In some embodiments, one of the aryl or heteroaryl groups is attached to the central 1,2,4-triazole via a sulfonyl-containing linkage. In some embodiments, one of the aryl or heteroaryl groups is attached to the central 1,2,4-triazole via a divalent group comprising a nitrogen atom. In some embodiments, the divalent group comprising a nitrogen atom is a —NH— group. In some embodiments, one or both of the aryl or heteroaryl groups is further substituted by one or more aryl group substituents, such as a halo group, a cyano group and/or a cyano-substituted group.

In some embodiments, the antiviral triazole compound has a structure of Formula (I):

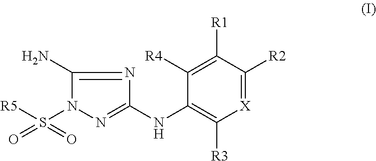

wherein R1, R2, R3, and R4 are independently selected from, H, cyano, cyano-substituted alkyl, and halo; X is CR6 or N, wherein R6 is selected from the group comprising H, cyano, halo, alkyl, alkoxy (e.g., C₁-C₆ alkoxy), cyano-substituted alkyl, dialkylamino, phenyl, substituted phenyl, and aminoacyl; and R5 is a monovalent substituted or unsubstituted aryl or heteroaryl group; or a pharmaceutically acceptable salt thereof.

In some embodiments, R1, R2, R3, and R4 are independently selected from the group comprising H, CN, CH₂CN, CH═CHCN, F, Cl, and lower alkyl. In some embodiments, R6 is selected from the group comprising H, CN, CH₂CN, CH═CHCN, COCH₃, C(CH₃)=CHCN, F, Cl, Br, lower alkyl, MeO, NMe₂, 4-F-Ph, and NMeCOCH═CH₂.

In some embodiments, R5 is a monovalent aryl group selected from:

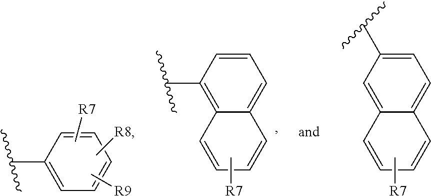

wherein R7, R8, and R9 are independently selected from H, halo, cyano, alkyl, cyano-substituted alkyl, acyl, alkoxy (e.g., C₁-C₆ alkoxy), dialkylamino, aryl, substituted aryl (e.g., halo-substituted aryl), and aminoacyl; or R5 is a monovalent heteroaryl group selected from the group consisting of:

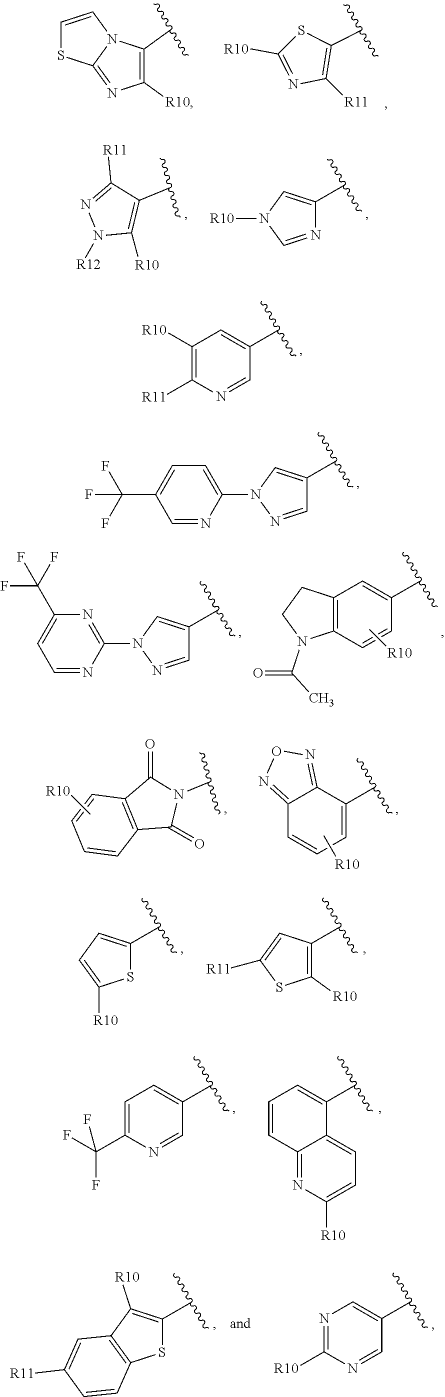

wherein R10, R11, and R12 are independently selected from the group comprising H, halo, cyano, alkyl, alkoxy (e.g., C₁-C₆ alkoxy), perhaloalkyl (e.g., perfluoroalkyl), and aminoacyl.

In some embodiments, R7, R8, and R9 are independently selected from the group comprising H, CN, CH₂CN, CH═CHCN, COCH₃, C(CH₃)=CHCN, F, Cl, Br, lower alkyl, MeO, NMe₂, 4-F-Ph, and NMeCOCH═CH₂. In some embodiments, R10, R11, and R12 are independently selected from the group comprising H, F, Cl, Br, CN, lower alkyl, MeO, CF₃, and NHCOCH₃.

In some embodiments, R5 is:

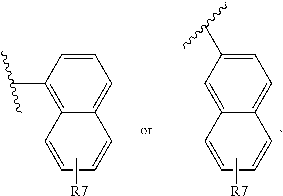

where R7 is selected from the group comprising H, CN, cyano-substituted alkyl (e.g., CH₂CN, CH═CHCN, or C(CH₃)=CHCN), acyl (e.g., COCH₃), halo (e.g., F, Cl, or Br), lower alkyl, alkoxy (e.g., MeO), dialkylamino (e.g., NMe₂), halo-substituted phenyl (e.g., 4-fluorophenyl (4-F-Ph)), and aminoacyl (e.g., NMeCOCH═CH₂).

In some embodiments, at least R2 is other than H. In some embodiments, R2 is selected from CN, CH₂CN, CH═CHCN, F, Cl, and lower alkyl.

In some embodiments, R5 is:

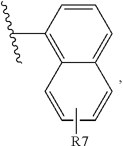

X is CR6, and R4 is H, and the compound of Formula (I) has a structure of the formula:

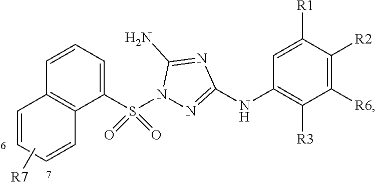

wherein R1, R2, and R3 are independently selected from the group comprising H, cyano, alkyl, cyano-substituted alkyl, and halo (e.g., H, CN, CH₂CN, CH═CHCN, F, Cl, or lower alkyl); R6 is selected from the group comprising H, cyano, alkyl, cyano-substituted alkyl, halo, alkoxy, dialkylamino, halo-substituted phenyl, and aminoacyl (e.g., H, CN, CH₂CN, CH═CHCN, COCH₃, C(CH₃)=CHCN, F, Cl, Br, lower alkyl, MeO, NMe₂, 4-F-Ph, or NMeCOCH═CH₂) and R7 is selected from the group comprising H, cyano, alkyl, cyano-substituted alkyl, halo, alkoxy, dialkylamino, halo-substituted phenyl, and aminoacyl (e.g., H, CN, CH₂CN, CH═CHCN, COCH₃, C(CH₃)=CHCN, F, Cl, Br, lower alkyl, MeO, NMe₂, 4-F-Ph, or NMeCOCH═CH₂). In some embodiments, R7 is substituted on the naphthyl group ring that is not directly attached to the sulfonyl group. In some embodiments, R7 is attached to carbon 6 or carbon 7.

In some embodiments, R1 and R3 are each H. In some embodiments, R2 is selected from CN, CH₂CN, and CH═CHCN and R6 is selected from H, CN, and Cl. In some embodiments, R2 is CN and R6 is Cl. In some embodiments, R2 is CN and R6 is CN. In some embodiments, R2 is CH₂CN and R6 is H. In some embodiments, R7 is CH═CHCN.

In some embodiments, R5 is

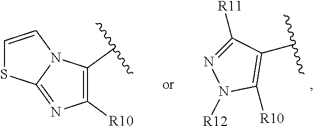

wherein R10, R11, and R12 are independently selected from the group comprising H, halo, cyano, alkyl, alkoxy (e.g., C₁-C₆ alkoxy), perhaloalkyl, and aminoacyl. In some embodiments, R10, R11, and R12 are independently selected from H, F, Cl, Br, CN, lower alkyl, MeO, CF₃, and NHCOCH₃. In some embodiments, R11 and R12 are each lower alkyl (e.g., methyl). In some embodiments, R10 is selected from H and Cl.

In some embodiments, X is CR6 and the compound has a structure of the formula:

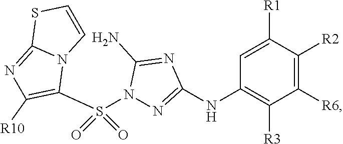

wherein R1, R2, and R3 are independently selected from the group comprising H, cyano, halo, alkyl, and cyano-substituted alkyl (e.g., H, CN, CH₂CN, CH═CHCN, F, Cl, or lower alkyl); R6 is selected from the group comprising H, halo, cyano, alkyl, cyano-substituted alkyl, alkoxy, acyl, dialkylamino, halo-substituted phenyl, and aminoacyl (e.g., H, CN, CH₂CN, CH═CHCN, COCH₃, C(CH₃)=CHCN, F, Cl, Br, lower alkyl, MeO, NMe₂, 4-F-Ph, or NMeCOCH═CH₂); and R10 is selected from the group comprising H, halo, cyano, alkyl, alkoxy, perhaloalkyl, and aminoacyl (e.g., H, F, Cl, Br, CN, lower alkyl, MeO, CF₃, or NHCOCH₃). In some embodiments, R10 is selected from H and CI. In some embodiments, R1 and R3 are each H; R2 is selected from CN, CH₂CN, and CH═CHCN; and R6 is selected from H, CN, and Cl. In some embodiments, R10 is Cl. In some embodiments, R2 is CN and R6 is H.

In some embodiments, the compound of Formula (I) has a structure of one of the compounds of Examples 1-53, below, or a pharmaceutically acceptable salt thereof. In some embodiments, the compound of Formula (I) is selected from the group comprising: 5-[[5-amino-3-(4-cyanoanilino)-1,2,4-triazol-1-yl]sulfonyl]naphthalene-2-carbonitrile (compound 2); 5-[[5-amino-3-(3-chloro-4-cyano-anilino)-1,2,4-triazol-1-yl]sulfonyl]naphthalene-2-carbonitrile (compound 5); 4-[[5-amino-1-(6-chloroimidazo[2,1-b]thiazol-5-yl)sulfonyl-1,2,4-triazol-3-yl]amino]-2-chloro-benzonitrile (compound 11); 4-[[5-amino-1-[(6-cyano-1-naphthyl)sulfonyl]-1,2,4-triazol-3-yl]amino]phthalonitrile (compound 14); 4-[[5-amino-1-[[6-(cyanomethyl)-1-naphthyl]-sulfonyl]-1,2,4-triazol-3-yl]amino]benzonitrile (compound 15); 4-[[5-amino-1-[[7-[2-cyanovinyl]-1-naphthyl]sulfonyl]-1,2,4-triazol-3-yl]amino]-2-chloro-benzonitrile (compound 19); 4-[[5-amino-1-[[6-[2-cyanovinyl]-1-naphthyl]sulfonyl]-1,2,4-triazol-3-yl]amino]-2-chloro-benzonitrile (compound 20); and 3-[5-[[5-amino-3-[4-(cyanomethyl)anilino]-1,2,4-triazol-1-yl]sulfonyl]-2-naphthyl]prop-2-enenitrile (compound 21); or a pharmaceutically acceptable salt thereof.

In some embodiments, the compound of Formula (I) is 4-[[5-amino-1-[[6-[2-cyanovinyl]-1-naphthyl]sulfonyl]-1,2,4-triazol-3-yl]amino]-2-chloro-benzo-nitrile; or a pharmaceutically acceptable salt thereof. In some embodiments, the compound of Formula (I) is 4-[[5-amino-1-(6-chloroimidazo[2,1-b]thiazol-5-yl)sulfonyl-1,2,4-triazol-3-yl]amino]-2-chloro-benzonitrile, or a pharmaceutically acceptable salt thereof.

### II.B. Synthesis

In some embodiments, the presently disclosed subject matter provides a method of preparing a compound of Formula (I). For example, in some embodiments, the presently disclosed subject matter relates to an effective method for synthesizing derivatives of N-aryl-1-(arylsulfonyl)-1H-1,2,4-triazoles with the ability of introducing targeted replacements into the molecule structure. Key intermediates in the preparation of the compounds are methyl N′-cyano-N-arylimidothiocarbamates, which can be prepared by different synthetic routes. Scheme 1, below, shows the synthesis of an exemplary methyl N′-cyano-N-arylimidothiocarbamate intermediate, i.e., a methyl N′-cyano-N-phenylimidothiocarbamate. A first approach to the intermediate is based on the reaction of an aniline with dimethyl cyanothioimidocarbonate and elimination methylmercaptane (step (a) of Scheme 1). This approach is suitable for aniline starting materials comprising electron donating or neutral substituents.

To synthesize intermediates having electron-withdrawing groups substituted in the aniline fragment, two alternative approaches were developed. Both alternative approaches are based on the initial reaction of an aniline, which can contain electron-donating or electron-withdrawing substituents. In the first alternative approach, the aniline was reacted with dimethylaminothiocarbamoyl chloride in an anhydrous medium (step (b) of Scheme 1). In this case, the corresponding aryl isothiocyanate is obtained in quantitative yield and subsequently reacted with cyanamide in the presence of sodium ethoxide and then with methyl iodide (step (d) in Scheme 1) resulting in formation of the intermediate N′-cyano-N-phenylimidothiocarbamate.

The same intermediates can be synthesized by a second alternative approach by the reaction of an aniline with thiophosgene under alkali conditions, for example in the presence an base/alkali compound (e.g., triethylamine, diisopropylethylamine, sodium methoxide or calcium carbonate, etc.) in an anhydrous solvent, with formation of the corresponding isothiocyanate (step (c) of Scheme 1) and followed by transformation to the N′-cyano-N-phenylimidothiocarbamate via reaction with cyanamide (step (d) of Scheme 1). All three approaches work well with high yield and purity of target compounds.

The reaction of the intermediate methyl N′-cyano-N-phenylimidothiocarbamate with hydrazine hydrate (step (e) of Scheme 1) leads to closure of the 1,2,4-triazole ring. The last step (step (f) of Scheme 1) of the sequence is with an arylsulfonyl chloride. While this last step usually leads to a mixture of two isomers, conditions can be selected for the reaction and/or crystallization of the mixture, at which the concentration of the desired isomer (e.g., the N3-phenyl-1-(phenylsulfonyl)-1H-1,2,4-triazole-3,5-diamine of Scheme 1) significantly prevails. Alternatively, the isomers can be separated by chromatography.

All compounds were obtained at a purity of not less than 98% in amounts sufficient to carry out all planned biological experiments. The confirmation of the structure and investigation of the physicochemical properties of all synthesized compounds was performed by various methods known in the art and as described hereinbelow in the Examples.

### II.C. Pharmaceutical Compositions

Surprisingly the compounds of the presently disclosed subject matter exhibit strong antiviral activity, particularly against HIV. Thus, the compounds of the presently disclosed subject matter are useful for the treatment of viral infections in humans and in animals.

In some embodiments, the presently disclosed subject matter provides a pharmaceutical composition comprising a compound having a structure of Formula (I). For example, the pharmaceutical composition can include one or more compounds of Formula (I) and a pharmaceutically acceptable carrier. In some embodiments, the compounds of the presently disclosed subject matter are formulated for use by preparing a dilute solution or suspension in a pharmaceutically acceptable aqueous, organic, or aqueous-organic medium. In some embodiments, the compounds are formulated for topical or parenteral administration by intravenous, subcutaneous or intramuscular injection, or for intranasal application or for intracerebroventricular or intrathecal administration; or are prepared in tablet, capsule or aqueous suspension form with conventional excipients for oral administration or as a suppository.

Accordingly, the compounds of the presently disclosed subject matter can be formulated, for example, in a wide variety of oral administration dosage forms and carriers. Oral administration can be in the form of tablets, coated tablets, dragée, hard and soft gelatin capsules, solutions, emulsions, syrups or suspensions. Compounds of the presently disclosed subject matter are also efficacious when administered by other routes of administration, including, for example, continuous (intravenous drip), topical parenteral, intramuscular, intravenous, subcutaneous, transdermal (e.g., alone or in combination with a penetration enhancement agent), buccal, nasal, inhalation, and suppository routes, among other routes of administration. In some embodiments, the compounds are administered orally, e.g., using a daily or weekly dosing regimen.

Thus, in some embodiments, the compounds of the presently disclosed subject matter are provided for use in the treatment or prevention of viral infections, particularly retroviral infections, in humans and in animals (e.g., non-human mammals). In some embodiments, the compounds of the presently disclosed subject matter are provided for use in the treatment or prevention of a disease or condition caused by (or triggered by) a viral infection, such as for use in the treatment or prevention of a disease or condition caused by a retrovirus infection, in humans and in animals.

In some embodiments, the presently disclosed subject matter relates the use of a compound of Formula (I) in a method for the treatment or prophylaxis of viral infections in animals (e.g., humans or other mammals) or for the treatment or prophylaxis of a disease or condition caused or triggered by a viral infection. In some embodiments, the viral infection is a retroviral infection. In some embodiments, the retroviral infection is a HIV infection. In some embodiments, the HIV infection is an HIV-1 infection. In some embodiments, the HIV infection is an HIV-2 infection. In some embodiments, the disease caused by the HIV infection is AIDS. Thus, in some embodiments, the presently disclosed compounds are provided for use in treating or preventing HIV and/or AIDS. In some embodiments, the retroviral infection is a human T-lymphotropic virus (HTLV) infection (e.g., a HTLV type 1, type 11, type III or type IV infection). In some embodiments, the disease caused by the HTLV infection is a cancer, e.g., adult T-cell leukemia/lymphoma. In some embodiments, the retrovirus infection is a simian T-lymphotropic virus (STLV) infection or a simian immunodeficiency virus (SIV). In some embodiments, the retrovirus infection is a bovine leukemia virus infection (BLV), a feline leukemia virus infection (FLV) or a feline immunodeficiency virus (FIV) infection.

In some embodiments, the compounds of Formula (I) can be used in dosages from 0.001-1000 mg/kg body weight. In some embodiments, the compounds of Formula (I) can be used in dosages from about 0.01-about 1000 mg/kg body weight. In some embodiments, the compound of Formula (I) is used in combination with one or more additional therapeutic agents, e.g., one or more additional antiviral or antimicrobial therapeutic agents (i.e., an additional antiviral therapeutic agent that is not a compound of Formula (I)) and/or one or more additional therapeutic agents used to treat a symptom of a viral infection or a disease caused or triggered by a viral infection or to enhance the immune response of a subject.

As noted above, in some embodiments, the compound of Formula (I) can be provided as a pharmaceutically acceptable salt. Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N, N′-dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like.

Examples of metal salts include lithium, sodium, potassium, and magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

In some embodiments, the presently disclosed compounds can further be provided as a solvate.

The compound of Formula (I) can be used on a sample either in vitro (for example, on isolated cells or tissues) or in vivo in a subject (i.e. living organism, such as a patient). In some embodiments, the subject or patient is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in warm-blooded vertebrates. Thus, the presently disclosed subject matter concerns mammals and birds. More particularly provided are methods and compositions for mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, also provided is the treatment of livestock including, but not limited to domesticated swine (pigs and hogs), ruminants, horses, poultry, and the like.

In some embodiments, the compound of Formula (I) can include more than one of the compounds described herein. In some embodiments, the compound can be administered along with one or more additional therapeutic agents known in the art for treating a disease or disorder associated with a viral infection. For example, the compounds can be co-administered with an antiviral compound that is not a compound of Formula (I), an antimicrobial compound, or a therapeutic agent useful in treating a symptom of a viral infection (e.g., pain, fever, inflammation, etc.). The compound of Formula (I) and the one or more other therapeutic agents can be provided in a single formulation or co-administered in separate formulations at about the same time or at different times (e.g., different times within the same day, week, or month).

In some embodiments, the compound of Formula (I) (which can also be referred to as the “active ingredient”) can be administered in a pharmaceutically acceptable composition where the compound can be admixed with one or more pharmaceutically acceptable carriers. The term “pharmaceutically acceptable carrier” means a non-toxic material that does not interfere with the effectiveness of the biological activity of the active ingredients. In some embodiments, the pharmaceutically acceptable composition can also contain salts, buffering agents, preservatives, compatible carriers, and optionally other therapeutic agents.

In some embodiments, the methods for administration of a compound of Formula (I) or pharmaceutically acceptable composition thereof to a subject include, but are not limited to intravenous injection, oral administration, buccal, topical, subcutaneous administration, intraperitoneal injection, pulmonary, intanasal, intracranial injection, and rectal administration. The particular mode of administering a composition matter depends on various factors, including the distribution and abundance of cells to be treated and mechanisms for metabolism or removal of the composition from its site of administration.

An effective dose of a composition of the presently disclosed subject matter is administered to a subject. In some embodiments, an “effective amount” is an amount of the composition sufficient to produce detectable treatment. Actual dosage levels of constituents of the compositions of the presently disclosed subject matter can be varied so as to administer an amount of the composition that is effective to achieve the desired effect for a particular subject and/or target. The selected dosage level can depend upon the activity of the composition and the route of administration. In some embodiments, the compounds of Formula (I) can be used in dosages from 0.001-1000 mg/kg body weight.

After review of the disclosure herein of the presently disclosed subject matter, one of ordinary skill in the art can tailor the dosages to an individual subject, taking into account the particular formulation, method of administration to be used with the composition, and nature of the target to be treated. Such adjustments or variations, as well as evaluation of when and how to make such adjustments or variations, are well known to those of ordinary skill in the art.

The therapeutically effective amount can be determined by testing the compounds in an in vitro or in vivo model and then extrapolating therefrom for dosages in subjects of interest, e.g., humans. The therapeutically effective amount should be enough to exert a therapeutically useful effect in the absence of undesirable side effects in the subject to be treated with the composition.

Pharmaceutically acceptable carriers are well known to those skilled in the art and include, but are not limited to, from about 0.01 to about 0.1M and preferably 0.05M phosphate buffer or 0.8% saline. Such pharmaceutically acceptable carriers can be aqueous or non-aqueous solutions, suspensions and emulsions. Examples of non-aqueous solvents suitable for use in the presently disclosed subject matter include, but are not limited to, propylene glycol, polyethylene glycol, vegetable oils such as olive oil, and injectable organic esters such as ethyl oleate. Aqueous carriers suitable for use in the presently disclosed subject matter include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the presently disclosed subject matter can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the presently disclosed subject matter include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the presently disclosed subject matter include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Parenteral carriers suitable for use in the presently disclosed subject matter include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the presently disclosed subject matter can be mixed as needed with disintegrants, diluents, granulating agents, lubricants, binders and the like using conventional techniques known in the art. The carriers can also be sterilized using methods that do not deleteriously react with the compounds, as is generally known in the art. The compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Further, formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compounds can further be formulated for topical administration. Suitable topical formulations include one or more compounds in the form of a liquid, lotion, cream or gel. Topical administration can be accomplished by application directly on the treatment area. For example, such application can be accomplished by rubbing the formulation (such as a lotion or gel) onto the skin of the treatment area, or by spray application of a liquid formulation onto the treatment area.

In some formulations, bioimplant materials can be coated with the compounds so as to improve interaction between cells and the implant.

Formulations of the compounds can contain minor amounts of wetting or emulsifying agents, or pH buffering agents. The formulations comprising the compound can be a liquid solution, suspension, emulsion, tablet, pill, capsule, sustained release formulation, or powder. The compounds can be formulated as a suppository, with traditional binders and carriers such as triglycerides.

Oral formulations can include standard carriers such as pharmaceutical grades of mannitol, lactose, starch, magnesium stearate, polyvinyl pyrollidone, sodium saccharine, cellulose, magnesium carbonate, etc.

In some embodiments, the pharmaceutical composition comprising the compound of Formula (I) of the presently disclosed subject matter can include an agent which controls release of the compound, thereby providing a timed or sustained release compound.

### II.D. Methods of Treating or Preventing Viral Infections and Related Diseases

In some embodiments, the presently disclosed subject matter provides a method of treating or preventing a viral infection in a subject in need of thereof, wherein the method comprises administering to the subject a compound of Formula (I) or a pharmaceutical composition thereof. In some embodiments, the subject is a mammal. In some embodiments, the subject is a human. In some embodiments, the viral infection is a retroviral infection. In some embodiments, the viral infection is a HIV infection (e.g., a HIV-1 or HIV-2 infection). In some embodiments, the viral infection is a HTLV infection. In some embodiments, the viral infection is a STLV, SIV, BLV, FLV or FIV infection.

In some embodiments, the presently disclosed subject matter provides the use of a compound having a structure of Formula (I) as described herein above as a medicament (or in preparing a medicament) for therapeutic or prophylactic treatment of a viral infection. In some embodiments, the compound has a structure:

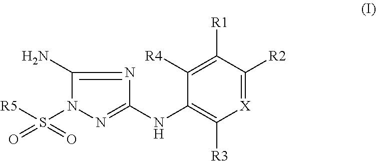

wherein R1, R2, R3, and R4 are independently selected from the group comprising H, CN, CH₂CN, CH═CHCN, F, Cl, and lower alkyl; X is CR6 or N, wherein R6 is selected from the group comprising H, CN, CH₂CN, CH═CHCN, COCH₃, C(CH₃)=CHCN, F, Cl, Br, lower alkyl, MeO, NMe₂, 4-F-Ph, and NMeCOCH═CH₂; and R5 is a monovalent aryl group selected from the group comprising:

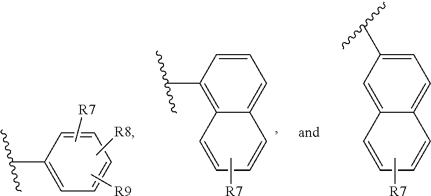

wherein R7, R8, and R9 are independently selected from the group comprising H, CN, CH₂CN, CH═CHCN, COCH₃, C(CH₃)=CHCN, F, Cl, Br, lower alkyl, MeO, NMe₂, 4-F-Ph, and NMeCOCH═CH₂; or wherein R5 is a monovalent heteroaryl group selected from the group comprising:

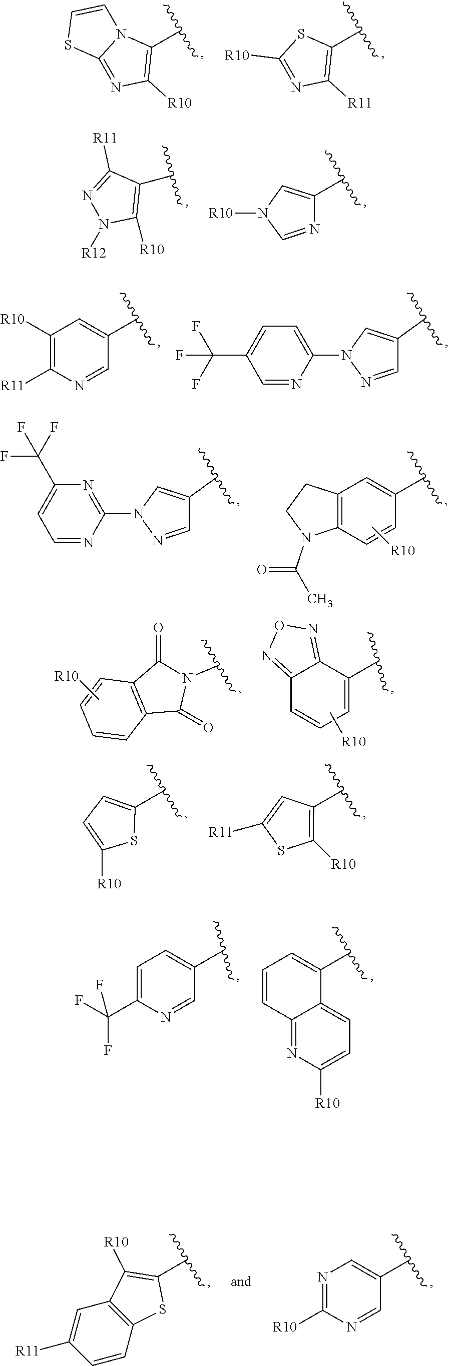

wherein R10, R11, and R12 are independently selected from the group comprising H, F, Cl, Br, CN, lower alkyl, MeO, CF₃, and NHCOCH₃; or a pharmaceutically acceptable salt thereof.

In some embodiments, the viral infection is a retroviral infection. In some embodiments, the viral infection is a HIV or HTLV infection. In some embodiments, the viral infection is a HIV infection. In some embodiments, the HIV infection is an HIV-1 infection. In some embodiments, the HIV infection is an HIV-2 infection. In some embodiments, the viral infection is an HTLV infection. In some embodiments, the viral infection is a STLV, SIV, BLV, FLV, or FIV infection.

In some embodiments, the medicament is for therapeutic treatment. In some embodiments, the medicament is for prophylactic treatment. In some embodiments, the medicament is for therapeutic or prophylactic treatment in a mammal. In some embodiments, the medicament is for therapeutic or prophylactic treatment of a human.

In some embodiments, the presently disclosed subject matter provides the use of compound having a structure of Formula (I):

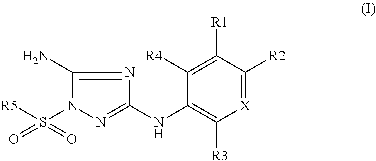

wherein R1, R2, R3, and R4 are independently selected from the group comprising H, CN, CH₂CN, CH═CHCN, F, Cl, and lower alkyl; X is CR6 or N, wherein R6 is selected from the group comprising H, CN, CH₂CN, CH═CHCN, COCH₃, C(CH₃)=CHCN, F, Cl, Br, lower alkyl, MeO, NMe₂, 4-F-Ph, and NMeCOCH═CH₂; and R5 is a monovalent aryl group selected from the group comprising:

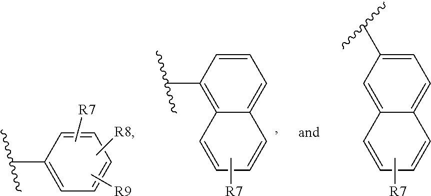

wherein R7, R8, and R9 are independently selected from the group consisting of H, CN, CH₂CN, CH═CHCN, COCH₃, C(CH₃)=CHCN, F, Cl, Br, lower alkyl, MeO, NMe₂, 4-F-Ph, and NMeCOCH═CH₂; or wherein R5 is a monovalent heteroaryl group selected from the group comprising:

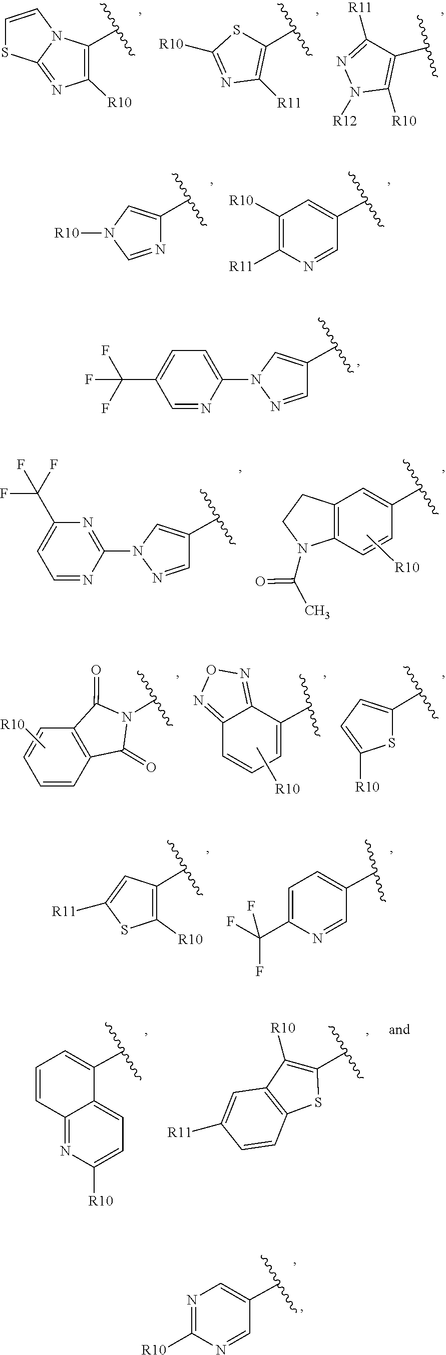

wherein R10, R11, and R12 are independently selected from the group comprising H, F, Cl, Br, CN, lower alkyl, MeO, CF₃, and NHCOCH₃; or a pharmaceutically acceptable salt thereof; as a medicament for therapeutic treatment of a HIV infection in a human.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### General Synthetic Methodology

### Starting Materials

All reagents and solvents were purchased from commercial suppliers and used without further purification. ¹H and ¹³C Spectra were measured on Bruker AC-500 (500 MHz, ¹H) or Bruker AC-200 (75 MHz, ¹³C) (Bruker Corperation, Billerica, Massachusetts, United States of America). Chemical shifts were measured in DMSO-d₆ or CDCl₃, using tetramethylsilane as an internal standard, and reported as units (ppm) values. The following abbreviations are used to indicate the multiplicity: s, singlet; d, doublet; t, triplet; m, multiplet; dd, doublet of doublets; brs, broad singlet; brm, broad multiplet. Mass spectra were recorded on Finnigan MAT INCO 50 mass spectrometer (EI, 70 eV; Thermo Finnigan LLC, San Jose, California, United States of America) with direct injection.

The purity of the final compounds were analyzed on Agilent 1290 Infinity II HPLC system coupled to Agilent 6460 triple-quadrupole mass spectrometer (Agilent Technologies, Santa Clara, California, United States of America) equipped with an electrospray ionization source. The chromatographic separation was carried out on Agilent Eclipse Plus C18 RRHD column (2.1×50 mm, 1.8 μm; Agilent Technologies, Santa Clara, California, United States of America) at 40° C., sample injection volume was 0.2 μL. The mobile phase comprising 0.1% formic acid/water (A), and 0.1% formic acid and 85% acetonitrile/water (B) was programmed with gradient elution (0.0-3.0 min, 60% B; 3.0-4.0 min, 60% to 97% B; 4.0-6.0 min, 97% B; 6.0-6.1 min, 97% to 60% B) at a flow rate of 0.4 mL/min. The mass spectrometric detection was operated in positive ion mode. Optimal parameters were: capillary voltages of 3500 V, a nebulizer pressure of 35 psi, a gas temperature of 350° C., a gas flow rate of 12/min. All final compounds are >95% pure.

Melting points were determined on an Electrothermal 9001 (10° C. per min) melting point apparatus and are uncorrected. Merck KGaA silica gel 60 F254 plates (Merck KGaA, Darmstadt, Germany) were used for analytical thin-layer chromatography. Column chromatography was performed on Merck silica gel 60 (70-230 mesh; Merck KGaA, Darmstadt, Germany). Yields refer to purified products and are not optimized.

The starting materials and the intermediates of the synthetic reaction schemes also can be isolated and purified if desired using conventional techniques, including but not limited to, filtration, distillation, crystallization, chromatography, and the like. Such materials can be characterized using conventional means, including physical constants and spectral data.

Exemplary of representative compounds encompassed by the presently disclosed subject matter and within the scope of the presently disclosed subject matter are provided in the following examples. These examples and preparations which follow are provided to enable those skilled in the art to more clearly understand and to practice the presently disclosed subject matter. They should not be considered as limiting the scope of the presently disclosed subject matter, but merely as being illustrative and representative thereof.

All compounds of Examples 1-53 were synthesized according to the general synthetic procedure for the synthesis of 1,2,4-triazole derivatives outlined in Scheme 1, below.

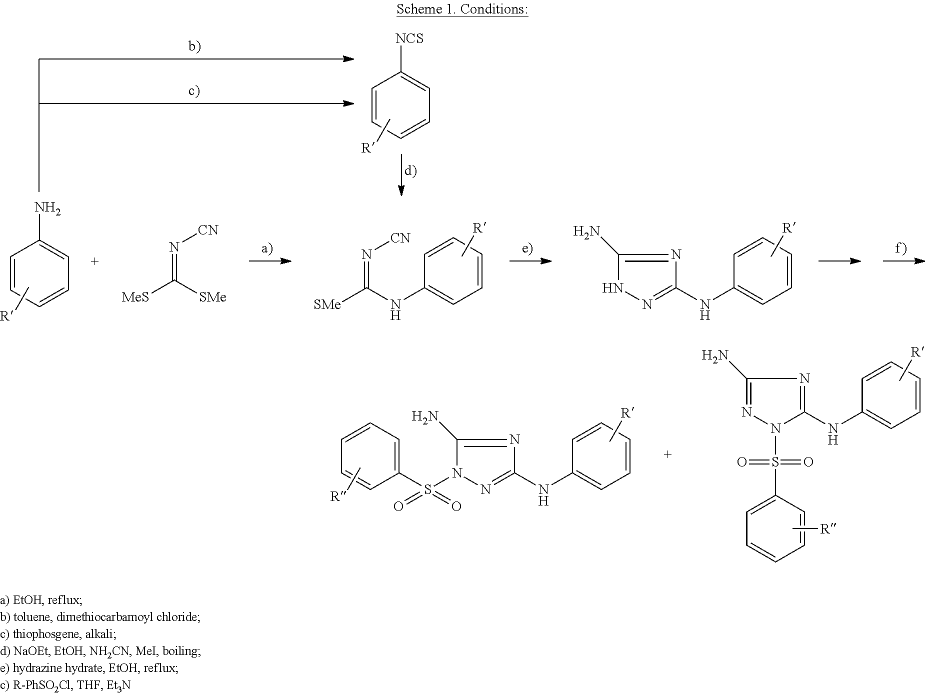

General conditions for steps a-f in Scheme 1 are as follows:

Step a). A mixture of corresponding aniline (1.0-1.2 mmol) and dimethyl cyanoditioiminocarbonate (1.0 mmol) in small volume of n-butanol was refluxed for 3-4 hours. The reaction mixture was cooled and the formed precipitate was filtered off and washed with hexane. The desired methyl N′-cyano-N—R-phenylimidothiocarbamate was recrystallized from ethanol.

Step b). A solution of aniline derivative (1.0 mmol) in dry toluene or benzene was treated with solid dimethylthiocarbamoylchloride (1.0-1.1 mmol) and was refluxed for 2-3 hours. The reaction mixture was cooled, dissolved by hexane and the formed solid was filtered off. The mother solution was evaporated in vacuum and the desired isothiocyanate derivative was used without additional purification.

Step c). A mixture of substituted aniline (1.0 mmol) and suitable alkali (such as triethylamine, diisopropylethylamine, calcium carbonate, potassium carbonate, etc. (1.8-2.2 mmol)) in toluene, benzene or CH₂Cl₂ was treated with thiophosgene (1.0 mmol) and stored for 2 hours at room temperature or refluxed for 4-6 hours. The reaction mixture was diluted in water, and the organic phase was separated, concentrated in vacuum, and chromatographed (e.g., using benzene as the eluant) to give the desired isothiocyanate derivative as a light yellow or white solid.

Step d). A mixture of sodium ethoxide (2.0-2.2 mmol) in 20 ml of ethanol and cyanamide (2.0 mmol) was stirred at room temperature for 30-40 minutes. The isothiocyanate (2.0-2.2 mmol) from step b) or c) was added to the reaction mixture and stirred for 1.5 hours. Iodomethane (4.0-4.5 mmol) was added to reaction mixture and the mixture was refluxed for 1-2 hours and stored overnight at room temperature. The resulting residue was filtered off and dried to give the methyl N′-cyano-N—R-phenylimidothiocarbamate.

Step e). A water solution of hydrazine (3.0-5.0 mmol) was added to a solution of methyl N-4-bromo-3,5-dichlorophenyl-N′-cyanocarbamimidothioate (1.0-1.5) in ethanol and heated at 70° C. for 3-4 hours. The reaction mixture was cooled to room temperature and dissolved by ice water. The precipitate was collected and recrystallized from ethanol to give the N⁵-phenyl-1H-1,2,4-triazole-3,5-diamine as an off-white solid.

Step f) High quality sulfonylchloride derivative (1.0-1.2 mmol) was added to a suspension of the N5-phenyl-1H-1,2,4-triazole-3,5-diamine (1.0 mmol) in a small volume of pyridine. The reaction mixture was stored overnight at room conditions, diluted in water, cooled at 4° C. for 6-24 hours, and the precipitate was filtered off. The desired N3-(4-phenyl)-1-(2-R6-sulfonyl)-1,2,4-triazole-3,5-diamine isomer was separated from byproduct by recrystallization (using EtOH or EtOH/DMF) or by chromatography (e.g., with chloroform:methanol 10/1 as the eluant).

### Example 1

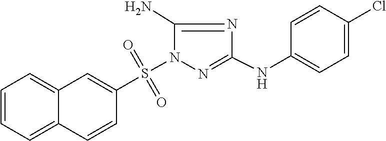 [SMILES: Nc1nc(Nc2ccc(Cl)cc2)nn1S(=O)(=O)c1ccc2ccccc2c1]

### N3-(4-chlorophenyl)-1-(2-naphthylsulfonyl)-1,2,4-triazole-3,5-diamine (Compound 1) Compound 1

Yield 55%. Mass (EI), m/z (Irelat.(%)): 399.8550 [M]+ (59). C₁₈H₁₄ClN₅O₂S. ¹H NMR (500 MHz, DMSO-d₆) δ 9.28 (s, 1H, NH), 8.72 (s, 1H, HC(1′)), 8.23 (d, J=7.8 Hz, 1H, HC(8′)), 8.18 (d, J=8.8 Hz, 1H, C(4′)), 8.05 (d, J=8.0 Hz, 1H, HC(5′)), 7.90 (dd, J=8.7, 1.8 Hz, 1H, HC(3′)), 7.82-7.67 (m, 2H, HC(6′,7′)), 7.45 (d, J=8.9 Hz, 2H, HC(3″,5″)), 7.40 (s, 2H, NH₂), 7.26 (d, J=8.9 Hz, 2H, HC(2″,6″)). ¹³C NMR (75 MHz, DMSO-d₆) δ 159.12 (C-3), 156.89 (C-5), 139.64 (C-1″), 134.89 (C-4a′), 133.10 (C-2′), 131.65 (C-8a′), 129.76 (C-3′), 129.86 (C-4′), 128.97 (C-5′), 128.35 (C-3″, 5′), 128.25 (C-1′), 127.97 (C-6′), 123.69 (C-4″), 122.12 (C-7′), 121.65 (C-8′), 118.12 (C-2″, 6″).

### Example 2

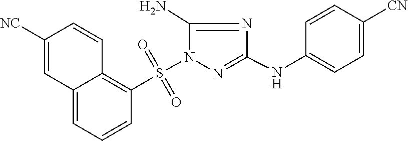 [SMILES: N#Cc1ccc(Nc2nc(N)n(S(=O)(=O)c3cccc4cc(C#N)ccc34)n2)cc1]

### 5-[[5-amino-3-(4-cyanoanilino)-1,2,4-triazol-1-yl]sulfonyl]naphthalene-2-carbonitrile (Compound 2)

Yield 47%. Mass (EI), m/z (Irelat.(%)): 415.4292 [M]+ (73). C₂₀H₁₃N₇O₂S. ¹H NMR (500 MHz, DMSO) δ 9.79 (s, 1H, NH), 9.12 (d, J=9.1 Hz, 1H, HC(4′)), 8.76 (d, J=1.7 Hz, 1H, HC(1′)), 8.64 (d, J=7.2 Hz, 1H, HC(6′)), 8.50 (d, J=8.0 Hz, 1H, HC(8′)), 8.15 (dd, J=9.1, 1.9 Hz, 1H, HC(3′)), 7.92 (t, J=7.9 Hz, 1H, HC(7′)), 7.67 (d, J=8.8 Hz, 2H, HC(3″), HC(5″)), 7.56 (s, 2H, NH₂), 7.51 (d, J=8.9 Hz, 2H, HC(2″), HC(6″)). ¹³C NMR (50 MHz, DMSO) δ 158.51 (C-3), 156.33 (C-5), 144.53 (C-1″), 136.98 (C-5′), 135.32 (C-1′), 133.56 (C-4a′), 133.01 (C-3′, C-5″), 132.86 (C-8a′), 132.28 (C-3′), 128.92 (C-8′), 126.77 (C-6′), 126.55 (C-7′), 126.31 (C-4′), 119.49 (CN—C2′), 118.12 (CN—C4′), 116.61 (C-2″, C-6″), 110.18 (C-1′), 101.60 (C-4″).

### Example 3

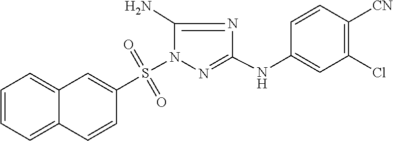 [SMILES: N#Cc1ccc(Nc2nc(N)n(S(=O)(=O)c3ccc4ccccc4c3)n2)cc1Cl]

### 4-[[5-amino-1-(2-naphthylsulfonyl)-1,2,4-triazol-3-yl]amino]-2-chloro-benzonitrile (Compound 3)

Yield 45%. Mass (EI), m/z (Irelat.(%)): 424.8645 [M]+ (33). C₁₉H₁₃ClN₆O₂S. ¹H NMR (500 MHz, DMSO) δ 10.09 (s, 1H, NH), 8.75 (s, 1H, HC(1′)), 8.35-7.32 (m, 10H). ¹³C NMR (126 MHz, DMSO) δ 158.58 (C-3), 157.26 (C-5), 145.82 (C-4″), 135.96 (C-2″), 135.10 (C-4a′), 134.94 (C-6″), 132.88 (C-2′), 131.37 (C-8a′), 130.03 (C-3′), 129.96 (C-4′), 129.57 (C-5′), 128.18 (C-1′), 127.97 (C-6′), 121.65 (C-8′), 116.64 (CN), 116.35 (C-3″), 115.37 (C-5″), 101.77 (C-1″).

### Example 4

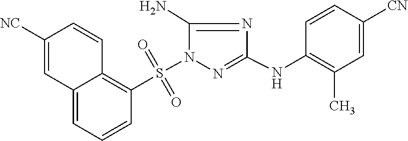 [SMILES: Cc1cc(C#N)ccc1Nc1nc(N)n(S(=O)(=O)c2cccc3cc(C#N)ccc23)n1]

### 5-[[5-amino-3-(4-cyano-2-methyl-anilino)-1,2,4-triazol-1-yl]sulfonyl]naphthalene-2-carbonitrile (Compound 4)

Yield 61%. Mass (EI), m/z (Irelat.(%)): 429.4558 [M]+ (54). C₂₁H₁₅N₇O₂S. ¹H NMR (500 MHz, DMSO) δ 9.12 (s, 1H, NH), 9.08 (d, J=9.1 Hz, 1H, HC(4′)), 8.74 (d, J=1.7 Hz, 1H, HC(1′)), 8.62 (d, J=7.2 Hz, 1H, HC(6′)), 8.48 (d, J=8.0 Hz, 1H, HC(8′)), 8.10 (dd, J=9.1, 1.9 Hz, 1H, HC(3′)), 7.93 (t, J=7.9 Hz, 1H, HC(7′)), 7.60 (d, J=8.2 Hz, 1H, HC(6″)), 7.52 (s, 2H, NH₂), 7.43 (dd, J=1.2, 8.2 Hz, 1H HC(5″), 7.40 (d, J=1.2 Hz, 1H, HC(2″)), 2.30 (s, 3H, CH₃). ¹³C NMR (50 MHz, DMSO) δ 158.71 (C-3), 156.43 (C-5), 141.43 (C-1′″), 136.28 (C-5′), 135.62 (C-1′), 133.86 (C-4a′), 134.71 (C-3″), 132.66 (C-8a′), 132.48 (C-3′), 131.21 (C-5″), 128.91 (C-2″), 128.72 (C-8′), 126.87 (C-6′), 126.45 (C-7′), 126.51 (C-4′), 120.21 (C-6″), 119.49 (CN—C2′), 116.12 (CN—C4″), 110.18 (C-2′), 105.60 (C-4″), 17.81 (CH₃).

### Example 5

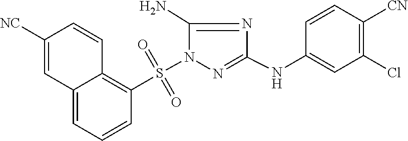 [SMILES: N#Cc1ccc2c(S(=O)(=O)n3nc(Nc4ccc(C#N)c(Cl)c4)nc3N)cccc2c1]

### 5-[[5-amino-3-(3-chloro-4-cyano-anilino)-1,2,4-triazol-1-yl]sulfonyl]naphthalene-2-carbonitrile (Compound 5)

Yield 74%. Mass (EI), m/z (Irelat.(%)): 449.8741 [M]+ (47). C₂₀H₁₂ClN₇₀₂S. ¹H NMR (500 MHz, DMSO) δ 10.07 (s, 1H, NH), 9.04 (d, J=9.0 Hz, 1H, HC(4′)), 8.79 (d, J=1.8 Hz, 1H, HC(1′), 8.65 (d, J=7.5 Hz, 1H, HC(6′)), 8.52 (d, J=8.2 Hz, 1H, HC(8′)), 8.11 (dd, J=9.0, 1.8 Hz, 1H, HC(3′)), 7.94 (t, J=7.9 Hz, 1H, HC(7′)), 7.78 (d, J=8.7 Hz, 1H, HC(5″)), 7.66 (s, 2H, NH₂), 7.64 (d, J=2.2 Hz, 1H, HC(2″)), 7.41 (dd, J=8.7, 2.2 Hz, 1H, HC(5″)). ¹³C NMR (126 MHz, DMSO) δ 158.15 (C-3), 156.40 (C-5), 145.60 (C-1′″), 137.12 (C-3″), 135.93 (C-5′), 135.47 (C-5″)), 134.91 (C-1′), 133.77 (C-6′), 132.79 (C-8a′), 132.01 (C-4a′), 129.17 (C-8′), 129.08 (C-3′), 126.64 (C-7′), 125.72 (C-4′), 118.11 (CN—C4″), 116.62 (CN—C2′), 116.26 (C-2″), 115.32 (C-6″), 110.16 (C-2′), 101.90 (C-4″).

### Example 6

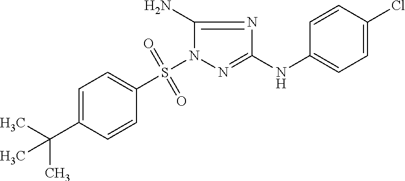 [SMILES: CC(C)(C)c1ccc(S(=O)(=O)n2nc(Nc3ccc(Cl)cc3)nc2N)cc1]

### 1-(4-tert-butylphenyl)sulfonyl-N3-(4-chlorophenyl)-1,2,4-triazole-3,5-diamine (Compound 6)

Yield 56%. Mass (EI), m/z (Irelat.(%)): 405.9026 [M]+ (33). C₁₈H₂₀ClN₅O₂S. ¹H NMR (500 MHz, DMSO) δ 9.36 (s, 1H, NH), 7.89 (d, J=8.2 Hz, 2H, HC(2′,6′)), 7.67 (d, J=8.2 Hz, 2H, HC(3′, 5′)), 7.49 (d, J=8.2, 2H, HC(2′, 6″), 7.35 (s, 2H, NH₂), 7.26 (d, J=8.2 Hz, 2H, HC(3″, 5″)), 1.27 (s, 9H, (CH₃)₃). ¹³C NMR (50 MHz, DMSO) δ 159.31 (C-3), 158.03 (C-4′), 157.02 (C-5), 139.68 (C-1″), 133.46 (C-1′), 128.35 (C-3″, 5″), 127.31 (C-2′, 6′), 126.50 (C-3′, 5′), 123.69 (C-4″), 118.12 (C-2′, 6″), 35.08 (CMe₃), 30.60 (CH₃).

### Example 7

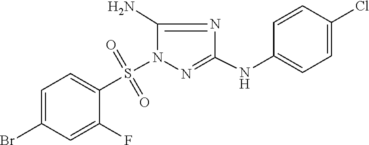 [SMILES: Nc1nc(Nc2ccc(Cl)cc2)nn1S(=O)(=O)c1ccc(Br)cc1F]

### 1-(4-bromo-2-fluoro-phenyl)sulfonyl-N3-(4-chlorophenyl)-1,2,4-triazole-3,5-diamine (Compound 7)

Yield 26%. Mass (EI), m/z (Irelat.(%)): 446.6828 [M]+ (31). C₁₄H₁₀BrClFN₅O₂S. ¹H NMR (200 MHz, DMSO) δ 9.35 (s, 1H), 7.96 (d, J=8.4 Hz, 1H, HC(6′), 7.88 (d, J=9.2 Hz, 1H, HC(3′)), 7.72 (d, J=8.4 Hz, 1H, HC(5′)), 7.41 (d, J=8.7 Hz, 2H, HC(3″, 5″)), 7.33 (s, 2H, NH₂), 7.21 (d, J=8.6 Hz, 2H, HC(2″, 6″). ¹³C NMR (50 MHz, DMSO) δ 159.46 (C-3), 158.03 (d, J=262 Hz, C-2′), 156.92 (C-5), 139.42 (C-1″), 131.96 (C-4′), 130.17 (d, J=9.3 Hz, C-6′), 128.66 (d, J=4.0 Hz, C-5′), 128.21 (C-3″, C-5″), 123.79 (C-4″), 123.53 (d, J=13.8 Hz, C-1′), 121.17 (d, J=24.3 Hz, C-3′), 118.05 (C-2, C-6).

### Example 8

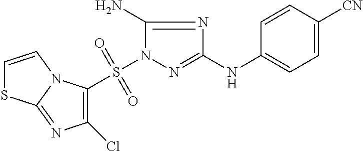 [SMILES: N#Cc1ccc(Nc2nc(N)n(S(=O)(=O)c3c(Cl)nc4sccn34)n2)cc1]

### 4-[[5-amino-1-(6-chloroimidazo[2,1-b]thiazol-5-yl)sulfonyl-1,2,4-triazol-3-yl]amino]-benzonitrile (Compound 8)

Yield 49%. Mass (EI), m/z (Irelat.(%)): 420.8587 [M]+ (37). C₁₄H₉ClN₈O₂S₂. ¹H NMR (200 MHz, DMSO) δ 9.86 (s, 1H, NH), 8.23 (d, J=4.4 Hz, 1H, HC(3′)), 7.78 (d, J=4.4 Hz, 1H, HC(2′)), 7.65-7.50 (m, 4H, HC(2″,3″,5″,6″)), 7.49 (s, 2H, NH₂). ¹³C NMR (50 MHz, DMSO) δ 159.27 (C-3), 156.75 (C-5), 151.69 (C-7a′), 144.41 (C-4″), 139.07 (C-6′), 132.83 (C-2″, C-6″), 120.85 (C-5′), 119.33 (C-3′), 117.67 (C-3″, C-5″), 116.69 (CN—C1″), 115.18 (C-2′), 101.63 (C-1″).

### Example 9

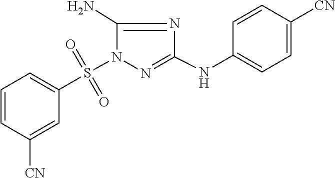 [SMILES: N#Cc1ccc(Nc2nc(N)n(S(=O)(=O)c3cccc(C#N)c3)n2)cc1]

### 3-[[5-amino-3-(4-cyanoanilino)-1,2,4-triazol-1-yl]sulfonyl]benzonitrile (Compound 9)

Yield 56%. Mass (EI), m/z (Irelat.(%)): 365.3705 [M]+ (46). C₁₆H₁₁N₇O₂S. ¹H NMR (200 MHz, DMSO) δ 9.88 (s, 1H, NH), 8.48 (s, 1H, HC(2′)), 8.25 (d, J=7.7 Hz, 2H, HC(4′), HC(6′)), 7.90 (t, J=7.7 Hz, 1H, HC(5′)), 7.72-7.56 (m, 4H, HC(2′, 3″, 5″, 6″), 7.53 (s, 2H, NH₂). ¹³C NMR (50 MHz, DMSO) δ 159.33 (C-3), 157.24 (C-5), 144.63 (C-1″), 138.41 (C-6′), 137.01 (C-3′), 133.07 (C-3′, 5″), 131.74 (C-2′), 131.25 (C-4′), 131.04 (C-5′), 119.43 (CN—C1′), 116.91 (CN—C₁″), 116.82 (C-2″, 6′), 113.03 (C-1′), 101.72 (C-4″).

### Example 10

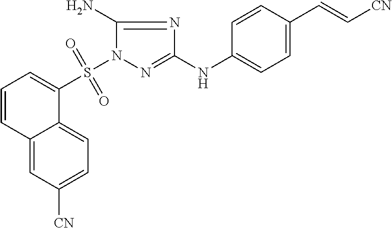 [SMILES: N#C/C=C/c1ccc(Nc2nc(N)n(S(=O)(=O)c3cccc4cc(C#N)ccc34)n2)cc1]

### 5-[[5-amino-3-[4-[(E)-2-cyanovinyl]anilino]-1,2,4-triazol-1-yl]sulfonyl]naphthalene-2-carbonitrile (Compound 10)

Yield 62%. Mass (EI), m/z (Irelat.(%)): 420.8587 [M]+ (82). C₂₂H₁₅N₇O₂S. ¹H NMR (200 MHz, DMSO) δ 9.57 (s, 1H, NH), 9.09 (d, J=9.0 Hz, 1H, HC(4′)), 8.78 (d, J=1.7 Hz, 1H, HC(1′)), 8.63 (dd, J=7.5, 1.2 Hz, 1H, HC(6′)), 8.50 (d, J=8.2 Hz, 1H, HC(8′)), 8.11 (dd, J=9.0, 1.8 Hz, 1H, HC(3′)), 7.93 (t, J=7.9 Hz, 1H, HC(7′)), 7.55 (s, 2H, NH₂), 7.50 (d, J=8.7 Hz, 2H, HC(2″, 6″)), 7.48 (d, J=16.5 Hz, 1H, (HC=)Ph), 7.40 (d, J=8.7 Hz, 2H, HC(3′, 5″)), 6.20 (d, J 15=16.5 Hz, 1H, (HC=)CN). ¹³C NMR (50 MHz, DMSO) δ 159.21 (C-3), 155.35 (C-5), 149.28 (PhC=), 144.54 (C-1″), 136.78 (C-5′), 135.52 (C-1′), 133.76 (C-4a′), 132.86 (C-8a′), 132.58 (C-3′), 128.72 (C-8′), 128.71 (C-3″, C-5″), 128.35 (C-4″), 126.87 (C-6′), 126.35 (C-7′), 126.41 (C-4′), 118.54 (CN—C2′), 118.12 (CN—C=), 116.71 (C-2″, C-6′), 110.28 (C-1′), 95.30 (=CCN).

### Example 11

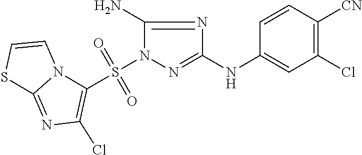 [SMILES: N#Cc1ccc(Nc2nc(N)n(S(=O)(=O)c3c(Cl)nc4sccn34)n2)cc1Cl]

### 4-[[5-amino-1-(6-chloroimidazo[2,1-b]thiazol-5-yl)sulfonyl-1,2,4-triazol-3-yl]amino]-2-chloro-benzonitrile (Compound 11)

Yield 76%. Mass (EI), m/z (Irelat.(%)): 455.3034 [M]+ (64). C₁₄H₈Cl₂N₈O₂S₂. ¹H NMR (200 MHz, DMSO) δ 10.16 (s, 1H, NH), 8.21 (d, J=4.4 Hz, 1H, HC(3′)), 7.85-7.69 (m, 3H, HC(5″), HC(2″), HC(2′)), 7.62 (s, 2H, NH₂), 7.40 (dd, J=8.7, 2.1 Hz, 1H, HC(6″)). ¹³C NMR (50 MHz, DMSO) δ 158.88 (C-3), 156.84 (C-5), 151.90 (C-7a′), 145.53 (C-4″), 139.41 (C-6′), 136.04 (C-2″), 134.74 (C-6″), 120.58 (C-5′), 118.12 (CN—C1′), 116.58 (C-3″), 116.43 (C-3′), 115.52 (C-2′), 115.00 (C-5″), 101.99 (C-1′).

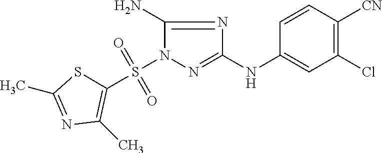 [SMILES: Cc1nc(C)c(S(=O)(=O)n2nc(Nc3ccc(C#N)c(Cl)c3)nc2N)s1]

### 4-[[5-amino-1-(2,4-dimethylthiazol-5-yl)sulfonyl-1,2,4-triazol-3-yl]amino]-2-chloro-benzonitrile (Compound 12)

Yield 79%. Mass (EI), m/z (Irelat.(%)): 409.8758 [M]+ (43). C₁₄H₁₂ClN₇O₂S₂. ¹H NMR (200 MHz, DMSO) δ 10.18 (s, 1H, NH), 7.89 (d, J=2.0 Hz, 1H, HC(3″)), 7.76 (d, J=8.7 Hz, 1H, HC(6″), 7.54 (s, 2H, NH₂), 7.46 (dd, J=8.7, 2.2 Hz, 1H, HC(5″), 2.67 (s, 6H, 2CH₃). ¹³C NMR (50 MHz, DMSO) δ 172.16 (C-2′), 159.27 (C-3), 158.90 (C-4′), 157.00 (C-5), 145.69 (C-4″), 136.09 (C-2″), 134.71 (C-6″), 124.61 (C-5′), 116.49 (C-3″, CN—C1″), 115.52 (C-5″), 102.02 (C-1″), 19.18 (CH₃—C₂′), 16.55 (CH₃—C₃′).

### Example 13

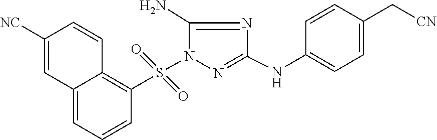 [SMILES: N#CCc1ccc(Nc2nc(N)n(S(=O)(=O)c3cccc4cc(C#N)ccc34)n2)cc1]

### 5-[[5-amino-3-[4-(cyanomethyl)anilino]-1,2,4-triazol-1-yl]sulfonyl]naphthalene-2-carbonitrile (Compound 13)

Yield 57%. Mass (EI), m/z (Irelat.(%)): 429.4558 [M]+ (89). C₂₁H₁₅N702S. ¹H NMR (200 MHz, DMSO) δ 9.18 (s, 1H, NH), 9.12 (d, J=9.1 Hz, 1H, HC(4′)), 8.73 (d, J=1.7 Hz, 1H, HC(1′)), 8.61 (d, J=7.5 Hz, 1H, HC(6′)), 8.47 (d, J=8.2 Hz, 1H, HC(8′)), 8.05 (dd, J=8.9, 1.8 Hz, 1H, HC(3′)), 7.91 (t, J=7.8 Hz, 1H, HC(7′)), 7.44 (s, 2H, NH₂), 7.37 (d, J=8.6 Hz, 2H, HC(2″), HC(6″), 7.18 (d, J=8.4 Hz, 2H, HC(3″), HC(5″)), 3.87 (s, 2H, CH₂CN). ¹³C NMR (50 MHz, DMSO) δ 159.09 (C-3), 156.30 (C-5), 139.86 (C-1″), 136.71 (C-5′), 135.20 (C-1′), 133.44 (C-4a′), 132.77 (C-6′), 132.37 (C-8a′), 129.31 (C-3′), 128.62 (C-8′), 128.25 (C-2″, 6″), 126.46 (C-7′), 126.34 (C-4′), 122.40 (C-4″), 119.18 (CN—CH₂), 118.06 (CN—C(2′)), 116.79 (C-3″, 5″), 110.03 (C-2′), 21.70 (CH₂).

### Example 14

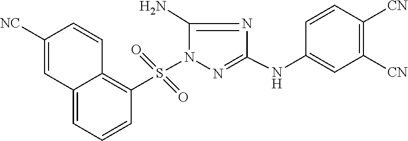 [SMILES: N#Cc1ccc2c(S(=O)(=O)n3nc(Nc4ccc(C#N)c(C#N)c4)nc3N)cccc2c1]

### 4-[[5-amino-1-[(6-cyano-1-naphthyl)sulfonyl]-1,2,4-triazol-3-yl]amino]phthalonitrile (Compound 14)

Yield 51%. Mass (EI), m/z (Irelat.(%)): 440.4387 [M]+ (73). C₂₁H₁₂N802S. ¹H NMR (200 MHz, DMSO) δ 10.27 (s, 1H, NH), 9.06 (d, J=9.1 Hz, 1H, HC(8′)), 8.78 (d, J=1.7 Hz, 1H, HC(5′)), 8.66 (d, J=7.5 Hz, 1H, HC(2′)), 8.52 (d, J=8.2 Hz, 1H, HC(4′), 8.10 (dd, J=9.1, 1.7 Hz, 1H, HC(7′)), 7.86-7.98 (m, 2H, HC(3′), HC(6″)), 7.88 (d, J=2.4 Hz, 1H, HC(3″)), 7.75 (dd, J=8.8, 2.3 Hz, 1H, HC(5″)), 7.66 (s, 2H, NH₂). ¹³C NMR (50 MHz, DMSO) δ 158.00 (C-3), 156.33 (C-5), 144.87 (C-4″), 137.17 (C-1′), 135.47 (C-6″), 134.84 (C-5′), 133.80 (C-8a′), 132.89 (C-4a′), 132.10 (C-2′), 129.25 (C-7′), 129.01 (C-4′), 126.60 (C-3′), 125.80 (C-3″), 125.76 (C-8′), 120.22 (C-5″), 118.06 (CN—C6′), 116.45 (CN—C1″), 116.06 (CN—C2″), 115.27 (C-2″), 110.30 (C-6′), 104.00 (C-1″).

### Example 15

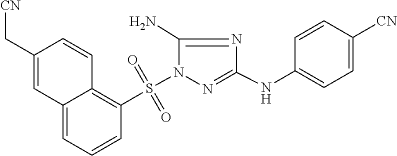 [SMILES: N#CCc1ccc2c(S(=O)(=O)n3nc(Nc4ccc(C#N)cc4)nc3N)cccc2c1]

### 4-[[5-amino-1-[[6-(cyanomethyl)-1-naphthyl]sulfonyl]-1,2,4-triazol-3-yl]amino]benzonitrile (Compound 15)

Yield 47%. Mass (EI), m/z (Irelat.(%)): 429.4558 [M]+ (62). C₂₁H₁₅N₇O₂S. ¹H NMR (500 MHz, DMSO) δ 9.79 (s, 1H, NH), 8.97 (d, J=8.9 Hz, 1H, HC(8′)), 8.49 (d, J=4.4 Hz, 1H, HC(2′)), 8.41 (d, J=9.7 Hz, 1H, HC(4′)), 8.08 (s, 1H, HC(5′)), 7.80 (d, J=8.9, 1H, HC(7′), 7.78 (t, J=7.7 Hz, 1H, HC(3′)), 7.66 (d, J=8.8 Hz, 2H, HC(2′, 6″)), 7.56 (s, 2H, NH₂), 7.52 (d, J=8.7 Hz, 2H, HC(3″, 5″)), 4.27 (s, 2H, CH₂). ¹³C NMR (126 MHz, DMSO) δ 158.28 (C-3), 156.32 (C-5), 144.55 (C-4″), 136.31 (C-1′), 133.79 (C-2, 6′), 131.80 (C-8a′), 131.48 (C-4a′), 129.99 (C-2′), 128.81 (C-4′), 127.50 (C-6′), 126.85 (C-5′), 125.01 (C-3′), 124.99 (C-7′), 123.35 (C-8′), 119.49 (CN(CH₂)), 118.68 (CNAr), 116.50 (C-3″, 5″), 101.51 (C-1″), 23.32 (CH₂).

### Example 16

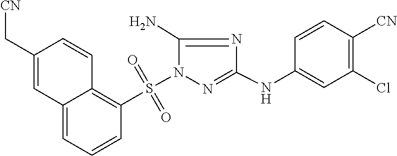 [SMILES: N#CCc1ccc2c(S(=O)(=O)n3nc(Nc4ccc(C#N)c(Cl)c4)nc3N)cccc2c1]

### 4-[[5-amino-1-[[6-(cyanomethyl)-1-naphthyl]sulfonyl]-1,2,4-triazol-3-yl]amino]-2-chloro-benzonitrile (Compound 16)

Yield 23%. Mass (EI), m/z (Irelat.(%)): 463.9005 [M]+ (81). C₂₁H₁₄ClN₇O₂S. ¹H NMR (500 MHz, DMSO) δ 10.04 (s, 1H), 8.91 (d, J=8.3 Hz, 1H, HC(8′)), 8.49 (dd, J=1.0, 7.3 Hz, 1H HC(2′)), 8.42 (d, J=8.2 Hz, 1H, HC(4′)), 8.10 (s, 1H, HC(5′)), 7.73-7.83 (m, 2H, HC(3′), HC(7′)), 7.76 (d, J=8.6 Hz, 1H, HC(6″)), 7.71 (d, J=2.1 Hz, 1H, HC(3″)), 7.63 (s, 2H, NH₂), 7.38 (dd, J=8.7, 2.2 Hz, 1H, HC(5″)), 4.27 (s, 2H, CH₂). ¹³C NMR (126 MHz, DMSO) δ 157.99 (C-3), 156.45 (C-5), 145.72 (C-4″), 136.43 (C-1′), 135.85 (C-2″), 134.84 (C-6″), 133.80 (C-8a′), 132.93 (C-2′), 131.31 (C-4a′), 128.90 (C-4′), 127.61 (C-6′), 126.80 (C-3′), 125.41 (C-5′), 125.02 (C-7′), 123.05 (C-8′), 118.63 (CN—CH₂), 116.65 (CN—C1″), 116.33 (C-5″), 116.22 (C-3″), 101.82 (C-1″), 22.35 (CH₂).

### Example 17

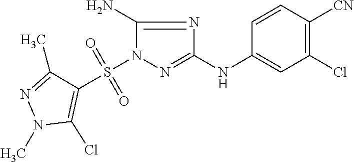 [SMILES: Cc1nn(C)c(Cl)c1S(=O)(=O)n1nc(Nc2ccc(C#N)c(Cl)c2)nc1N]

### 4-[[5-amino-1-(5-chloro-1,3-dimethyl-pyrazol-4-yl)sulfonyl-1,2,4-triazol-3-yl]amino]-2-chloro-benzonitrile (Compound 17)

Yield 59%. Mass (EI), m/z (Irelat.(%)): 429.4558 [M]+ (70). C₁₄H₁₂Cl₂N₈O₂S. ¹H NMR (200 MHz, DMSO) δ 10.13 (s, 1H, NH), 7.92 (d, J=2.4 Hz, 1H, HC(3″)), 7.74 (d, J=8.7 Hz, 1H, HC(6″)), 7.41 (dd, J=2.4, 8.7 Hz, 1H, HC(3″)), 7.39 (s, 2H, NH₂), 3.79 (s, 3H, NCH₃), 2.43 (s, 3H, CH₃—C(3′)). ¹³C NMR (50 MHz, DMSO) δ 158.21 (C-3), 156.27 (C-5), 148.81 (C-3′), 145.78 (C-4″), 136.07 (C-2″), 134.56 (C-6″), 130.89 (C-5′), 116.52 (C-3″), 116.31 (CN), 115.45 (C-5″), 111.85 (C-4′)), 101.75 (C-1″), 36.74 (NCH₃), 13.36 (CH₃—C₃′).

### Example 18

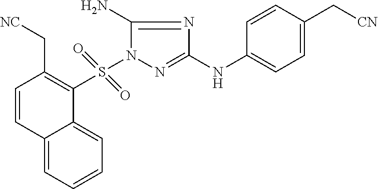 [SMILES: N#CCc1ccc(Nc2nc(N)n(S(=O)(=O)c3c(CC#N)ccc4ccccc34)n2)cc1]

### 2-[4-[[5-amino-1-[[6-(cyanomethyl)-2-naphthyl]sulfonyl]-1,2,4-triazol-3-yl]amino]phenyl]-acetonitrile (Compound 18)

Yield 35%. Mass (EI), m/z (Irelat.(%)): 429.4558 [M]+ (51). C₂₂H₁₇N702S. ¹H NMR (500 MHz, DMSO) δ 9.20 (s, 1H, NH), 8.96 (d, J=8.9 Hz, 1H, HC(8′)), 8.45 (d, J=4.4 Hz, 1H, HC(2′)), 8.39 (d, J=9.7 Hz, 1H, HC(4′)), 8.07 (s, 1H, HC(5′)), 7.77 (d, J=8.9, 1H, HC(7′), 7.75 (t, J=7.7 Hz, 1H, HC(3′)), 7.45 (s, 2H, NH₂), 7.37 (d, J=8.8 Hz, 2H, HC(2″, 6″)), 7.18 (d, J=8.7 Hz, 2H, HC(3″, 5″)), 4.26 (s, 2H, H₂C—C(6′)), 3.87 (s, 2H, H₂C—C(1″)). ¹³C NMR (126 MHz, DMSO) δ 159.01 (C-3), 156.47 (C-5), 139.97 (C-4″), 136.11 (C-1′), 133.80 (C-8a′), 132.89 (C-2′), 130.64 (C-4a′), 129.89 (C-4′), 128.29 (C-2″, 6″), 127.68 (C-6′), 126.99 (C-3′), 125.31 (C-5′), 124.95 (C-7′), 123.52 (C-8′), 119.46 (CN(CH₂—C(1″))), 118.68 (CN(CH₂—C(2′)), 116.79 (C-3″, 5″), 122.43 (C-1″), 22.35 (CH₂—C(6′)), 21.63 (CH₂C-1″).

### Example 19

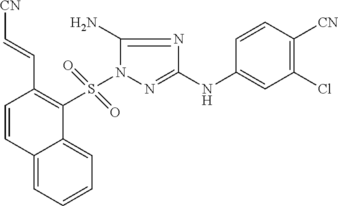 [SMILES: N#C/C=C/c1ccc2ccccc2c1S(=O)(=O)n1nc(Nc2ccc(C#N)c(Cl)c2)nc1N]

### 4-[[5-amino-1-[[7-[2-cyanovinyl]-1-naphthyl]sulfonyl]-1,2,4-triazol-3-yl]amino]-2-chloro-benzonitrile (Compound 19)

Yield 67%. Mass (EI), m/z (Irelat.(%)): 463.9005 [M]+ (44). C₂₂H₁₄ClN₇O₂S. ¹H NMR (500 MHz, DMSO) δ 10.05 (s, 1H, NH), 8.89 (d, J=1.6 Hz, 1H, HC(8′)), 8.54 (d, J=7.6 Hz, 1H, HC(2′)), 8.42 (d, J=8.2 Hz, 1H, HC(4′)), 8.20 (d, J=8.7 Hz, 1H, HC(5′)), 8.01 (dd, J=8.7, 1.6 Hz, 1H, HC(6′)), 7.85 (d, J=16.7 Hz, 1H, CH=(Ar)), 7.78 (s, 1H), 7.74 (s, 2H, NH₂), 7.63 (d, J=2.1 Hz, 1H, HC(3″)), 7.45 (dd, J=8.7, 2.2 Hz, 1H, HC(4″)), 6.64 (d, J=16.7 Hz, 1H, HC=(CN)). ¹³C NMR (50 MHz, DMSO) δ 159.23 (C-3), 156.51 (C-5), 149.75 (ArC=), 145.63 (C-1″), 136.21 (C-2′), 134.87 (C-5″), 133.57 (C-4a′), 132.42 (C-8′), 131.68 (C-8a′), 129.65 (C-5′), 128.37 (C-3″), 127.64 (C-4′), 125.54 (C-7′), 125.34 (C-3′), 125.54 (C-6′), 124.54 (C-1′), 122.42 (C-4″), 119.55 (CN(C=)), 118.10 (CN—C4″), 116.33 (C-6″), 116.25 (C-2′), 101.80 (C-4″).

### Example 20

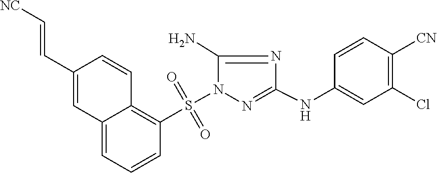 [SMILES: N#C/C=C/c1ccc2c(S(=O)(=O)n3nc(Nc4ccc(C#N)c(Cl)c4)nc3N)cccc2c1]

### 4-[[5-amino-1-[[6-[2-cyanovinyl]-1-naphthyl]sulfonyl]-1,2,4-triazol-3-yl]amino]-2-chloro-benzonitrile (compound 20)

Yield 24%. Mass (EI), m/z (Irelat.(%)): 463.9005 [M]+ (31). C₂₂H₁₄ClN₇O₂S. ¹H NMR (200 MHz, DMSO) δ 10.06 (s, 1H, NH), 8.88 (d, J=9.1 Hz, 1H, HC(8′)), 8.52 (d, J=7.5 Hz, 1H, HC(2′)), 8.42 (d, J=8.1 Hz, 1H, HC(4′)), 8.34 (d, J=1.7 Hz, 1H, HC(5′)), 8.10 (dd, J=9.2, 1.8 Hz, 1H, HC(7′)), 7.84 (d, J=16.6 Hz, 1H, HC=(CN)), 7.67 (d, J=2.1 Hz, 1H, HC(3″)), 7.65 (s, 2H, NH₂), 7.38 (dd, J=8.7, 2.1 Hz, 1H, HC(4″)), 6.69 (d, J=16.6 Hz, 1H, HC=(CN)). ¹³C NMR (50 MHz, DMSO) δ 159.13 (C-3), 156.28 (C-5), 151.12 (ArC=), 146.22 (C-4″), 136.88 (C-2″), 134.60 (C-6″), 133.57 (C-8a′), 132.50 (C-4a′), 131.90 (C-2′), 129.99 (C-7′), 128.69 (C-4′), 128.30 (C-6″), 126.14 (C-3′), 125.76 (C-3′), 124.04 (C-8′), 123.96 (C-6′), 123.87 (C-4′), 119.37 (CN(C=)), 119.12 (CN—C1″), 116.45 (C-3′), 116.21 (C-5″), 101.71 (C-1″).

### Example 21

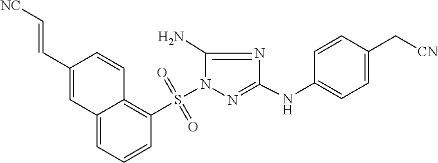 [SMILES: N#C/C=C/c1ccc2c(S(=O)(=O)n3nc(Nc4ccc(CC#N)cc4)nc3N)cccc2c1]

### 3-[5-[[5-amino-3-[4-(cyanomethyl)anilino]-1,2,4-triazol-1-yl]sulfonyl]-2-naphthyl]prop-2-enenitrile (Compound 21)

Yield 38%. Mass (EI), m/z (Irelat.(%)): 455.4931 [M]+ (55). C₂₃H₁₇N702S. ¹H NMR (500 MHz, DMSO) δ 9.20 (s, 1H, NH), 8.94 (d, J=9.1 Hz, 1H, HC(4′)), 8.48 (dd, J=7.6, 1.2 Hz, 1H, HC(6′)), 8.36 (d, J=8.2 Hz, 1H, HC(8′)), 8.29 (d, J=1.8 Hz, 1H, HC(1′)), 8.09 (dd, J=9.2, 1.9 Hz, 1H, HC(3′)), 7.83-7.78 (m, 2H, HC(7′), HC=(Ar)), 7.48 (s, 2H, NH₂), 7.34 (d, J=8.6 Hz, 2H, HC(2″, 6″)), 7.16 (d, J=8.7 Hz, 2H, HC(3″, 5″)), 6.68 (d, J=16.7 Hz, 1H, HC=(CN)), 3.88 (s, 2H, CH₂). ¹³C NMR (126 MHz, DMSO) δ 159.03 (C-3), 156.38 (C-5), 149.20 (ArC=), 139.90 (C-1″), 136.88 (C-5′), 134.60 (C-1′), 133.57 (C-4a′), 132.50 (C-6′), 131.90 (C-8a′), 129.99 (C-3′), 128.69 (C-8′), 128.29 (C-3″, C-5″), 126.14 (C-7′), 125.76 (C-4′), 123.96 (C-2′), 122.46 (C-4″), 119.37 (CN(CH₂)), 118.42 (CN(C=)), 116.86 (C-2″, C-6″), 99.01 (CN(C=)), 21.66 (CH₂).

### Example 22

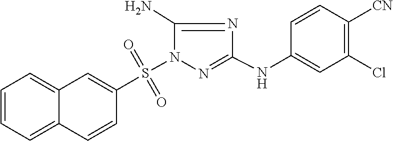 [SMILES: N#Cc1ccc(Nc2nc(N)n(S(=O)(=O)c3ccc4ccccc4c3)n2)cc1Cl]

### N3-(3,4-dichlorophenyl)-1-(1-naphthylsulfonyl)-1,2,4-triazole-3,5-diamine (Compound 22)

Yield 43%. Mass (EI), m/z (Irelat.(%)): 434.2997 [M]+ (18). C₁₈H₁₃Cl₂N₅O₂S. ¹H NMR (200 MHz, DMSO) δ 9.54 (s, 1H), 8.72 (d, J=2.0 Hz, 1H, HC(2″)), 8.21 (d, J=8.2 Hz, 1H, HC(2′)), 8.18 (d, J=8.5 Hz, 1H, HC(4′)), 8.06 (d, J=8.9 Hz, 1H, HC(8′), 7.90 (dd, J=8.7, 1.9 Hz, 1H, HC(5′)), 7.65-7.82 (m, 3H, HC(3′,5′,6′)), 7.48 (s, 2H, NH₂), 7.43 (d, J=8.8 Hz, 1H, HC(5″)), 7.36 (dd, J=8.8, 2.0 Hz, 1H, HC(6″)). ¹³C NMR (50 MHz, DMSO) δ 158.90 (C-3), 157.00 (C-5), 141.05 (C-1″), 135.36 (C-1′), 132.77 (C-3″), 131.16 (C-4a′), 130.65 (C-8a′), 130.04 (C-2′), 129.57 (C-5″), 129.27 (C-4′, C-5′), 127.79 (C-8′, C-6′), 127.70 (C-4″), 121.52 (C-3′), 121.12 (C-7′), 117.43 (C-6″), 116.54 (C-2″).

### Example 23

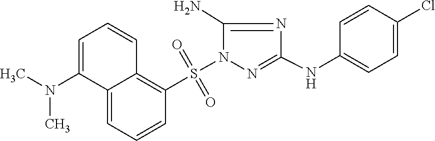 [SMILES: CN(C)c1cccc2c(S(=O)(=O)n3nc(Nc4ccc(Cl)cc4)nc3N)cccc12]

### N3-(4-chlorophenyl)-1-[[5-(dimethylamino)-1-naphthyl]sulfonyl]-1,2,4-triazole-3,5-diamine (Compound 23)

Yield 52%. Mass (EI), m/z (Irelat.(%)): 442.9228 [M]+ (54). C₂₀H₁₉ClN₆O₂S. ¹H NMR (200 MHz, DMSO) δ 9.28 (s, 1H, NH), 8.59 (d, J=8.2 Hz, 1H, HC(2′)), 8.55 (d, J=8.3 Hz, 1H, HC(4′)), 8.43 (d, J=7.3 Hz, 1H, HC(8′)), 7.72 (t, J=8.2 Hz, 1H, HC(3′)), 7.66 (t, J=8.1 Hz, 1H, HC(7′)), 7.41 (d, J=8.8 Hz, 2H, HC(3″, 5″)), 7.40 (s, 2H, NH₂), 7.23 (d, J=8.8 Hz, 2H, HC(2′, 6″)), 2.80 (s, 6H, N(CH₃)₂). ¹³C NMR (50 MHz, DMSO) δ 158.47 (C-3), 156.07 (C-5), 151.31 (C-5′), 139.49 (C-1′), 132.39 (C-1′), 131.96 (C-8a′), 130.57 (C-4′), 129.26 (C-2′), 128.99 (C-7′), 128.53 (C-4a′), 128.14 (C-3″, 5″), 123.72 (C-4″), 123.47 (C-3′), 118.83 (C-8′), 117.86 (C-2″, 6″), 115.58 (C-6′), 44.91 (N(CH₃)₂).

### Example 24

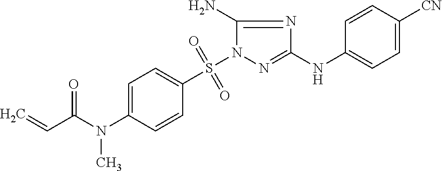 [SMILES: C=CC(=O)N(C)c1ccc(S(=O)(=O)n2nc(Nc3ccc(C#N)cc3)nc2N)cc1]

### N-[4-[[5-amino-3-(4-cyanoanilino)-1,2,4-triazol-1-yl]sulfonyl]phenyl]-N-methyl-prop-2-enamide (Compound 24)

Yield 47%. Mass (EI), m/z (Irelat.(%)): 423.4497 [M]+ (63). C₁₉H₁₇N₇O₃S. ¹H NMR (200 MHz, DMSO) δ 9.86 (s, 1H, NH), 8.03 (d, J=8.6 Hz, 2H, HC(3′,5′)), 7.65 (d, J=8.5 Hz, 2H, HC(3″, 5″)), 7.60 (d, J=8.5 Hz, 2H, HC2′, 6′)), 7.59 (d, J=8.6 Hz, 2H, HC(2″,6″)), 7.46 (s, 2H, NH₂), 6.18 (d, J=4.5 Hz, 1H, (Z)—HC=), 6.17 (d, J=7.8 Hz, 1H, (E)-HC=), 5.61 (dd, J=7.7, 4.8 Hz, 1H, HC═CO), 3.30 (s, 3H, NCH₃). ¹³C NMR (50 MHz, DMSO) δ 164.42 (C═O), 158.90 (C-3), 156.99 (C-5), 148.60 (C-1′), 144.69 (C-1″), 133.71 (C3″, 5″), 132.98 (C-4′), 128.67 (C-3′,5′), 128.55 (H2C=), 128.04 (CCO), 127.43 (C2′, 6′), 119.37 (CN), 116.64 (C-2′, 6′), 101.45 (C-4″), 36.50 (N(CH₃)₂).

### Example 25

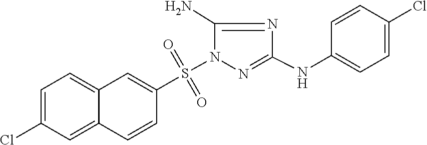 [SMILES: Nc1nc(Nc2ccc(Cl)cc2)nn1S(=O)(=O)c1ccc2cc(Cl)ccc2c1]

### 1-[(6-chloro-2-naphthyl)sulfonyl]-N3-(4-chlorophenyl)-1,2,4-triazole-3,5-diamine (Compound 25)

Yield 62%. Mass (EI), m/z (Irelat.(%)): 434.2997 [M]+ (71). C₁₈H₁₃Cl₂N₅O₂S. ¹H NMR (200 MHz, DMSO) δ 9.32 (s, 1H, NH), 8.77 (d, J=1.9 Hz, 1H, HC(1′)), 8.29 (d, J=8.7 Hz, 1H, HC(4′)), 8.19 (d, J=1.9 Hz, 1H, HC(5′)), 8.16 (d, J=9.2 Hz, 1H, HC(8′), 7.95 (dd, J=8.7, 2.0 Hz, 1H, HC(3′)), 7.71 (dd, J=8.8, 2.1 Hz, 1H, HC(7′)), 7.45 (d, J=8.9 Hz, 2H, HC(3′, 5″)), 7.41 (s, 2H, NH₂), 7.24 (d, J=8.9 Hz, 2H, HC(2″,6″)). ¹³C NMR (50 MHz, DMSO) δ 159.57 (C-3), 157.33 (C-5), 139.56 (C-1″), 135.80 (C4a′), 134.62 (C-2′), 133.47 (C-6′), 131.68 (C-8a′), 129.92 (C-8′), 129.49 (C-7′), 129.07 (C-3′), 128.65 (C-5), 128.34 (C-3″, 5″), 126.74 (C-4′), 123.73 (C-4″, 123.10 (C-1′), 118.15 (C-2″, 6″).

### Example 26

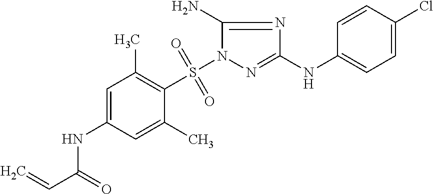 [SMILES: C=CC(=O)Nc1cc(C)c(S(=O)(=O)n2nc(Nc3ccc(Cl)cc3)nc2N)c(C)c1]

### N-[4-[[5-amino-3-(4-chloroanilino)-1,2,4-triazol-1-yl]sulfonyl]-3,5-dimethyl-phenyl]prop-2-enamide (Compound 26)

Yield 57%. Mass (EI), m/z (Irelat.(%)): 446.9115 [M]+ (84). C₁₉H₁₉ClN₆O₃S. ¹H NMR (500 MHz, DMSO) δ 10.42 (s, 1H, NHCO), 9.33 (s, 1H, NH), 8.29 (s, 1H, ?), 7.60 (s, 2H, HC(2′,6′)), 7.39 (d, J=8.9 Hz, 2H, HC(3″, 5″)), 7.25 (s, 2H, NH₂), 7.21 (d, J=8.9 Hz, 1H, HC(2″,6″)), 6.42 (dd, J=17.0, 10.1 Hz, 1H, (E)-HC=), 6.30 (dd, J=17.0, 2.0 Hz, 1H, (Z)—HC=), 5.81 (dd, J=10.1, 2.0 Hz, 1H, HCCO), 2.65 (s, 6H, 2CH₃). ¹³C NMR (126 MHz, DMSO) δ 163.74 (CO), 158.56 (C-3), 155.69 (C-5), 143.29 (C-1′), 141.82 (C-3′,5′), 139.78 (C-1″), 131.26 (=CCO), 128.25 (C-4′), 128.22 (C-3″, 5″), 128.08 (H₂C=), 123.45 (C-4″), 120.82 (C-2′, 6′), 117.87 (C-2″, 6″), 22.80 (2CH₃).

### Example 27

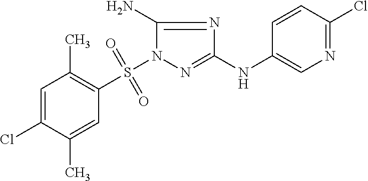 [SMILES: Cc1cc(S(=O)(=O)n2nc(Nc3ccc(Cl)nc3)nc2N)c(C)cc1Cl]

### 1-(4-chloro-2,5-dimethyl-phenyl)sulfonyl-N3-(6-chloro-3-pyridyl)-1,2,4-triazole-3,5-diamine (Compound 27)

Yield 61%. Mass (EI), m/z (Irelat.(%)): 413.2823 [M]+ (39). C₁₅H₁₄Cl₂N₆O₂S. ¹H NMR (500 MHz, DMSO) δ 9.61 (s, 1H, NH), 8.44 (d, J=2.9 Hz, 1H, HC(2″)), 8.05 (s, 1H, HC(6′)), 7.85 (dd, J=8.7, 2.9 Hz, 1H, HC(4″)), 7.55 (s, 1H, HC(3′)), 7.39 (s, 2H, NH₂), 7.32 (d, J=8.7 Hz, 1H, HC(5″)), 2.60 (s, 3H, CH₃—C(2′)), 2.39 (s, 3H, CH₃—C(5′)). ¹³C NMR (50 MHz,) δ 158.60 (C-3), 156.42 (C-5), 140.50 (C-6″), 139.74 (C-2″), 137.83 (C-2′), 137.47 (C-3″), 136.89 (C-4′), 134.38 (C-5′), 133.59 (C-1′), 132.86 (C-3′), 131.95 (C-6′), 126.52 (C-4′), 123.63 (C-5″), 19.19 (CH₃), 18.91 (CH₃).

### Example 28

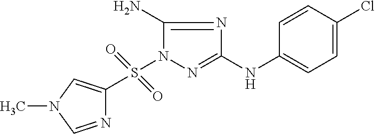 [SMILES: Cn1cnc(S(=O)(=O)n2nc(Nc3ccc(Cl)cc3)nc2N)c1]

### N3-(4-chlorophenyl)-1-(1-methylimidazol-4-yl)sulfonyl-1,2,4-triazole-3,5-diamine (Compound 28)

Yield 32%. Mass (EI), m/z (Irelat.(%)): 353.7884 [M]+ (45). C₁₂H₁₂ClN₇O₂S. ¹H NMR (300 MHz, DMSO) δ 9.28 (s, 1H, NH), 8.15 (s, 1H, HC(5′)), 7.81 (s, 1H, HC(2′)), 7.45 (d, J=8.8 Hz, 2H, HC(3″,5″)), 7.23 (d, J=8.6 Hz, 2H, HC(2″,6″)), 7.14 (s, 2H, NH₂), 3.72 (s, 3H, NCH₃). ¹³C NMR (50 MHz, DMSO) δ 158.87 (C-3), 157.17 (C-5), 140.45 (C-2′), 139.74 (C-1″), 135.13 (C-4′), 128.19 (C-3″,5″), 127.46 (C-5′), 123.40 (C-4″), 117.95 (C-2″, 6″), 33.67 (NCH₃).

### Example 29

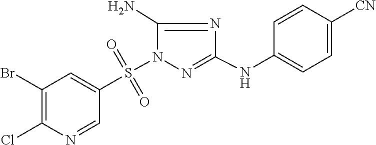 [SMILES: N#Cc1ccc(Nc2nc(N)n(S(=O)(=O)c3cnc(Cl)c(Br)c3)n2)cc1]

### 1-[(5-bromo-6-chloro-3-pyridyl)sulfonyl]-N3-(4-chlorophenyl)-1,2,4-triazole-3,5-diamine (Compound 29)

Yield 45%. Mass (EI), m/z (Irelat.(%)): 464.1252 [M]+ (78). C₁₃H₉BrCl₂N₆O₂S. ¹H NMR (500 MHz, DMSO) δ 9.43 (s, 1H, NH), 8.93 (d, J=2.2 Hz, 1H, HC(2′)), 8.72 (d, J=2.2 Hz, 1H, HC(4′)), 7.49 (d, J=8.9 Hz, 2H, HC(3″,5″), 7.48 (s, 2H, NH₂), 7.30 (d, J=8.9 Hz, 2H, HC(2″,6″). ¹³C NMR (75 MHz, DMSO) δ 159.12 (C-3), 156.67 (C-5), 153.78 (C-6′), 145.74 (C-2′), 139.74 (C-1″), 136.23 (C-4′), 131.45 (C-3′), 128.19 (C-3″,5″), 123.40 (C-4″), 118.46 (C-5′), 117.95 (C-2″, 6″).

### Example 30

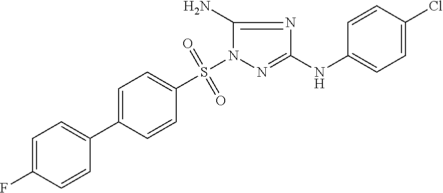 [SMILES: Nc1nc(Nc2ccc(Cl)cc2)nn1S(=O)(=O)c1ccc(-c2ccc(F)cc2)cc1]

### N3-(4-chlorophenyl)-1-[4-(4-fluorophenyl)phenyl]sulfonyl-1,2,4-triazole-3,5-diamine (Compound 30)

Yield 41%. Mass (EI), m/z (Irelat.(%)): 443.8827 [M]+ (65). C₂₀H₁₅ClFN₅O₂S. ¹H NMR (300 MHz, DMSO) δ 9.35 (s, 1H), 8.02 (d, J=8.5 Hz, 2H, HC(2′,6′)), 7.90 (d, J=8.5 Hz, 2H, HC(3′,5′)), 7.76 (dd, J=8.8, 5.5 Hz, 2H, HC(3′″,5′″)), 7.49 (d, J=8.9 Hz, 2H, HC(3″,5″)), 7.38 (s, 2H, NH₂), 7.28 (d, J=8.8 Hz, 2H, HC(2′″, 6′″)), 7.26 (d, J=8.9 Hz, 2H, HC(2″, 6″). ¹³C NMR (50 MHz, DMSO) δ 165.9 (d, J=98 Hz, (C-4′″)), 159.45 (C-3), 157.17 (C-5), 145.05 (C-4′), 139.53 (C-1″), 134.61 (C-1′), 134.41 (d, J=11 Hz, C-1′″), 129.40 (C-2′,6′), 129.22 (C-3″, 5″), 128.10 (d, J=38 Hz, C-2′,6′″)), 127.97 (C-3′,5′), 123.67 (C-4″), 118.06 (C-2″,6′), 115.80 (d, J=53 Hz, (C-3′,5″)).

### Example 31

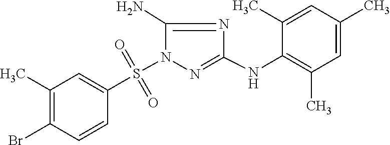 [SMILES: Cc1cc(C)c(Nc2nc(N)n(S(=O)(=O)c3ccc(Br)c(C)c3)n2)c(C)c1]

### 1-(4-bromo-3-methyl-phenyl)sulfonyl-N3-(2,4,6-trimethylphenyl)-1,2,4-triazole-3,5-diamine (Compound 31)

Yield 64%. Mass (EI), m/z (Irelat.(%)): 450.3539 [M]+ (71). C₁₈H₂₀BrN₅O₂S. ¹H NMR (200 MHz, DMSO) δ 7.86 (d, J=8.4 Hz, 1H, HC(5′)), 7.75 (d, J=2.5 Hz, 1H, HC(2′)), 7.73 (s, 1H, NH), 7.53 (dd, J=8.3, 2.4 Hz, 1H, HC(6′)), 7.18 (s, 2H, NH₂), 6.81 (s, 2H, HC(3′, 5′)), 2.43 (s, 3H, C(3′)—CH₃), 2.21 (s, 3H, C(4″)—CH₃), 1.93 (s, 6H, C(2″,6″)—CH₃). ¹³C NMR (50 MHz, DMSO) δ 162.60 (C-3), 158.69 (C-5), 139.01 (C-3′)), 135.26 (C-1″), 135.14 (C-1′), 134.63 (C-5′), 134.07 (C-6′), 133.16 (C-2″, 6″), 131.04 (C-4″), 129.28 (C-2′), 128.19 (C-3″,5″), 126.16 (C-4′), 22.40 (C(3′)—CH₃), 20.43 (C(4″)—CH₃), 17.61 (C(2″, 6″)-2CH₃).

### Example 32

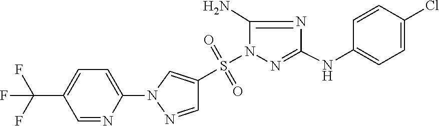 [SMILES: Nc1nc(Nc2ccc(Cl)cc2)nn1S(=O)(=O)c1cnn(-c2ccc(C(F)(F)F)cn2)c1]

### N3-(4-chlorophenyl)-1-[1-[5-(trifluoromethyl)-2-pyridyl]imidazol-4-yl]sulfonyl-1,2,4-triazole-3,5-diamine (Compound 32)

Yield 37%. Mass (EI), m/z (Irelat.(%)): 484.8438 [M]+ (65). C₁₇H₁₂CF₃N₈O₂S. ¹H NMR (200 MHz, DMSO) δ 9.39 (s, 1H, NH), 9.28 (s, 1H, HC(5′)), 8.93 (d, J=2.2 Hz, 1H, HC(6′″)), 8.44 (dd, J=8.7, 2.5 Hz, 1H, HC(4′″), 8.39 (s, 1H HC(2′)), 8.14 (d, J=8.7 Hz, 1H, HC(3′″)), 7.52 (d, J=8.8 Hz, 2H, HC(3″, 5″)), 7.39 (s, 2H, NH₂), 7.26 (d, J=8.8 Hz, 2H, HC(2″, 6″)). 15 ¹³C NMR (50 MHz, DMSO) δ 159.75 (C-3), 157.24 (C-5), 151.80 (C-2′″), 145.95 (q, J=5 Hz, C-6′″), 141.29 (C-2′), 139.50 (C-1′″), 137.69 (q, J=6 Hz, C-4′″), 130.25 (C-5′), 128.28 (C-3″, 5″), 126.2 (q, J=265 Hz, CF₃), 124.8 (q, J=30 Hz, C-5′″), 123.73 (C-4″), 121.01 (C-4′), 118.18 (C-2″, 6″), 113.22 (C-3′).

### Example 33

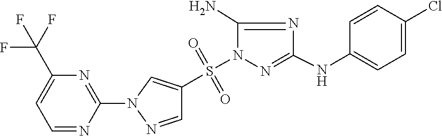 [SMILES: Nc1nc(Nc2ccc(Cl)cc2)nn1S(=O)(=O)c1cnn(-c2nccc(C(F)(F)F)n2)c1]

### N3-(4-chlorophenyl)-1-[1-[4-(trifluoromethyl)pyrimidin-2-yl]imidazol-4-yl]sulfonyl-1,2,4-triazole-3,5-diamine (Compound 33)

Yield 55%. Mass (EI), m/z (Irelat.(%)): 485.8319 [M]+ (16). C₁₆H₁₁ClF₃N₉O₂S. ¹H NMR (500 MHz, DMSO) δ 9.42 (s, 1H), 9.29 (s, 1H, HC(5′)), 9.28 (d, J=5.0 Hz, 1H, HC(6′″)), 8.39 (s, 1H, HC(2′)), 8.09 (d, J=5.0 Hz, 1H, HC(5″ ″)), 7.52 (d, J=8.9 Hz, 2H, HC(3″,5″)), 7.44 (s, 2H, NH₂), 7.28 (d, J=8.9 Hz, 2H, HC(2″,6″)). ¹³C NMR (126 MHz, DMSO) δ 163.39 (C-2′″), 159.89 (C-3), 157.41 (C-5), 155.4 (q, J=48 Hz, C-4′″), 154.50 (C-6′″), 141.58 (C-2′), 139.52 (C-1″), 132.66 (C-4′), 128.39 (C-3′, 5′), 123.82 (C-4′), 121.19 (C-5′), 119.84 (q, J=274 Hz, CF₃), 118.24 (C-2″, 6″), 116.88 (C-5′″).

### Example 34

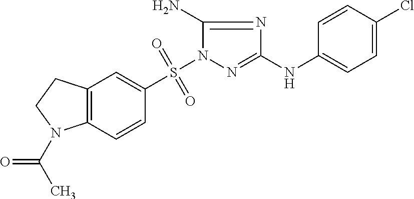 [SMILES: CC(=O)N1CCc2cc(S(=O)(=O)n3nc(Nc4ccc(Cl)cc4)nc3N)ccc21]

### 1-[5-[[5-amino-3-(4-chloroanilino)-1,2,4-triazol-1-yl]sulfonyl]indolin-1-yl]ethanone (Compound 34)

Yield 37%. Mass (EI), m/z (Irelat.(%)): 432.8849 [M]+ (76). C₁₈H₁₇ClN₆O₃S. ¹H NMR (300 MHz, DMSO) δ 9.32 (s, 1H, NH), 8.16 (d, J=8.6 Hz, 1H, HC(7′)), 7.79 (d, J=8.6 Hz, 1H, HC(6′), 7.77 (s, 1H, HC(4′)), 7.47 (d, J=8.9 Hz, 2H, HC(3″, 5″)), 7.28 (s, 2H, NH₂), 7.26 (d, J=8.9 Hz, 2H, HC(2′, 6″)), 4.13 (t, J=8.67 Hz, 2H, H₂C(2′)), 3.18 (t, J=8.7 Hz, 2H, H₂C(3′)), 2.17 (s, 3H, CH₃CO). ¹³C NMR (50 MHz, DMSO) δ 169.51 (CO), 159.25 (C-3), 157.07 (C-5), 148.09 (C-7a′), 139.62 (C-1″), 133.41 (C-3a′), 129.25 (C-5′), 128.25 (C-3″, 5″), 128.11 (C-4′), 123.90 (C-6′), 123.55 (C-4′), 117.99 (C-2″, 6″), 115.13 (C-7′), 48.64 (C-2′), 26.79 (C-3′), 23.91 (CH₃CO).

### Example 35

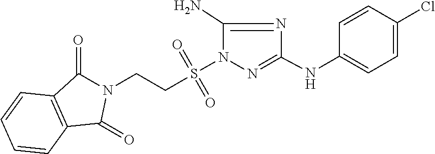 [SMILES: Nc1nc(Nc2ccc(Cl)cc2)nn1S(=O)(=O)CCN1C(=O)c2ccccc2C1=O]

### 2-[2-[[5-amino-3-(4-chloroanilino)-1,2,4-triazol-1-yl]sulfonyl]ethyl]isoindoline-1,3-dione (Compound 35)

Yield 64%. Mass (EI), m/z (Irelat.(%)): 446.8684 [M]+ (43). C₁₈H₁₅ClN₆O₄S. ¹H NMR (500 MHz, DMSO) δ 9.26 (s, 1H, NH), 7.72-7.77 (m, 4H, HC(4′, 5′, 6′, 7′), 7.43 (d, J=8.9 Hz, 2H, HC(3″, 5″)), 7.25 (d, J=8.9 Hz, 2H, HC(2′, 6″)), 7.11 (s, 2H, NH₂), 4.07-3.99 (m, 4H, 2CH₂). ¹³C NMR (126 MHz, DMSO) δ 167.03 (CO), 159.00 (C-3) 156.31 (C-5), 139.62 (C-1″), 134.23 (C-4′, 5′), 131.37 (C-3a′, 7a′), 128.23 (C-3″, 5″), 123.56 (C-4′, 7′), 122.93 (C-4″), 118.12 (C-2″, 6″), 49.30 (CS), 31.68 (CH₂N).

### Example 36

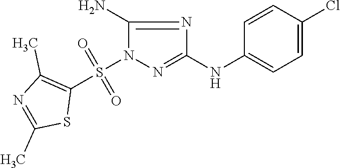 [SMILES: Cc1nc(C)c(S(=O)(=O)n2nc(Nc3ccc(Cl)cc3)nc2N)s1]

### N3-(4-chlorophenyl)-1-(2,4-dimethylthiazol-5-yl)sulfonyl-1,2,4-triazole-3,5-diamine (Compound 36)

Yield 57%. Mass (EI), m/z (Irelat.(%)): 384.8663 [M]+ (39). C₁₃H₁₃ClN₆O₂S₂. ¹H NMR (200 MHz, DMSO) δ 9.42 (s, 1H, NH), 7.51 (d, J=8.9 Hz, 2H, HC(3″, 5″)), 7.39 (s, 2H, NH₂), 7.26 (d, J=8.9 Hz, 2H, HC(2″, 6″)), 2.65 (d, J=1.8 Hz, 6H, 2CH₃). ¹³C NMR (50 MHz, DMSO) δ 171.73 (C-2′), 159.79 (C-3), 158.88 (C-4′), 157.15 (C-5), 139.41 (C-1″), 128.25 (C-3″, 5″), 124.61 (C-5′), 123.85 (C-4″), 118.09 (C-2″, 6″), 19.09 (CH₃—C(2′)), 16.54 (CH₃—C(4′)).

### Example 37

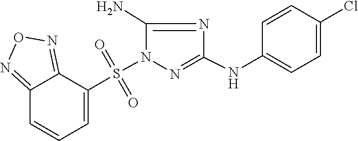 [SMILES: Nc1nc(Nc2ccc(Cl)cc2)nn1S(=O)(=O)c1cccc2nonc12]

### 1-(2,1,3-benzoxadiazol-4-ylsulfonyl)-N3-(4-chlorophenyl)-1,2,4-triazole-3,5-diamine (Compound 37)

Yield 24%. Mass (EI), m/z (Irelat.(%)): 391.7933 [M]+ (17). C₁₄H₁₀ClN₇O₃S. ¹H NMR (500 MHz, DMSO) δ 9.36 (s, 1H), 8.49 (d, J=9.0 Hz, 1H), 8.44 (d, J=6.8 Hz, 1H), 7.85 (dd, J=9.1, 6.9 Hz, 1H), 7.50 (s, 2H), 7.35 (d, J=8.9 Hz, 1H), 7.20 (d, J=8.9 Hz, 1H). ¹³C NMR (126 MHz, DMSO) δ 159.51 (C-3), 157.14 (C-5), 149.22 (C-3a′), 143.80 (C-7a′), 139.38 (C-1″), 137.49 (C-6′), 131.52 (C-5′), 128.27 (C-3″, 5″), 124.58 (C-4′), 123.77 (C-4′), 123.69 (C-7′), 118.09 (C-2″, 6″).

### Example 38

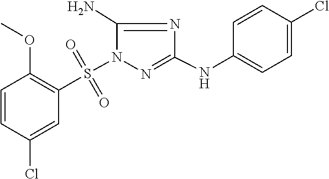 [SMILES: COc1ccc(Cl)cc1S(=O)(=O)n1nc(Nc2ccc(Cl)cc2)nc1N]

### 1-(5-chloro-2-methoxy-phenyl)sulfonyl-N3-(4-chlorophenyl)-1,2,4-triazole-3,5-diamine (Compound 38)

Yield 40%. Mass (EI), m/z (Irelat.(%)): 414.2670 [M]+ (65). C₁₅H₁₃Cl₂N₅O₃S. ¹H NMR (200 MHz, DMSO) δ 9.33 (s, 1H, NH), 7.89 (d, J=2.6 Hz, 1H, HC(6′), 7.76 (dd, J=9.0, 2.6 Hz, 1H, HC(4′)), 7.36 (d, J=9.1 Hz, 2H. HC(3′, 5″)), 7.28 (d, J=9.0 Hz, 1H, HC(3′)), 7.22 (s, 2H, NH₂), 7.20 (d, J=9.1 Hz, 2H, HC(2″, 6″)), 3.82 (s, 3H, OCH₃). ¹³C NMR (50 MHz, DMSO) δ 158.97 (C-3), 157.95 (C-2′), 155.79 (C-5), 139.66 (C-1′″), 136.22 (C-4′), 129.56 (C-5′), 128.22 (C-3″, 5″), 125.73 (C-6′), 124.14 (C-1′), 123.54 (C-4″), 117.91 (C-2′, 6′), 115.33 (C-3′), 56.62 (OCH₃).

### Example 39

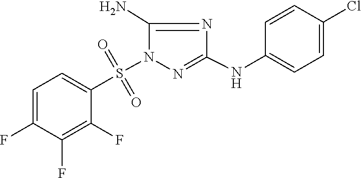 [SMILES: Nc1nc(Nc2ccc(Cl)cc2)nn1S(=O)(=O)c1ccc(F)c(F)c1F]

### N3-(4-chlorophenyl)-1-(2,3,4-trifluorophenyl)sulfonyl-1,2,4-triazole-3,5-diamine (Compound 39)

Yield 38%. Mass (EI), m/z (Irelat.(%)): 403.7677 [M]+ (73). C₁₄H₉ClF₃N₅O₂S. ¹H NMR (200 MHz, DMSO) δ 9.44 (s, 1H, NH), 8.02-7.83 (m, J=2.3, 5.6, 8.1, 9.3 Hz, 1H, HC(6′)), 7.62 (tdd, J=9.3, 6.7, 2.1 Hz, 1H, HC(5′)), 7.45 (s, 2H, NH₂), 7.43 (d, J=8.7 Hz, 2H, HC(3″, 5″)), 7.23 (d, J=8.8 Hz, 2H, HC(2′, 6″)). ¹³C NMR (50 MHz, DMSO) δ 159.73 (C-3), 156.94 (C-5), 154.0 (ddd, J=152, 8.0, 3.0 Hz, (C-4′)), 148.4 (ddd, J=4.6, 12.0, 146 Hz, (C-2′)), 140.1 (dt, J=255, 15 Hz, (C-3′)), 139.47 (C-1″), 128.31 (C-3″, 5″), 125.83 (dd, J=4.6, 9.0 Hz, C-6′), 123.92 (C-4″), 121.90 (dd, J=3.0, 12.0 Hz, C-1′), 117.59 (C-2″, 6″), 113.90 (dd, J=3.0, 19.0 Hz, C-5′).

### Example 40

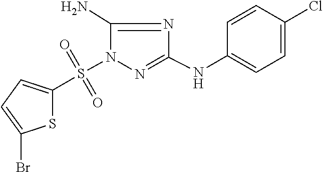 [SMILES: Nc1nc(Nc2ccc(Cl)cc2)nn1S(=O)(=O)c1ccc(Br)s1]

### 1-[(5-bromo-2-thienyl)sulfonyl]-N3-(4-chlorophenyl)-1,2,4-triazole-3,5-diamine (Compound 40)

Yield 47%. Mass (EI), m/z (Irelat.(%)): 434.7211 [M]+ (24). C₁₂H₉BrClN₅O₂S₂. ¹H NMR (200 MHz, DMSO) δ 9.47 (s, 1H, NH), 7.72 (d, J=4.1 Hz, 1H, HC(3′)), 7.53 (d, J=9.0 Hz, 2H, HC(3′, 5″)), 7.44 (s, 2H, NH₂), 7.42 (d, J=4.1 Hz, 1H, HC(4′)), 7.29 (d, J=9.0 Hz, 2H, HC(2″, 6″)). ¹³C NMR (50 MHz, DMSO) δ 160.18 (C-3), 157.61 (C-5), 139.41 (C-1″), 135.38 (C-2′), 135.26 (C-3′), 131.68 (C-4′), 128.37 (C-3″, 5″), 123.97 (C-4″), 122.58 (C-5′), 118.28 (C-2′, 6″).

### Example 41

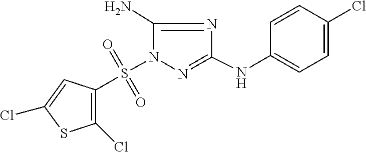 [SMILES: Nc1nc(Nc2ccc(Cl)cc2)nn1S(=O)(=O)c1cc(Cl)sc1Cl]

### N3-(4-chlorophenyl)-1-[(2,5-dichloro-3-thienyl)sulfonyl]-1,2,4-triazole-3,5-diamine (Compound 41)

Yield 34%. Mass (EI), m/z (Irelat.(%)): 424.7145 [M]+ (74). C₁₂H₈C₁₃N₅O₂S₂. ¹H NMR (200 MHz, DMSO) δ 9.47 (s, 1H, NH), 7.53 (s, 1H, HC(4′), 7.50 (d, J=8.8 Hz, 2H, HC(3″, 5″)), 7.42 (s, 2H, NH₂), 7.25 (d, J=8.7 Hz, 2H, HC(2″, 6″)). ¹³C NMR (50 MHz, DMSO) δ 159.69 (C-3), 156.96 (C-5), 139.47 (C-1″), 133.53 (C-3′), 131.86 (C-2′), 128.34 (C-3″, 5″), 127.28 (C-5′), 126.43 (C-4′), 123.88 (C-4″), 118.21 (C-2″, 6″).

### Example 42

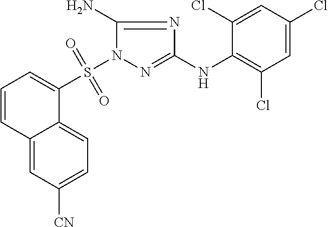 [SMILES: N#Cc1ccc2c(S(=O)(=O)n3nc(Nc4c(Cl)cc(Cl)cc4Cl)nc3N)cccc2c1]

### 5-[[5-amino-3-(2,4,6-trichloroanilino)-1,2,4-triazol-1-yl]sulfonyl]naphthalene-2-carbonitrile (Compound 42)

Yield 54%. Mass (EI), m/z (Irelat.(%)): 493.7540 [M]+ (39). C₁₉H₁₁C₁₃N₆O₂S. ¹H NMR (200 MHz, DMSO) δ 8.81 (s, 1H, NH), 8.77 (d, J=8.10 Hz, 1H, HC(4′) 8.54 (s, 1H, HC(1′)), 8.49 (d, J=7.9 Hz, 1H, HC(8′)), 7.90 (m, 2H, HC(7′,3′), 7.55 (s, 2H, HC(3″, 5″)), 7.45 (s, 2H, NH₂). ¹³C NMR (50 MHz, DMSO) δ 158.41 (C-3), 156.23 (C-5), 137.39 (C-1″), 136.58 (C-5′), 133.76 (C-4a′), 133.01 (C-3′, C-5″), 131.16 (C-8a′), 131.98 (C-3′), 129.10 (C-2″, 6″)), 128.72 (C-8′), 128.12 (C-3″,5″), 127.15 (C-4″), 126.87 (C-6′), 126.65 (C-7′), 126.21 (C-4′), 119.29 (CN), 110.08 (C-1′).

### Example 43

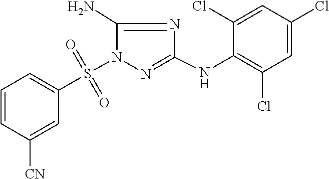 [SMILES: N#Cc1cccc(S(=O)(=O)n2nc(Nc3c(Cl)cc(Cl)cc3Cl)nc2N)c1]

### 3-[[5-amino-3-(2,4,6-trichloroanilino)-1,2,4-triazol-1-yl]sulfonyl]benzonitrile (Compound 43)

Yield 76%. Mass (EI), m/z (Irelat.(%)): 443.6953 [M]+ (57). C₁₅H₉Cl₃N₆O₂S. ¹H NMR (200 MHz,) δ 8.70 (s, 1H, NH), 8.26 (t, J=2.1 Hz, 1H, HC(2′)), 8.24 (d, J=2.1 Hz, 1H, HC(6′), 8.13 (dt, J=8.2, 1.5 Hz, 1H, HC(3′)), 7.88 (t, J=8.1 Hz, 1H, HC(5′)), 7.62 (s, 2H, HC(3″, 5″)), 7.41 (s, 2H, NH₂). ¹³C NMR (50 MHz,) δ 161.63 (C-3), 158.75 (C-5), 137.93 (C-1″), 136.89 (C-6′), 134.71 (C-2′), 133.95 (C-3′), 131.58 (C-4′), 131.32 (C-5′), 130.94 (C-2′, 6)), 128.15 (C-3, 5″, 4′), 116.89 (CN), 112.77 (C-1′).

### Example 44

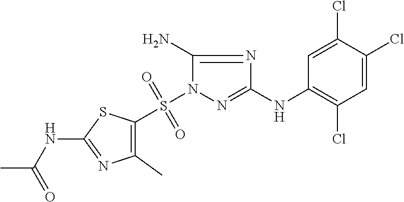 [SMILES: CC(=O)Nc1nc(C)c(S(=O)(=O)n2nc(Nc3cc(Cl)c(Cl)cc3Cl)nc2N)s1]

### N-[5-[[5-amino-3-(2,4,5-trichloroanilino)-1,2,4-triazol-1-yl]sulfonyl]-4-methyl-thiazol-2-yl]acetamide (Compound 44)

Yield 48%. Mass (EI), m/z (Irelat.(%)): 496.7806 [M]+ (53). C₁₄H₁₂Cl₃N₇O₃S₂. ¹H NMR (200 MHz, DMSO) δ 12.79 (s, 1H, CONH), 8.45 (s, 1H, NH), 8.29 (s, 1H, HC(6′)), 7.70 (s, 1H, HC(3′)), 7.45 (s, 2H, NH₂), 2.62 (s, 3H, CH₃Ar), 2.18 (s, 3H, CH₃CO). ¹³C NMR (50 MHz, DMSO) δ 169.70 (CO), 161.42 (C-2′), 158.85 (C-3), 156.99 (C-4′), 156.87 (C-5), 136.71 (C-1″), 130.01 (C-3″), 129.92 (C-5″), 122.97 (C-4″), 120.79 (C-2″), 120.13 (C-6′), 116.95 (C-5′), 22.21 (CH₃CO), 16.67 (CH₃—C-4′).

### Example 45

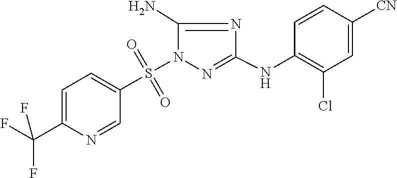 [SMILES: N#Cc1ccc(Nc2nc(N)n(S(=O)(=O)c3ccc(C(F)(F)F)nc3)n2)c(Cl)c1]

### 4-[[5-amino-1-[[6-(trifluoromethyl)-3-pyridyl]sulfonyl]-1,2,4-triazol-3-yl]amino]-3-chloro-benzonitrile (Compound 45)

Yield 41%. Mass (EI), m/z (Irelat.(%)): 443.7918 [M]+ (68). C₁₅H₉ClF₃N₇O₂S. ¹H NMR (200 MHz, DMSO) δ 9.33 (d, J=2.2 Hz, 1H, HC(2′)), 8.73 (s, 1H, NH), 8.68 (dd, J=8.4, 2.5 Hz, 1H, HC(4′)), 8.25 (d, J=8.4 Hz, 1H, HC(5′)), 8.16 (d, J=8.7 Hz, 1H, HC(5″)), 7.92 (d, J=1.9 Hz, 1H, HC(2″)), 7.75 (dd, J=8.9, 2.2 Hz, 2H, HC(6″)), 7.70 (s, 2H, NH₂). ¹³C NMR (50 MHz, DMSO) δ 159.18 (C-3), 157.21 (C-5), 151.19 (q, J=74 Hz, C(6′), 148.17 (C-2′), 140.83 (C-4″), 138.42 (C-4′), 135.56 (C-3′), 132.77 (C-2″), 131.89 (C-6″), 122.06 (C-5′), 121.8 (q, J=260 Hz, CF₃), 121.34 (C-3″), 119.25 (C-5″), 117.82 (CN), 103.81 (C-1″).

### Example 46

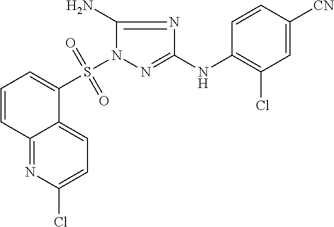 [SMILES: N#Cc1ccc(Nc2nc(N)n(S(=O)(=O)c3cccc4nc(Cl)ccc34)n2)c(Cl)c1]

### 4-[[5-amino-1-[(2-chloro-5-quinolyl)sulfonyl]-1,2,4-triazol-3-yl]amino]-3-chloro-benzonitrile (Compound 46)

Yield 33%. Mass (EI), m/z (Irelat.(%)): 460.2973 [M]+ (69). C₁₈H₁₁C₁₂N₇O₂S. 1H NMR (200 MHz, DMSO) δ 8.63 (d, J=8.4 Hz, 1H, HC(6′)), 8.62 (d, J=8.9 Hz, 1H, HC(8′)), 8.49 (s, 1H, NH), 8.50 (d, J=8.7 Hz, 1H, HC(3′)), 7.91 (t, J=7.8, 1H, HC(7′)), 7.84 (d, J=8.4, 1H, HC(4′), 7.81 (d, J=2.0 Hz, 1H, HC(2″)), 7.78 (d, J=8.7 Hz, 1H, HC(5″)), 7.58 (dd, J=8.7, 2.0 Hz, 1H, HC(6″)), 7.44 (s, 2H, NH₂). ¹³C NMR (50 MHz, DMSO) δ 157.85 (C-3), 157.66 (C-5), 151.69 (C-2′), 142.16 (C-8a′), 141.23 (C-5′), 140.75 (C-4″), 135.92 (C-4′), 134.24 (C-7′), 132.68 (C-2″), 132.41 (C-6′), 131.62 (C-6″), 127.31 (C-4a′), 126.37 (C-8′), 124.22 (C-3′), 120.73 (C-3″), 118.27 (C-5″), 117.91 (CN), 103.06 (C-1″).

### Example 47

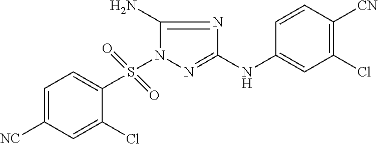 [SMILES: N#Cc1ccc(S(=O)(=O)n2nc(Nc3ccc(C#N)c(Cl)c3)nc2N)c(Cl)c1]

### 4-[[5-amino-1-(2-chloro-4-cyano-phenyl)sulfonyl-1,2,4-triazol-3-yl]amino]-3-chloro-benzonitrile (Compound 47)

Yield 45%. Mass (EI), m/z (Irelat.(%)): 434.2600 [M]+ (47). C₁₆H₉Cl₂N₇O₂S. ¹H NMR (200 MHz, DMSO) δ 10.16 (s, 1H), 8.38 (d, J=1.6 Hz, 1H), 8.34 (s, 1H, NH), 8.16 (dd, J=8.3, 1.6 Hz, 1H), 7.70 (d, J=8.8 Hz, 1H, HC(5″)), 7.66 (d, J=2.2 Hz, 1H, HC(2″), 7.59 (s, 2H, NH₂), 7.35 (dd, J=8.7, 2.1 Hz, 1H, HC(6″)). ¹³C NMR (50 MHz, DMSO) δ 158.42 (C-3), 157.03 (C-5), 145.71 (C-4″), 138.14 (C-1′), 135.98, 135.71, 134.70, 132.64, 132.34 (C-2″), 132.04 (C-6″), 118.46 (C-5″), 116.49 (C-3″), 116.36 (CN—C1′″), 115.94, 115.39 (C-5′), 101.90 (C-1″).

### Example 48

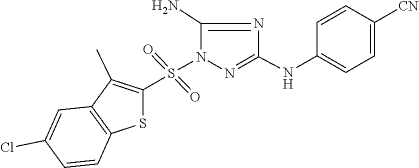 [SMILES: Cc1c(S(=O)(=O)n2nc(Nc3ccc(C#N)cc3)nc2N)sc2ccc(Cl)cc12]

### 4-[[5-amino-1-(5-chloro-3-methyl-benzothiophen-2-yl)sulfonyl-1,2,4-triazol-3-yl]amino]benzonitrile (Compound 48)

Yield 53%. Mass (EI), m/z (Irelat.(%)): 444.9198 [M]+ (32). C₁₈H₁₃ClN₆O₂S₂. ¹H NMR (200 MHz, DMSO) δ 9.89 (s, 1H, NH), 8.12 (d, J=9.0 Hz, 1H, HC(7′)), 8.09 (d, J=2.5 Hz, 1H, HC(4′)), 7.61 (m, 4H, HC(2″,3″,5″,6″), 7.60 (d, J=2.5, 9.0 Hz, HC(6′), 7.55 (s, 2H, NH₂), 2.79 (s, 3H, CH₃). ¹³C NMR (50 MHz,) δ 159.15 (C-3), 156.96 (C-5), 144.53 (C-4″), 140.86 (C-3a′), 139.75 (C-7a′), 138.04 (C-2′), 132.92 (C-2″, 6″), 131.95 (C-3′), 130.89 (C-5′), 128.61 (C-6′), 124.73 (C-4′), 124.07 (C-7′), 119.34 (CN), 116.67 (C-3″, 5′)), 101.59 (C-4″), 12.69 (CH₃).

### Example 49

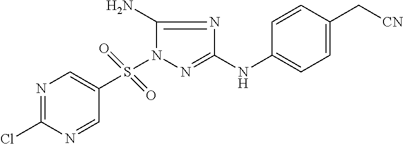 [SMILES: N#CCc1ccc(Nc2nc(N)n(S(=O)(=O)c3cnc(Cl)nc3)n2)cc1]

### 2-[4-[[5-amino-1-(2-chloropyrimidin-5-yl)sulfonyl-1,2,4-triazol-3-yl]amino]phenyl]-acetonitrile (Compound 49)

Yield 45%. Mass (EI), m/z (Irelat.(%)): 390.8086 [M]+ (61). C₁₄H₁₁ClN₈O₂S. ¹H NMR (200 MHz, DMSO) δ 9.36 (s, 1H, NH), 8.77 (s, 2H, HC(4′,6′), 7.52 (s, 2H, NH₂), 7.50 (d, J=8.5 Hz, 2H, HC(2″, 6″), 7.22 (d, J=8.3 Hz, 2H, HC(3″, 5″)), 3.89 (s, 2H, CH₂). ¹³C NMR (50 MHz, DMSO) δ 159.97 (C-3), 159.13 (C-2′), 157.21 (C-5), 148.32 (C-4′, 6′), 140.03 (C-4″), 128.40 (C-3″, 5″), 122.52 (C-1″), 122.2 (C-1′), 119.23 (CN), 117.15 (C-2′, 6″), 21.73 (CH₂).

### Example 50

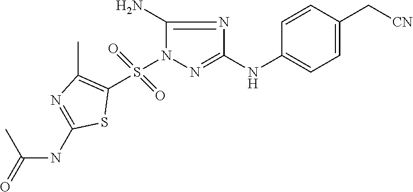 [SMILES: CC(=O)Nc1nc(C)c(S(=O)(=O)n2nc(Nc3ccc(CC#N)cc3)nc2N)s1]

### N-[5-[[5-amino-3-[4-(cyanomethyl)anilino]-1,2,4-triazol-1-yl]sulfonyl]-4-methyl-thiazol-2-yl]acetamide (Compound 50)

Yield 19%. Mass (EI), m/z (Irelat.(%)): 432.4824 [M]+ (14). C₁₆H₁₆N₈O₃S₂. ¹H NMR (200 MHz, DMSO) δ 12.74 (s, 1H, CONH), 9.31 (s, 1H, NH), 7.51 (d, J=8.5 Hz, 2H, HC(3″, 5″)), 7.33 (s, 2H, NH₂), 7.20 (d, J=8.3 Hz, 2H, HC(2″, 6″)), 3.89 (s, 2H, CH₂), 2.61 (s, 3H, CH₃Ar), 2.17 (s, 3H, CH₃CO). ¹³C NMR (50 MHz, DMSO) δ 169.67 (CO), 161.12 (C-2′), 159.72 (C-3), 157.06 (C-5), 156.42 (C-3′), 140.05 (C-4′), 128.22 (C-3′, 5′), 122.37 (C-1″), 119.21 (CN), 117.15 (C-1′), 116.91 (C-2″, 6″), 22.22 (MeCO), 21.64 (CH₂), 16.70 (CH₃—C-4′).

### Example 51

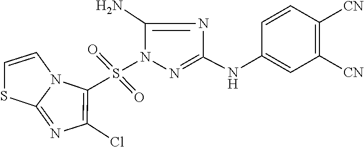 [SMILES: N#Cc1ccc(Nc2nc(N)n(S(=O)(=O)c3c(Cl)nc4sccn34)n2)cc1C#N]

### 4-[[5-amino-1-(6-chloroimidazo[2,1-b]thiazol-5-yl)sulfonyl-1,2,4-triazol-3-yl]amino]-phthalonitrile (Compound 51)

Yield 67%. Mass (EI), m/z (Irelat.(%)): 445.8682 [M]+ (16). C₁₅H₈ClN₉O₂S₂. ¹H NMR (200 MHz, DMSO) δ 10.37 (s, 1H, NH), 8.24 (d, J=4.5 Hz, 1H, HC(3′)), 8.03 (d, J=2.2 Hz, 1H, HC(3″), 7.91 (d, J=8.2 Hz, 1H, HC(6″), 7.80-7.69 (m, 2H, HC(2′), HC(5″)), 7.62 (s, 2H, NH₂). ¹³C NMR (50 MHz, DMSO) δ 158.66 (C-3), 156.69 (C-5), 151.87 (C-7a′), 144.78 (C-4″), 139.50 (C-6′), 134.62 (C-6″), 120.53 (C-3″), 120.42 (C-5″), 120.34 (C-5′), 118.00 (C-3′), 116.33 (C-2′), 115.88 (CN), 115.24 (CN), 114.9 (C-2″), 104.05 (C-1′″).

### Example 52

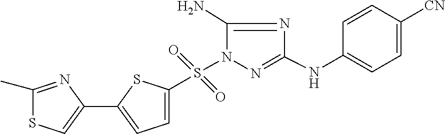 [SMILES: Cc1nc(-c2ccc(S(=O)(=O)n3nc(Nc4ccc(C#N)cc4)nc3N)s2)cs1]

### 4-[[5-amino-1-(2-methylthiazol-4-yl)sulfonyl-1,2,4-triazol-3-yl]amino]benzonitrile (Compound 52)

Yield 37%. Mass (EI), m/z (Irelat.(%)): 443.5291 [M]+ (27). C₁₇H₁₃N₇O₂S₃. ¹H NMR (200 MHz, DMSO) δ 9.90 (s, 1H, NH), 8.10 (s, 1H, HC(5′)), 7.75-7.57 (m, 4H, HC(2″,3″,5″,6″)), 7.49 (s, 2H, NH₂), 2.68 (s, 3H, CH₃). ¹³C NMR (50 MHz, DMSO) δ 166.88 (C-2′), 159.39 (C-3), 157.45 (C-5), 144.66 (C-4″), 135.92 (C-4′), 133.01 (C-2″,6″), 132.45, 123.83 (C-5′), 119.42 (CN), 116.76 (C-3″, 5′), 101.60 (C-1″), 18.55 (CH₃).

### Example 53

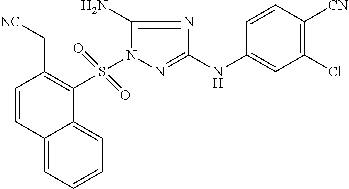 [SMILES: N#CCc1ccc2ccccc2c1S(=O)(=O)n1nc(Nc2ccc(C#N)c(Cl)c2)nc1N]

### 4-[[5-amino-1-[[7-(cyanomethyl)-1-naphthyl]sulfonyl]-1,2,4-triazol-3-yl]amino]-2-chloro-benzonitrile (Compound 53)

Yield 45%. Mass (EI), m/z (Irelat.(%)): 463.9005 [M]+ (43). C₂₁H₁₄ClN₇O₂S. ¹H NMR (200 MHz, DMSO) δ 10.07 (s, 1H, NH), 8.81 (s, 1H, HC(8′)), 8.49 (d, J=7.7 Hz, 1H, HC(2′)), 8.43 (d, J=8.0 Hz, 1H, HC(4′)), 8.19 (d, J=8.4 Hz, 1H, HC(5′)), 7-70-7.87 (m, 2H, HC(3′), HC(6′), HC(6″)), 7.66 (s, 2H, NH₂), 7.60 (d, J=1.9 Hz, 1H, HC(3″)), 7.49 (dd, J=8.7, 2.1 Hz, 1H, HC(5″)). ¹³C NMR (50 MHz, DMSO) δ 157.67 (C-3), 156.23 (C-5), 145.62 (C-4″), 136.43 (C-1′), 136.05 (C-2″), 135.24 (C-6″), 133.80 (C-8a′), 132.93 (C-2′), 131.31 (C-4a′), 128.90 (C-4′), 127.61 (C-6′), 126.80 (C-3′), 125.41 (C-5′), 125.02 (C-7′), 123.05 (C-8′), 118.63 (CN—CH₂), 116.65 (CN—C1″), 116.33 (C-5″), 116.22 (C-3″), 101.82 (C-1″), 23.31 (CH₂).

### Example 54

### Biological Studies

### Materials and Methods

In Vitro ADME assays: In vitro ADME studies were performed by BioDuro (San Diego, California, United States of America) and by Ecole Polytechnique Fédérale de Lausanne (EPFL) in Switzerland.

Bioanalytical Method for In Vitro ADME Studies: Test compounds were analyzed by reverse phase high-performance liquid chromatography (HPLC) with a 2.6μ C18 100A column sold under the tradename KINETEX® (3.0 mm×50 mm, Phenomenex (Torrance, California, United States of America)) using a Shimadzu (Columbia, Maryland, United States of America) LC-20AD system. The mobile phase consisted of solvent A (water with 0.1% formic acid) and solvent B (acetonitrile with 0.1% formic acid). The MS detection was performed by using an API 4000 Q trap system. The amount of parent compound was determined on the basis of the peak area ratio (compound area to internal standard area).

Kinetic Solubility: 396 μL of Universal Aqueous buffer (pH 7.4) was added to 4 μL of a 50 mM DMSO stock solution of each compound. Wells were agitated for 4 hours at 20° C. and then filtered. The compound was then diluted to serial concentrations with DMSO, followed by serial dilutions with ACN:H2O (1:1) prior to liquid chromatography-mass spectroscopy (LCMS) analysis. The calculated concentration (μM) of soluble compound was determined in reference to a standard curve.

Caco-2 Permeability: Caco-2 cells (ATCC®, Manassas, Virginia, United States of America) were grown on 24-well (pore size: 0.4 μm) polycarbonate filters. The monolayers were pre-incubated with pre-warmed HBSS (Hank's balanced salt solution) containing 2.5% HEPES buffer (pH 7.4) for 0.5 h at 37° C. After pre-incubation, the buffer was removed, and the experimental compounds were added to reach a final concentration of 10 μM. 2% bovine serum albumin (BSA) was added to the receiver buffer for the study. The total volume was 400 μL for the apical (A) side and 1200 μL for the basolateral (B) side. For apical to basolateral transport study (A-B), 100 μL each was collected from both sides for sample analysis at the start of the assay and then 200 μL was collected from the apical side at 90 minutes (end of the study). The same timepoints and amounts were used for the basolateral to apical transport study (B-A).

The apparent permeability coefficient (Papp) was calculated from the following equation.

$$Papp ( cm / sec ) = \frac{v \cdot \partial C / \partial t}{A \cdot C}$$

### Where:

v=Volume of the receiver cell
A=Exposed surface area (0.64 cm²)
C=Initial donor concentration
∂C/∂t=Change in receiver concentration over time

Human CYP Inhibition: Human liver microsome solution (0.2 mg/ml final concentration) (Sekisui Xenotech, Kansas City, Kansas, United States of America), along with substrate, was aliquoted into a 0.05 M phosphate buffer (pH=7.4) in 1.1 ml tubes. Study samples (containing either control inhibitor or test compound) were added into the tubes, vortexed gently and pre-incubated for 5 min at 37° C. 20 μL of NADPH solution was aliquoted into all tubes, then vortexed to start the reaction and to assure adequate mixture of the NADPH. After mixing, the tubes were incubated for 20 min at 37° C. in a shaking water bath and then quenched in 300 ml formic acid/acetonitrile solution. After quenching, the samples were vortexed vigorously for 1 min and centrifuged at 4,000 rpm for 15 min (4° C.). 100 μL of supernatant was transferred to 0.65 ml tubes for LCMS analysis by the bioanalytical method described earlier. The CYP450 substrates and control inhibitors for each enzyme was as follows: CYP1A2 (phenacetin, naphthoflavone), CYP2C9 (diclofenac, sulfaphenazole), CYP2C19 (omeprazole, tranylcypromine), CYP2D6 (dextromethorphan, quinidine), CYP3A4 (midazolam, ketoconazole).

Mouse/Human liver microsome stability: A liver microsome solution (197.5 μL, 1.27 mg/ml protein concentration) (Sekisui Xenotech, Kansas City, KS) was aliquoted into 1.1 ml tubes, to which 2.5 μL of positive control and compound stock solutions (100 μM in DMSO) were added. The tubes were vortexed gently, pre-incubated for 5 min at 37° C., then 50 μL of 5 mM NADPH or LM buffer (no NADPH buffer) was added into the tubes. For analysis, an aliquot of 15 μL was removed from each tube at 0, 5, 15, 30 and 60 min (without-NADPH reaction: 0, 30 and 60 min) and quenched with 300 μL of 25 ng/ml propranolol in acetonitrile. Samples were vigorously vortexed for 1 min and then centrifuged at 4,000 rpm for 15 min at 4° C. 100 μL of supernatant from each sample was transferred to 0.65 ml tubes for LCMS analysis. The amount of parent compound was determined on the basis of the peak area ratio (compound area to IS area) for each time point. Clearance rates were calculated by the equation:

$${CL}_{int} ( µL / min / mg protein ) = Ln ( 2 ) * 1000 / {T}_{1 / 2} / Protein Conc .$$

Protein Binding in Plasma: The donor side of dialysis inserts were filled with 200 μL plasma (human and mouse; source BioDuro, San Diego, California, United States of America) containing 5 μM drug and 0.5% of DMSO and the receiver side of the dialysis inserts was filled with 350 μL of PBS buffer (100 mM, pH 7.4). The prepared dialysis apparatus was placed in a shaker (37° C., 100 rpm) for 5 hours. Two tubes with plasma containing 5 μM experimental compound were also prepared for stability test: one tube was placed in the freezer (4° C.) for 5 hours and the other tube was placed in shaker (3° C., 100 rpm) for 5 hours. Samples were collected from the donor and receiver sides of each dialysis insert. The same volume of blank plasma was added to buffer samples and blank buffer to plasma samples to make sure all sample mixtures contain 50% plasma and 50% buffer. 50 μL of each sample was mixed with 300 μL of acetonitrile containing 25 ng/ml internal standard (propranolol). All samples were vortexed for 1 minute and then centrifuged at 4000 rpm, 4° C. for 15 min. 100 μL of the supernatant was transferred to 0.65 ml tube for LCMS analysis. The amount of compound was determined on the basis of the peak area ratio (compound area to internal standard area) for the two sides, and protein binding is determined using the following equations: % Bound=100×([Area Ratio of Donor]5h*5−[Area Ratio of Receiver]5h)/([Area Ratio of Donor]5h*5). The percentage remaining at 37° C. after 5 hours was calculated on the basis of the amount measured at 0° C. after 5 hours.

Mutagenicity evaluation: Escherichia coli PQ37 strain was grown overnight at 37° C., with shaking, in Luria Broth Base supplemented with 50 μg/mL Ampicillin. Bacteria were grown to mid-logarithmic phase and adjusted to an optical density of 0.4.

The second day, Luria Broth Base supplemented with 1.5% Agar was autoclaved for 15 minutes at 121° C. and then put into a water bath at 55° C. for 1 hour.

80 mL of warm agar was transferred into a plastic bottle (Milian, PETG 2019-0125) supplemented with 50 mg/mL ampicillin and 0.005% X-Gal (dissolved into DMF). 4 mL of E. coli PQ37 was added to the mixture and 70 mL of it was transferred into a square petri dish (Grenier Bio-One, Kremsmunster, Austria, 120×120 mm). Petri dish containing the mixture was dried 15 minutes at RT and once the mixture became solid, 6 mm absorbent disks were stuck on the plate.

5 μL of each compound were added on the disk with a pipet sold under the tradename PIPETMAN® P20 (Gilson S.A.S., Villiers-Le-Bel, France) and the petri dish was incubated overnight at 37° C.

The third day, zones of inhibition were recorded with a ruler and a picture of the plate was taken with a scanner (Canon 8800F, Cannon U.S.A., Inc., Melville, New York, United States of America).

Zone of inhibition was calculated by eye using a ruler and presence/absence of blue halo was reporter.

### In Vitro ADME/Tox Results

Compound 1 was initially chosen as a lead molecule for further study and its ADME properties were assessed. See Table 1A, below. Apart from relatively poor solubility (which does not appear to impact the mouse PK), compound 1 had good metabolic stability in mouse and human liver microsomes, low levels of CYP inhibition, high protein binding and no indication of efflux in Caco-2 cells. In addition to having positive ADMET properties, compound 1 also did not appear to be genotoxic in E. coli PQ37 in the SOS-chromotest at 10 mg/mL. The 99% toxic dose (TD99) in THP-1 and Huh-7D12 was >11 μg/ml (27.5 μM).

Compound 20 was selected as an additional lead molecule for further study and ADME properties were assessed for this compound as well. See Table 1B, below. Apart from relatively poor solubility, compound 20 had good metabolic stability, low levels of CYP inhibition, high protein binding and no indication of efflux in Caco-2 cells.

TABLE 1A
ADME Properties for Compound 1

| ADME property | Data |
|---|---|
| Solubility | <0.2 μM |
| CYP inhibition IC₅₀ | 1A2 (11.1 μM), 2C9 (26.5 μM), 2C19 |
| | (8.78 μM), 3A4 & 2D6 (>50 μM) |
| Mouse liver microsomes | t_{1/2} 66.6 min, CLᵢₙₜ 10.4 μL/min/mg |
| | protein, stable |
| Human liver microsomes | t_{1/2} 347 min, CLᵢₙₜ 2.0 μL/min/mg |
| | protein, stable |
| Mouse plasma protein binding | >99.9% |
| Human plasma protein binding | >99.9% |
| Caco-2 | Pₐₚₚ A-B = 0.138; B-A = 0.122 |
| | (×10−6 cm/s) Efflux ratio = 0.883 |

TABLE 1B
ADME Properties for Compound 20

| ADME property | Data |
|---|---|
| Solubility | <0.2 μM |
| CYP inhibition IC₅₀ | 1A2 & 2D6 (>50 μM), 2C9 (1.42 μM), 2C19 |
| | (10.6 μM), 3A4 (31.3 μM) |
| Mouse liver microsomes | t_{1/2} 124.4 min, CLᵢₙₜ 11.1 μL/min/mg protein, |
| | stable |
| Human liver microsomes | t_{1/2} 53.8 min, CLᵢₙₜ 25.8 μL/min/mg protein, |
| | stable |
| Mouse plasma protein | >99.9%, t_{1/2} > 372.8 min |
| binding and stability | |
| Human plasma protein | >99.9%, t_{1/2} > 372.8 min |
| binding and stability | |
| Caco-2 | Pₐₚₚ A-B = 0.4; B-A = 0.4 (×10⁻⁶ cm/s) Efflux |
| | ratio = 1 |

### Maximum Tolerated Dose (MTD) Study

An MTD dose study was performed in Federal Research Center “Fundamentals of Biotechnology RAS” in Moscow, Russia for compound 1. For this study 4 groups (120, 80, 60 and 40 mg/mouse of compound 1) of Balb/C male mice (5 animals per group) were observed over a 72 hr period to assess toxicity. Based on experimental data a median lethal dose (LD₅₀) was calculated for compound 1 with the confidence interval for the probability level of 0.95 by the method of Behrens-Kerber in the modification of the Van der Warda, using a proprietary computer program. The LD₅₀ was determined to be 78.60 mg/mouse or 3,930 mg/kg.

### In Vivo Pharmacokinetics

The pharmacokinetics of compound 1 was studied in white Balb/C male mice. 22 animals with an average body weight of 20 g were selected for the study. Before the study, the animals were kept on a standard vivarium ration with dry pelleted feed. All the test animals had free access to water but were deprived of food for 1 hour before administration of the compound. This regimen was continued for another hour after the administration.

Compound 1 in a dose of 250 mg/kg of body weight was administered as 0.5 ml of prepared suspension in 0.5% CMC by intragastric intubation. The animals were euthanized by decapitation for blood and brain sampling. Blood and brain samples (3 animals per time point) were taken at 0.5, 1, 2, 3, 5 and 7 hours after administration for pharmacokinetic evaluation. Blood was collected in heparinized tubes and centrifuged at 3500 RPM. Plasma was separated from formed elements and immediately frozen at −20° C. in freezer. This storage continued before transferring plasma for analysis. Serum from 6 untreated animals was used as a control and for equipment calibration with the compound. Mice brain was immediately frozen at −120° C.

Plasma separation: Samples of experimental blood (2.5 mL) from animals were centrifuged at 3500 rpm for 15 minutes.

Sample preparation: A mixture of 60 μL of plasma and 180 μL of acetonitrile (MeCN) by intensive shaking (Vortex) for 30 sec. Centrifugation 32000 g for 4 min. After centrifugation the 150 μL of organic layer was injected into the HPLC column.

Equipment: HPLC System Agilent 1290 Infinity, MS spectrometer Agilent 6460 (Agilent Technologies, Santa Clara, California, United States of America)

HPLC conditions: Column: Agilent Eclipse Plus C18 RRHD 1.8 um 2.1×50 mm (Agilent Technologies, Santa Clara, California, United States of America), Temperature: 40° C., Flow speed: 0.4 mL/min

Eluent: A: water, 0.1% formic acid; B: 85% acetonitrile in water, 0.1% formic acid

Gradient: 0.0-3.0 min: 60% B (separation), 3.0-4.0 min: 60->97% B (washing and regeneration), 4.0-6.0 min: 97% B, 6.0-6.1 min: 97->60% B, 6.1-9.0 min: 60% B, Volume of sample: 2 μL. Retention time in above conditions: 2.40 min.

MS conditions: ESI(+), MRM 400->209, Dwell time: 200 ms, Fragmentor: 135 V, Collision energy: 12, Cell Accelerator: 3 V, Gas Temp: 350° C., Gas Flow: 12 I/min, Nebulizer: 35 psi, Sheath gas Temp: 300° C., Capillary Voltage: 3500 V, Nozzle Voltage: 450 V; Diverter valve: 0.0-1.3 min: to waste; 1.3-3.0 min: to MS, data acquisition, 3.0-7.0 min: to MS, idle, 7.0- . . . min: to waste

Brain separation: Samples of experimental brain from animals were dispersed at 25K turn/min for 3 minutes and 100 mg of each sample was balanced to a sterile container.

Sample preparation: A mixture of 100 mg of brain and 100 μL of water was treated by zirconium balls in a homogonisator MagNA Lyser for 30 sec. 300 μL of MeCN was added and intensive shaking (Vortex) for 30 sec was performed. Centrifugation 32000 g for 4 min.

Equipment: HPLC System Agilent 1290 Infinity, MS spectrometer Agilent 6460 (Agilent Technologies, Santa Clara, California, United States of America).

HPLC conditions: Column: Agilent Eclipse Plus C18 RRHD 1.8 um 2.1×50 mm (Agilent Technologies, Santa Clara, California, United States of America), Temperature: 40° C., Flow speed: 0.4 mL/min.

Eluent: A: water, 0.1% formic acid; B: 85% acetonitrile in water, 0.1% formic acid.

Gradient: 0.0-3.0 min: 60% B (separation), 3.0-4.0 min: 60->97% B (washing and regeneration), 4.0-6.0 min: 97% B, 6.0-6.1 min: 97->60% B, 6.1-9.0 min: 60% B, Volume of sample: 2 μL.

Retention time in above conditions: 2.40 min

MS conditions: ESI(+), MRM 400->209, Dwell time: 200 ms, Fragmentor: 135 V, Collision energy: 12, Cell Accelerator: 3 V, Gas Temp: 350° C., Gas Flow: 12 l/min, Nebulizer: 35 psi, Sheath gas Temp: 300° C., Capillary Voltage: 3500 V, Nozzle Voltage: 450 V Diverter valve: 0.0-1.3 min: to waste; 1.3-3.0 min: to MS, data acquisition, 3.0-7.0 min: to MS, idle, 7.0- . . . min: to waste

in vivo PK results: Pharmacokinetic parameters were calculated with the ESTRIP computer program using model-independent method. The following parameters were calculated: maximum concentration (Cₘₐₓ)—maximal measured value; time to maximum concentration (Tₘₐₓ)—time of measuring of maximum concentration; area under pharmacokinetic curve (AUC₀₋ₜ)—within observation period (12 hours); calculated using trapezium method; area under pharmacokinetic curve (AUC_{0-∞})—from time zero to infinity; half-life (T½)—calculated using equation ln 2/kₒₗ; mean drug retention time in systemic bloodstream (MRT); relative absorption speed Cₘₐₓ/AUC₀₋ₜ; elimination constant—kₑₗ; volume of distribution—V_{d}; total clearance—Cl.

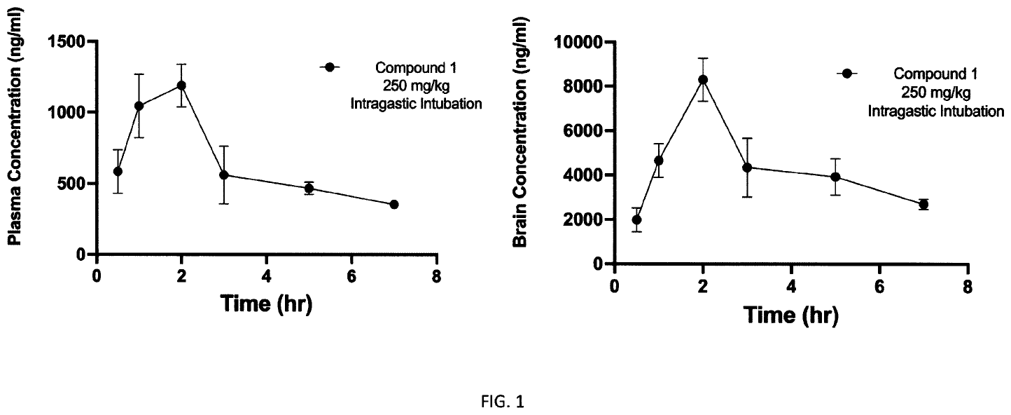

This preliminary single-dose PK study in mice suggested a T_{1/2} of 4.1h, Cₘₐₓ 1188 ng/ml. Preliminary data suggest very high concentrations of the compound in brain (1-5 μg/ml; 2.5-12.5 μM). Calculated parameters as well as the average plasma and brain concentration are shown in FIG. 1 and described in Table 2, below.

TABLE 2
In vivo PK for Compound 1.

| | Plasma Pharmacokinetic Parameters for Compound 1 | | | | | | | | |
|---|---|---|---|---|---|---|---|---|---|
| Mice | AUC₀₋₇ | AUC_{0-∞} | Cₘₐₓ | Tₘₐₓ | CL | Kₑₗ | T_{1/2} | MRT | Vd |
| group | (ng/ml*h) | (ng/ml*h) | (ng/ml) | (h) | (l/h) | (1/h) | (h) | (h) | (l) |
| 1 | 3714 | 6229 | 1047 | 2 | 0.803 | 0.1382 | 5.0 | 7.4 | 5.8 |
| 2 | 4361 | 6892 | 1170 | 2 | 0.725 | 0.1381 | 5.0 | 6.8 | 5.3 |
| 3 | 4079 | 5552 | 1346 | 2 | 0.901 | 0.2852 | 2.4 | 3.6 | 3.2 |
| mean | 4051 | 6224 | 1188 | 2 | 0.809 | 0.1872 | 4.1 | 5.9 | 4.8 |

### Inhibition and Toxicity Assays

Reagents: DEAE-dextran (Sigma-Aldrich, St. Louis, Missouri, United States of America) Cell titer Glo kit and Luciferase Assay System reagent are from Promega (Madison, Wisconsin, United States of America). HIV p24 (high sensitivity) A detection kit sold under the tradename ALPHALISA® Detection Kit from PerkinElmer (Waltham, Massachusetts, United States of America) was used. HIV I reverse transcriptase inhibition kits were from Roche (Indianapolis-Marion County, Indiana, United States of America).

Cells and virus culture: TZM-bl cells (42), HIV-1 IIIB virus (43-45) and HIV-1 IIIB (A17 variant) virus (46) as well are the NNRTI resistant mutants (strain NL4-3) (47) were obtained from the NIH HIV Reagent Program. H9 [derivative of HuT 78]cells (ATCC® HTB176™) were obtained from the American Type Culture Collection (Manassas, Virginia, United States of America). These viruses were grown in H9 cells to high titer, which was tested using a HIV p24 (high sensitivity) The detection kit used was sold under the tradename ALPHALISA® Detection Kit (PerkinElmer, Waltham, Massachusetts, United States of America). The viruses were concentrated using Lenti-X Concentrator (Takara Bio USA, Inc., Mountain View, California, United States of America). Infections of TZM-bl cells with either virus was facilitated with 15 μg/mL DEAE-dextran.

Compounds were serially diluted starting with 10 mM stocks in DMSO. DMSO concentrations were kept the same for all dilutions used within an assay (either 0.005% or 0.05% depending on highest concentration used). Duplicate 96-well plates were set up with 25 μL compound dilution, 25 μL virus, and 50 μL TZM-bl cells (2×10⁵/mL) with DEAE-dextran and incubated at 37° C. 5% CO₂ for 48 hr. All compounds were tested in triplicate, with single wells of efavirenz dilutions on each plate. All plates had 3-5 control wells without compound, 1 control well with no cells, and 1 control well with a highly toxic level of DMSO (9%). Inhibition assays were set up in clear tissue-culture treated plates with lysates moved to black assay plates or were set up in black tissue culture-treated plates. Medium was removed and the cells were lysed with Glo Lysis Buffer (Promega, Madison, Wisconsin, United States of America). Luciferase Assay System reagent (Promega, Madison, Wisconsin, United States of America) was added to cells and relative light units (RLU) was measured using an EnSpire instrument (PerkinElmer, Waltham, Massachusetts, United States of America). Toxicity assays were set up in clear tissue culture-treated plates. CellTiter-Blue Cell Viability Assay reagent (Promega) was added to the wells after 46 hr incubation, followed by 2 hr incubation. Sodium lauryl sulfate was added to 1.5% to stop the reaction and the plates were measured for fluorescence (560 nm_{Ex}/590 nm_{Em}) using an EnSpire instrument (PerkinElmer, Waltham, Massachusetts, United States of America).

A list of all of the IC₅₀'s from these in vitro inhibition experiments against the wild-type (IIIB) and Y181C/K103N (A17) IIIB variant are shown in Table 3, below.

TABLE 3
In vitro Inhibition data against HIV-1 IIIB and a related A17 (Y181C/K103N)
NNRTI-resistant double mutant in the TZM-bl cell line. All values have a
minimum of 6 replicates and IC₅₀s shown are ± SEM. These represent
a sampling of the total number of compounds tested.

| | | | Qualitative | |
|---|---|---|---|---|
| | | | Cell Toxicity | |
| | IC₅₀ | IC₅₀ | within | Quantitative |
| | (TZM, WT) | (TZM, A17) | inhibition | Cell Toxicity |
| Example | nM | nM | range | (nM) |
| Compound 1 | 9.33 ± 2.272 | >1000 | None | NT |
| Compound 2 | 1.11 ± 0.198 | 55.03 ± 4.006 | None | 5.78E+02 |
| Compound 3 | 1.57 ± 0.1896 | 572.6 ± 129.7 | None | NT |
| Compound 4 | 13.77 ± 2.641 | 286.7 ± 69.51 | None | NT |
| Compound 5 | 0.42 ± 0.0777 | 39.57 ± 18.22 | None | 1.71E+03 |
| Compound 6 | 70.24 ± 10.52 | 295.2 ± 56.45 | None | NT |
| Compound 7 | 19.21 ± 2.829 | 561.9 ± 5.11E+07 | None | NT |
| Compound 8 | 1.77 ± 0.3267 | 705.3 ± 1011 | None | NT |
| Compound 9 | 105.3 ± 24.33 | 708.6 ± 330.9 | None | NT |
| Compound 10 | 1.52 ± 0.3074 | 215.1 ± 57.44 | None | NT |
| Compound 11 | 0.98 ± 0.2454 | 174.9 ± 178.1 | Low | NT |
| Compound 12 | 0.8793 ± 0.072 | 305.7 ± 107.4 | None | NT |
| Compound 13 | 0.277 ± 0.0367 | 42.87 ± 34.68 | None | NT |
| Compound 14 | 0.6840 ± 0.077 | 37.25 ± 15.43 | None | 1.35E+03 |
| Compound 16 | 0.598 ± 0.105 | 153.4 ± 59.18 | None | NT |
| Compound 17 | 0.305 ± 0.053 | 904.9 ± 392.2 | None | NT |
| Compound 18 | 0.182 ± 0.029 | 238 ± 87.26 | None | NT |
| Compound 19 | 1.745 ± 0.445 | 193.4 ± 22.78 | None | NT |
| Compound 20 | 0.263 ± 0.050 | 0.6888 ± 0.1435 | None | 4.06E+03 |
| Compound 21 | 0.255 ± 0.035 | 5.424 ± 1.388 | None | 7.47E+02 |
| Compound 22 | 4.68 ± 0.572 | >1000 | None | NT |
| Compound 23 | 10.26 ± 2.013 | >1000 | None | NT |
| Compound 24 | 7.88 ± 0.6432 | NT | None | NT |
| Compound 25 | 4.54 ± 0.3298 | NT | None | NT |
| Compound 26 | 5.78 ± 0.4765 | NT | None | NT |
| Compound 27 | 34.31 ± 9.915 | >1000 | None | NT |
| Compound 28 | 83.69 ± 19.26 | NT | None | NT |
| Compound 29 | 14.32 ± 7.271 | NT | None | NT |
| Compound 30 | 21.54 ± 4.421 | NT | None | NT |
| Compound 31 | 19.38 ± 3.673 | NT | None | NT |
| Compound 32 | 14.46 ± 2.903 | >1000 | None | NT |
| Compound 33 | 240.9 ± 42.81 | NT | None | NT |
| Compound 34 | 92.56 ± 15.47 | NT | None | NT |
| Compound 35 | 14.32 ± 7.271 | NT | None | NT |
| Compound 36 | 44.12 ± 12.77 | >1000 | None | NT |
| Compound 37 | 10.15 ± 1.461 | >1000 | None | NT |
| Compound 38 | 177.3 ± 73.08 | >1000 | None | NT |
| Compound 39 | 575.2 ± 103.5 | >1000 | None | NT |
| Compound 40 | 15.09 ± 3.124 | >1000 | None | NT |
| Compound 41 | 10.13 ± 1.688 | 3683 ± 10866 | None | NT |
| Compound 42 | 28.12 ± 6.745 | >1000 | None | NT |
| Compound 43 | 33.67 ± 9.930 | >1000 | None | NT |
| Compound 44 | 164.8 ± 120.7 | >1000 | None | NT |
| Compound 45 | 36.14 ± 8.672 | >1000 | None | NT |
| Compound 46 | 394.1 ± 264 | >1000 | None | NT |
| Compound 47 | 153.1 ± 26.56 | >1000 | None | NT |
| Compound 48 | 13.98 ± 2.438 | >1000 | None | NT |
| Compound 49 | 107.6 ± 31.76 | >1000 | None | NT |
| Compound 50 | 19.45 ± 3.799 | >1000 | None | NT |
| Compound 51 | 0.223 ± 0.055 | >1000 | None | NT |
| Compound 52 | 2.14 ± 0.812 | >1000 | None | NT |
| Compound 53 | 5.29 ± 1.94 | >1000 | None | NT |

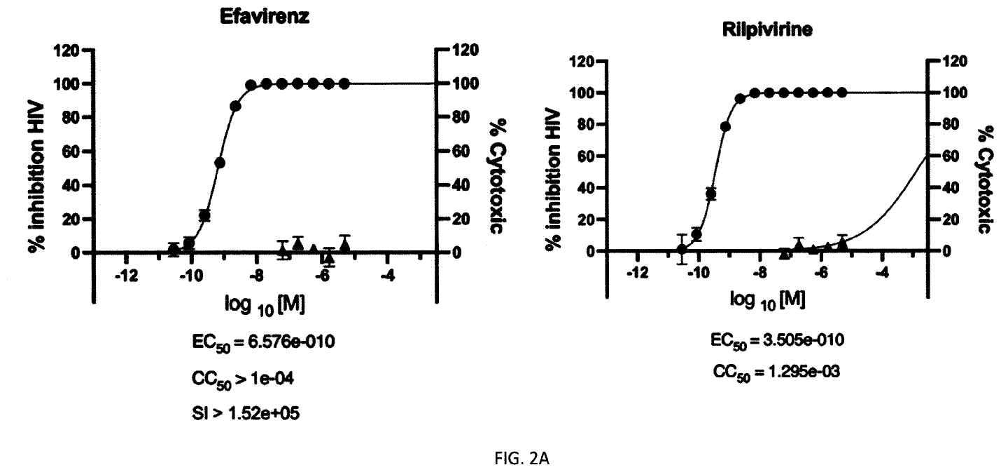

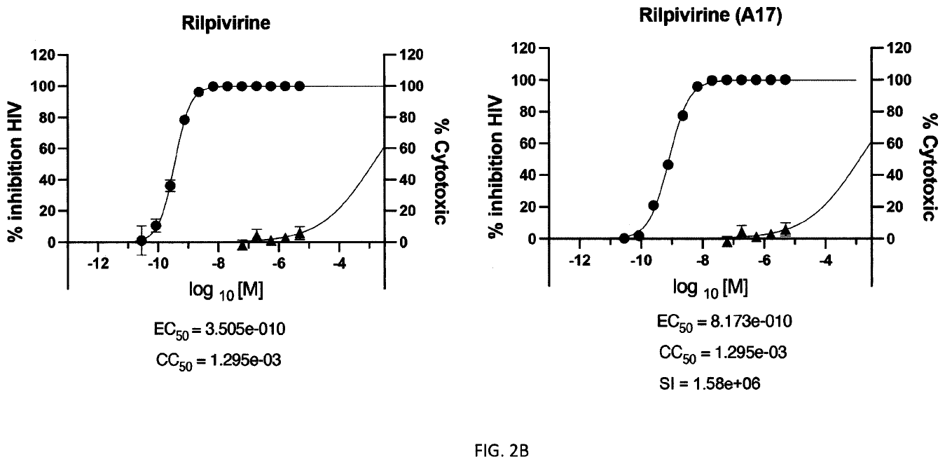

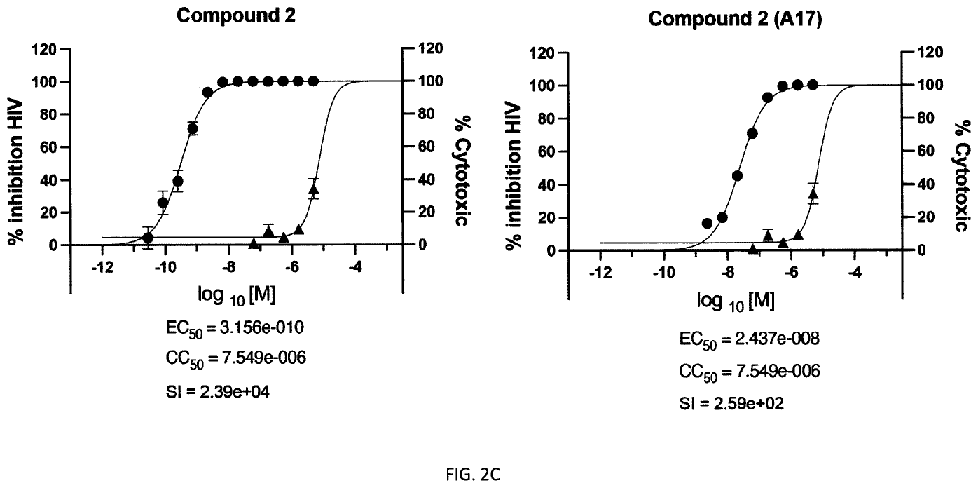

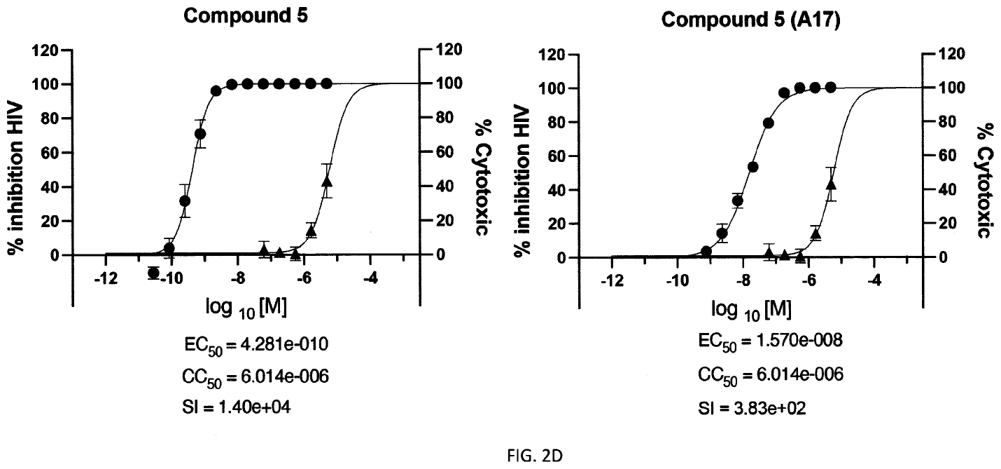

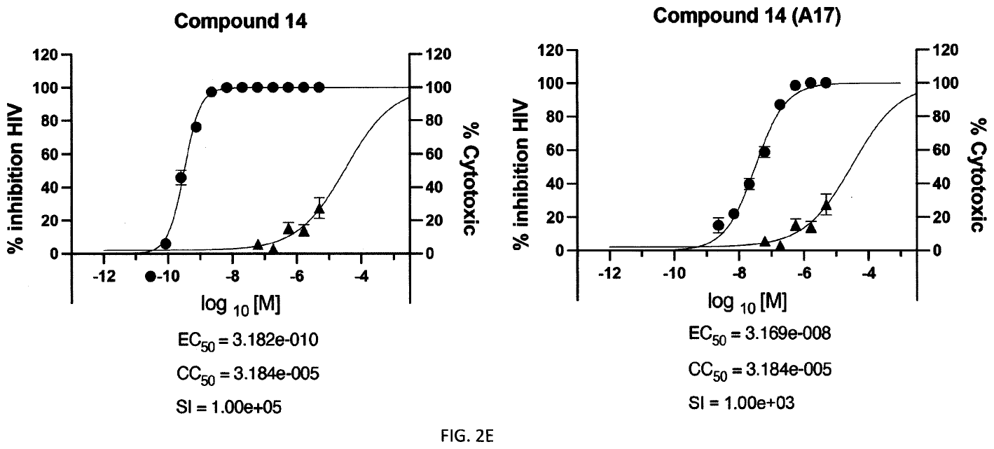

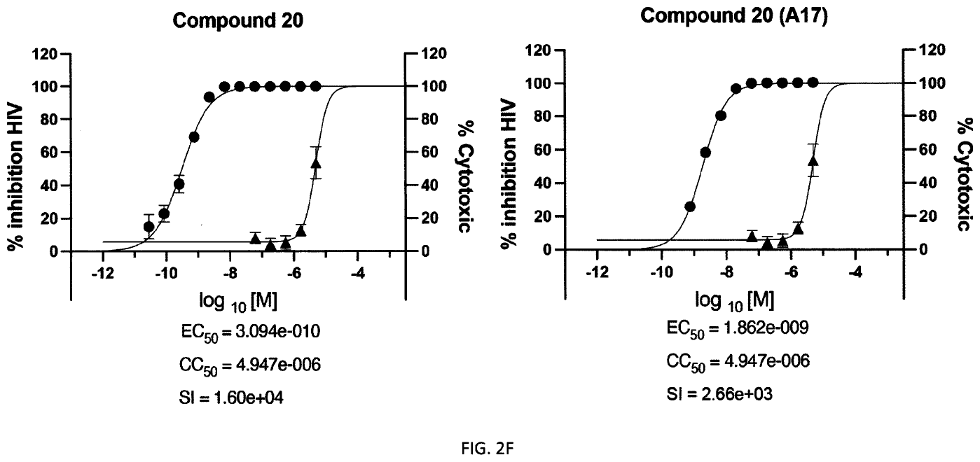

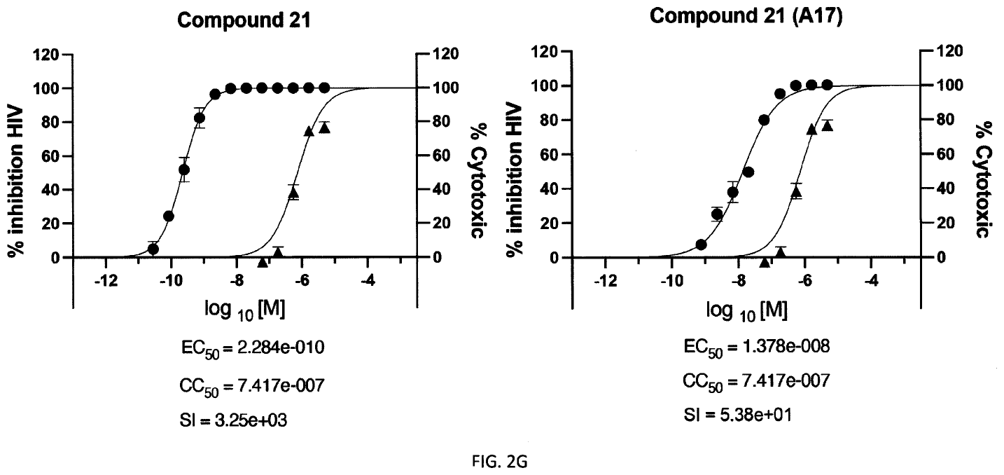

To confirm the activities of the most potent inhibitors, an extended range of concentrations was assessed against the wild-type and the A17 variant to calculated cytotoxicity in TZM-bl cells (0050). See FIGS. 2A-2G.

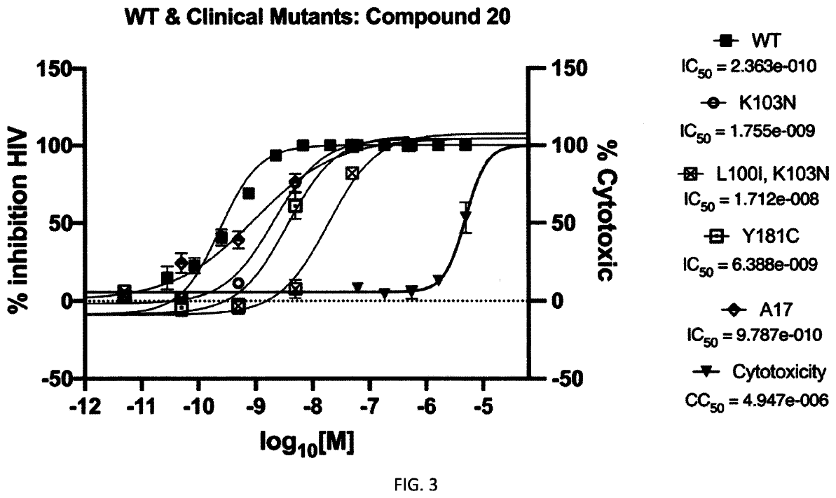

Compound 20 retained potent activity against the A17 variant, so it was assessed for activity in several NNRTI-resistant strains representing common single mutations Y181C and K103N. Results are shown in FIG. 3.

### Neuronal Cell Toxicity Studies

Primary cultures of mouse cortex and hippocampus: All culture work was done in accordance with NIH animal welfare guidelines and was approved by the University of North Carolina-Chapel Hill Institutional Animal Care and Use Committee. Timed gestational embryonic day 16 (E16) pregnant female CD1 mice (Charles River Laboratories, Wilmington, Massachusetts, United States of America) were anesthetized with the isoflurane drop method until breathing and heart stopped. A thoracotomy was then performed, the uterus removed, briefly rinsed in ice cold 70% ethanol, and rinsed twice in ice cold, sterile HEPES-buffered Hank's balanced salt solution (HBSS). The brain was dissected from each fetus, extensively washed, and cleaned of dura-arachnoid membrane and visible vessels. The cortex/hippocampus was dissected from each brain, minced, and transferred to a 15 ml tube containing 5 ml HBSS+2.4 U/ml dispase+2 U/ml DNase I and incubated for 25-30 min at 36° C. Tissue was triturated and pieces allowed to settle for 2 min. The suspended cells were transferred to a 50 ml culture tube containing 25 ml of Neurobasal Plus medium with added B27 Plus supplement, Glutamax, 5% fetal bovine serum and 20 μg/ml gentamicin. After several rounds of trituration in 2-3 ml calcium-magnesium free HBSS, the dissociated cells were seeded at a density of 12,000-20,000 cells/cm2 on poly-D-lysine-treated (0.1 mg/ml) coverslips. After 24 hours, cultures were transferred to Neurobasal Plus medium with added B27 Plus supplement and Glutamax. The resulting cultures were >95% neurons at day 4 after seeding.

Neuronal cell toxicity of primary mouse cultures (MAP-2 Staining): Compounds were added to primary cultures of mouse neurons at 20 days in vitro. A 10× stock dilution series of each drug was made up in artificial cerebrospinal fluid to be compatible with the medium but lacking protein supplements. A range of final concentrations from 0.1 to 10,000 nM was tested. 25 ul of each dilution was added to each well of a 48 well plate containing 225 ul of Neurobasal Plus medium with B27 Plus supplement. After 48 hrs the cells were fixed in methanol:acetic acid (97:3) and stained for microtubule associated protein-2 (MAP-2). Neuron number and morphology was then quantified with the aid of an image analysis software sold under the tradename METAMORPH® (Molecular Devices, San Jose, California, United States of America).

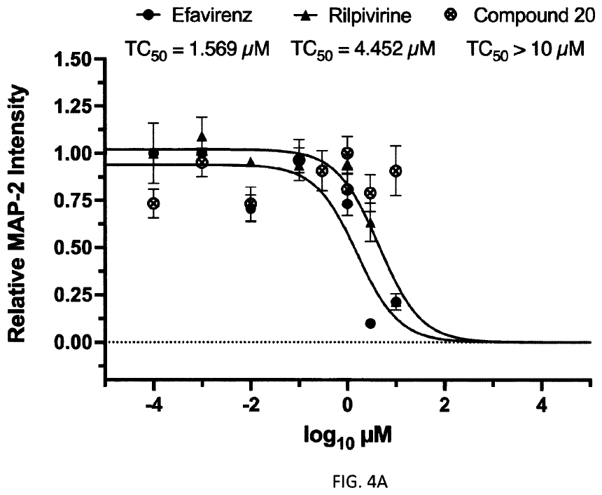

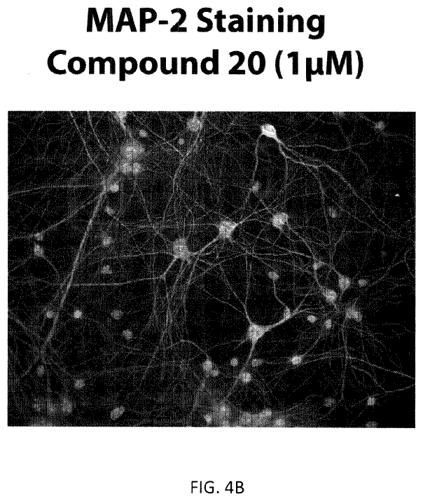

Using previously described methods (48), over the concentrations studied, no treatments resulted in complete loss of MAP-2 staining. The median toxic concentration (TC₅₀) value reflects the relative potency of the compound but not the total amount of damage and this relative toxicity is shown for compound 20 and two approved NNRTIs in FIG. 4A. FIG. 4B shows a representative image of stained cells.

### Calcium Imaging

The direct effects of the antiretroviral compounds were tested on primary rat and mouse neurons cultured on coverslips. Neurons at 14-18 days in vitro were loaded with the calcium indicator, Fluo-4 AM (2 μM, Molecular Probes, Inc., Eugene, Oregon, United States of America) in aCSF (aCSF: NaCl 137 10 mM, KCl 5.0 mM, CaCl2 2.3 mM, MgCl2 1.3 mM, glucose 20 mM). After 30 minutes of dye loading, the coverslip was transferred to a specialized stage for imaging. Cells were maintained in aCSF and time-lapse digital images were automatically captured on an Olympus IX71 microscope using software sold under the tradename METAMORPH® (Molecular Devices, San Jose, California, United States of America). Images were captured with a 40 msec exposure every 6 seconds for 6 minutes to assess acute effects and every minute for 60 minutes to assess delayed effects. Three pre-stimulation measurements were taken to establish basal levels of fluorescence at the beginning of each experiment. After collection of the third image, neurons the antiretroviral compound or vehicle diluted from the stock vial into aCSF was added to the neurons at the indicated final concentration. Baseline fluorescence was subtracted to correct for cell-to-cell differences in dye loading and intrinsic fluorescence. Changes in fluorescence at each time point were averaged across all cells from at least triplicate runs to provide an indication of the “typical” response. In some cases, individual cell response patterns are shown where the average masked important cell-specific profiles. All compounds were tested at 1 μM.

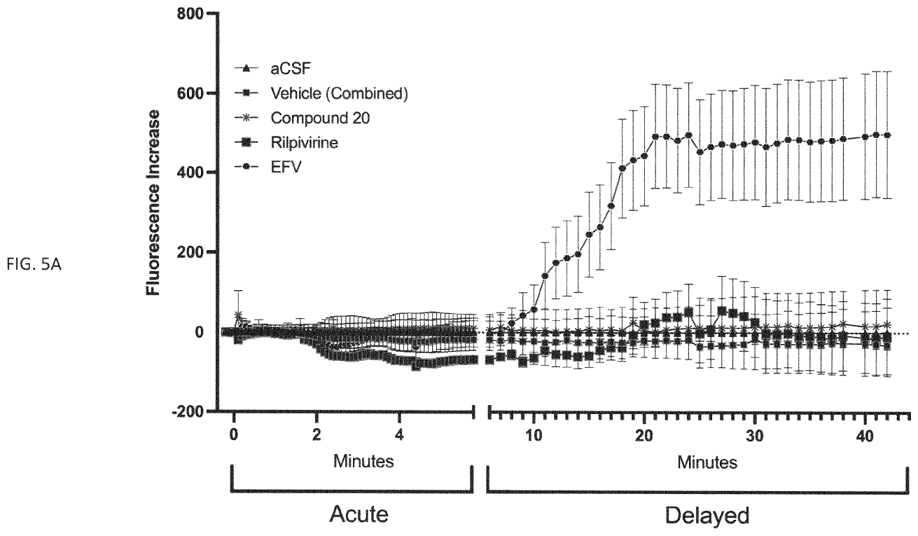

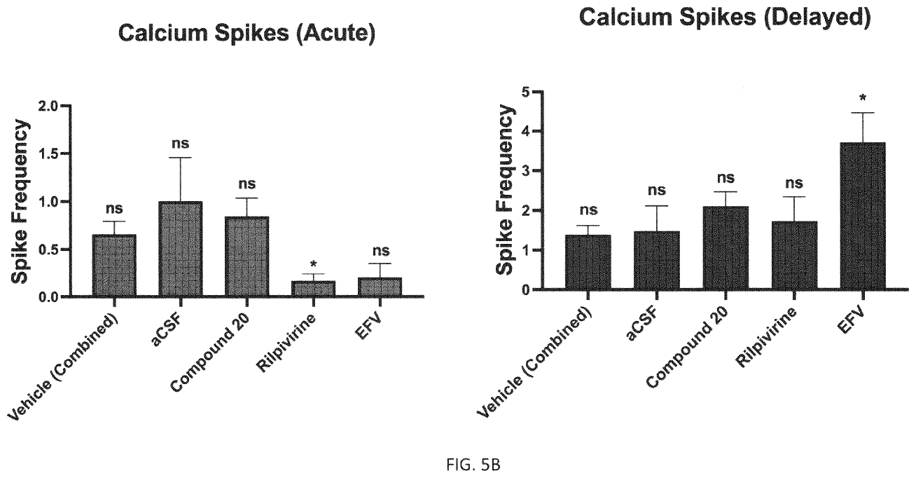

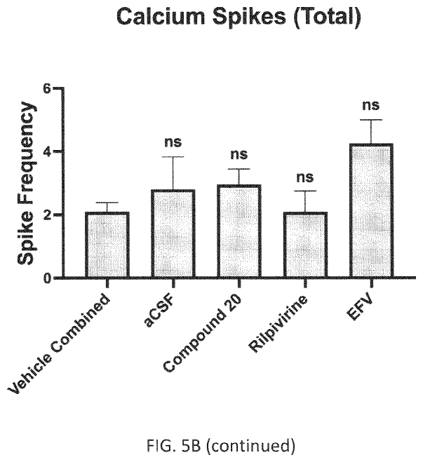

The calcium accumulation shows the average for all neurons and indicates whether the compounds activate calcium signaling (acute stage) and or provoke a delayed rise. Rilpivirine showed a decrease in acute spike frequency while EFV showed an increase in delayed spike frequency. EFV showed a trend towards a reduced acute signaling as well, but this was not statistically significant (p=0.2104). The normal range is typically thought to be about 2-4 calcium spikes per neuron, so the increases are relatively minor (49). From the patterns shown in the raw spike summaries (see FIG. 5B), this increase tends to be in the delayed phase, sometimes with a slight acute suppression, suggesting that the compounds are not totally benign, but the effects are small. Efavirenz showed the most dramatic increase in total calcium accumulation and this was more pronounced in the delayed phase. See FIG. 5A. (Figure. 5A). For reference, a toxic challenge that affects calcium regulation would provoke a delayed increase of about 800-1000 or more in fluorescence and anything under about 200 would be considered in the normal range. Typically, the controls would be +/−100 fluorescence units but these cultures had a little more basal activity than usual. Overall, only EFV seemed to have some toxic effects by this measure. The calcium spiking shows the calcium transients for individual neurons. It is possible for this indication of signaling activity to not necessarily translate to changes in the average calcium accumulation in the entire population.

### Reverse Transcriptase Inhibition Assays

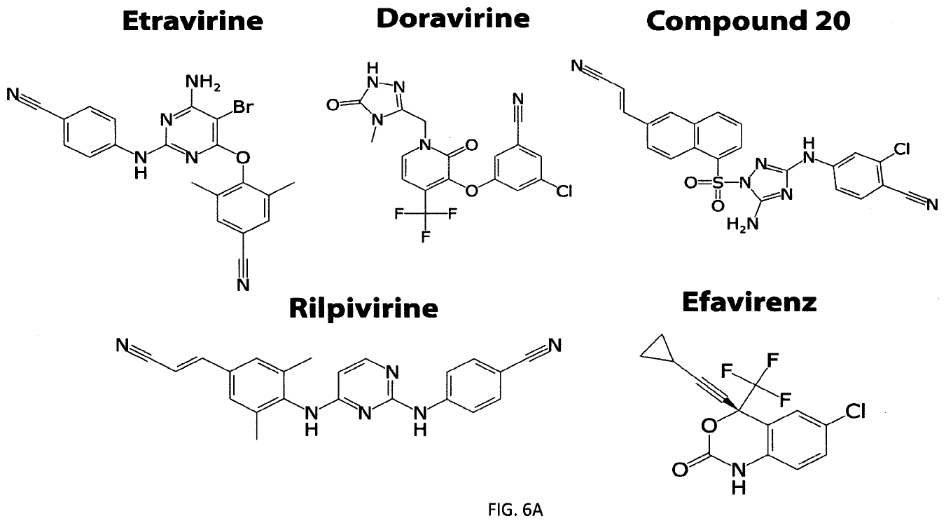

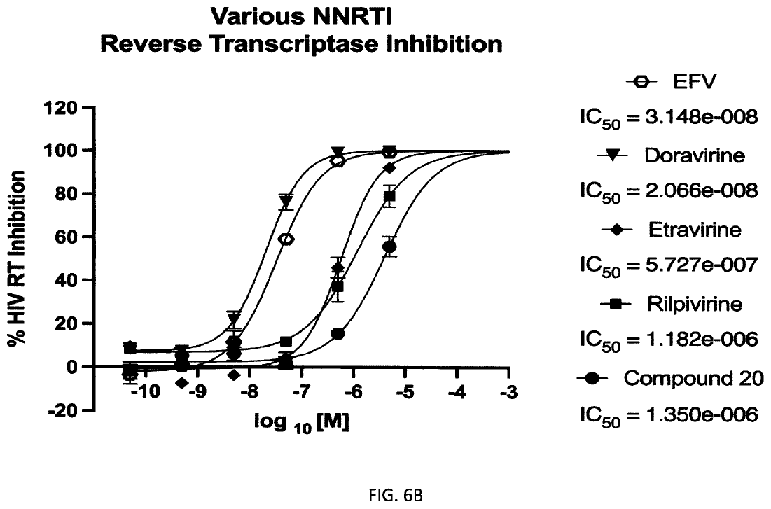

NNRTI assay methods: Inhibition of HIV I reverse transcriptase was assessed via a kit from Roche (Indianapolis-Marion County, Indiana, United States of America) using the manufacture's protocol (500 μM final reverse transcriptase reaction amount). In short, compounds (etavirine, doravirine, rilpivirine, efavirenz, and compound 20; see FIG. 6A) were serially diluted starting with 10 mM stocks in DMSO. DMSO concentrations were kept the same for all dilutions used within an assay (0.05%). Final compound concentrations ranged from 5000 nM-0.05 nM with 2-fold serial dilutions. 96-well plates were set up with 20 μL compound dilution, 20 μL reverse transcriptase, and 20 μL template/nucleotides and incubated at 37° C. 5% CO₂ for 1 hr in a reaction plate. 50 μL of each reaction was then transferred to a streptavidin conjugated 96-well plate and an ELISA assay was run based on manufacture's protocol. All compounds were tested in sextuplicate, with single wells of efavirenz dilutions on each plate. All plates had 8 control wells without compound, 2 control wells with no reverse transcriptase, and 2 control blank wells.

Results confirm HIV reverse transcriptase as a target of compound 20 and these results are shown in FIG. 6B for compound 20 as well as for several known NNRTIs. Interestingly, there is a disparity between the IC₅₀s for in-cell (TZM-bl) and reverse transcriptase HIV I inhibition for compound 20 of 1000-fold and this is surprisingly mirrored in rilpivirine. Without being bound to any one theory, this suggests that this difference is not unique for an NNRTI with reverse transcriptase as its likely primary target. The physiochemical properties are also shown for each compound as calculated by ChemAxon. In addition, each of the compounds that showed potent A17 inhibition in FIGS. 2A-2G were also tested for reverse transcriptase inhibition using this assay and the summary of these results are shown in Table 4, below.

TABLE 4
In vitro Inhibition (cell and reverse transcriptase) and toxicity
of compounds active against A17 NNTRI-resistant mutant.

| | IC₅₀ | IC₅₀ | CC₅₀ | EC₅₀ |
|---|---|---|---|---|
| | (TZM, WT) | (TZM, A17) | (TZM) | (RTase) |
| Name | nM | nM | nM | nM |
| Efavirenz | 0.658 | 17.01 | >1.00E+05 | 3.15E+01 |
| Rilpivirine | 0.351 | 0.817 | 1.12E+05 | 3.76E+02 |
| Compound 1 | 9.33 | >1000 | >1000 | 4.84E+03 |
| Compound 2 | 0.316 | 24.37 | 7.55E+03 | 5.78E+02 |
| Compound 5 | 0.428 | 15.70 | 6.01E+03 | 1.71E+03 |
| Compound 14 | 0.318 | 31.69 | 3.18E+04 | 1.35E+03 |
| Compound 20 | 0.309 | 45.35 | 4.95E+03 | 4.06E+03 |
| Compound 21 | 0.228 | 13.78 | 7.47E+02 | 1.09E+04 |

### Computational Docking

Structure-based drug discovery was performed by docking molecule designs in numerous HIV RT protein structures. Compounds were docked into the HIV I reverse transcriptase wild-type (PDB: 4G1Q) and K103N/Y181C double mutant (PDB: 4RW4) using Discovery Studio (Biovia, San Diego, California, United States of America) LibDock (rigid docking). The docking sphere was chosen based on the position of the crystalized ligands of rilpivirine and JLJ494 for wild-type and the K103N/Y181C double mutant, respectively. The docking protocols were all done with the default settings.

Initially, compound 1 was docked in a HIV RT (PDB 3MEC) crystal structure. The hit was shown to fit very well (Libdock score 140.36) and overlapped onto the docked rilpivirine (yellow; Libdock score 155.131) with similar predicted interactions. Docking of compound 20 also showed similar positioning as rilpivirine in both wild-type and the K103N/Y181C mutant with Libdock scores of 180.457 and 160.344, respectively.

Accordingly, a family of N-phenyl-1-(phenylsulfonyl)-1H-1,2,4-triazol-3-amine derivatives according to Formula (I) was prepared and shown to act as a new NNRTI scaffold.

Essentially all of the presently disclosed RT inhibitors typically have three parts, where the central core is an azaheterocycle and two other parts are substituted aryls. 95% of active compounds have a cyano group as a substituent. One phenyl moiety of the N-phenyl-1-(phenylsulfonyl)-1H-1,2,4-triazol-3-amine can be replaced, for example, with a heteroaryl, such as imidazothiophene, 1,3-thiazole and thiophenes. All these compounds show excellent activity against the whole cell wild-type virus. The replacement of the phenyl moiety to a heteroaryl moiety provides new possibilities for design of analogs with predefined biological and physicochemical properties.

One of the presently disclosed compounds, compound 20, is structurally distinct from other NNRTIs but has similar physicochemical properties. See Table 5, below. This molecule exhibited antiviral activity against wildtype (EC₅₀=0.22 nM) on a par with rilpivirine (EC₅₀=0.35 nM) with low cytotoxicity (CC₅₀=5.35 μM). See FIG. 2F. This compound also showed similar activity against the A17 double mutant (Y181C/K103N) with an EC₅₀=1.86 nM which is comparable with rilpivirine (EC₅₀=1.58 nM). See FIG. 2F. Interestingly, in the analysis of reverse transcriptase (RT) inhibition activity, compound 20 (IC₅₀=1350 nM) was on a par with rilpilvirine (IC₅₀=1182 nM). See FIG. 6B. Neuronal cell toxicity results are shown in FIG. 4A for compound 20 and two control NNRTIs (50). Compound 20 was selected as a lead molecule for further study and assessment of ADME properties. See Table 1B, above. Apart from the relatively poor solubility (intrinsic to HIV NNRTI compounds), this compound had good metabolic stability, low levels of CYP inhibition, high protein binding and no indication of efflux in Caco-2 cells.

TABLE 5
Physiochemical Property Comparison
of Compound 20 to Control NNTRIs.

| | | | | | | LogD |
|---|---|---|---|---|---|---|
| NNTRI | apKa1 | apKa2 | bpKa1 | bpKa2 | LogP | (pH 7.4) |
| Compound 20 | 8.54 | 17.55 | −0.65 | −5.51 | 4.33 | 4.3 |
| Doravirine | 9.66 | | −2.63 | −5.22 | 2.23 | 2.23 |
| Efavirenz | 12.52 | | −1.49 | | 4.46 | 4.46 |
| Etravirine | 10.99 | 19.91 | 3.49 | −3.02 | 5.54 | 5.54 |
| Rilpivirine | 11.43 | 16.33 | 4.44 | −2.53 | 5.47 | 5.47 |

### REFERENCES

The references listed below as well as all references cited in the specification are incorporated herein by reference to the extent that they supplement, explain, provide a background for or teach methodology, techniques and/or compositions employed herein. All cited patents and publications referred to in this application are herein expressly incorporated by reference.
1. Anon. UNAIDS. Global HIV & AIDS statistics—2020 fact sheet 2020.
2. WHO. HIV/AIDS 2020.
3. CDC. HIV Surveillance report 2018.
4. Murphy E L, Collier A C, Kalish L A, Assmann S F, Para M F, Flanigan T P, Kumar P N, Mintz L, Wallach F R, Nemo G J, Viral Activation Transfusion Study I. Highly active antiretroviral therapy decreases mortality and morbidity in patients with advanced HIV disease. Ann Intern Med. 2001; 135(1):17-26. Epub 2001/07/04. doi: 10.7326/0003-4819-135-1-200107030-00005. PubMed PMID: 11434728.
5. Wainberg M A. The Need for Development of New HIV-1 Reverse Transcriptase and Integrase Inhibitors in the Aftermath of Antiviral Drug Resistance. Scientifica (Cairo). 2012; 2012:238278. Epub 2012/01/01. doi: 10.6064/2012/238278. PubMed PMID: 24278679; PMCID: PMC3820659.
6. Shiau S, Abrams E J, Arpadi S M, Kuhn L. Early antiretroviral therapy in HIV-infected infants: can it lead to HIV remission?Lancet HIV. 2018; 5(5):e250-e8. Epub 2018/05/10. doi: 10.1016/S2352-3018(18)30012-2. PubMed PMID: 29739699; PMCID: PMC7487171.
7. Violari A, Cotton M F, Gibb D M, Babiker A G, Steyn J, Madhi S A, Jean-Philippe P, McIntyre J A, Team C S. Early antiretroviral therapy and mortality among HIV-infected infants. N Engl J Med. 2008; 359(21):2233-44. Epub 2008/11/21. doi: 10.1056/NEJMoa0800971. PubMed PMID: 19020325; PMCID: PMC2950021.
8. Esber A, Polyak C, Kiweewa F, Maswai J, Owuoth J, Maganga L, Adamu Y, Hickey P W, Ake J A, Crowell T A. Persistent Low-level Viremia Predicts Subsequent Virologic Failure: Is It Time to Change the Third 90?Clin Infect Dis. 2019; 69(5):805-12. Epub 2018/11/22. doi: 10.1093/cid/ciy989. PubMed PMID: 30462188.
9. Taiwo B, Gallien S, Aga E, Ribaudo H, Haubrich R, Kuritzkes D R, Eron J J, Jr. Antiretroviral drug resistance in HIV-1-infected patients experiencing persistent low-level viremia during first-line therapy. J Infect Dis. 2011; 204(4):515-20. Epub 2011/07/28. doi: 10.1093/infdis/jir353. PubMed PMID: 21791652; PMCID: PMC3203388.
10. Taramasso L, Magnasco L, Bruzzone B, Caligiuri P, Bozzi G, Mora S, Balletto E, Tatarelli P, Giacomini M, Di Biagio A. How relevant is the HIV low level viremia and how is its management changing in the era of modern ART?A large cohort analysis. J Clin Virol. 2020; 123:104255. Epub 2020/01/14. doi: 10.1016/j.jcv.2019.104255. PubMed PMID: 31927152.
11. Zhang T, Ding H, An M, Wang X, Tian W, Zhao B, Han X. Factors associated with high-risk low-level viremia leading to virologic failure: 16-year retrospective study of a Chinese antiretroviral therapy cohort. BMC Infect Dis. 2020; 20(1):147. Epub 2020/02/19. doi: 10.1186/s12879-020-4837-y. PubMed PMID: 32066392; PMCID: PMC7026956.
12. Ryscavage P, Kelly S, Li J Z, Harrigan P R, Taiwo B. Significance and clinical management of persistent low-level viremia and very-low-level viremia in HIV-1-infected patients. Antimicrob Agents Chemother. 2014; 58(7):3585-98. Epub 2014/04/16. doi: 10.1128/AAC.00076-14. PubMed PMID: 24733471; PMCID: PMC4068602.
13. Joya C, Won S H, Schofield C, Lalani T, Maves R C, Kronmann K, Deiss R, Okulicz J, Agan B K, Ganesan A. Persistent Low-level Viremia While on Antiretroviral Therapy Is an Independent Risk Factor for Virologic Failure. Clin Infect Dis. 2019; 69(12):2145-52. Epub 2019/02/21. doi: 10.1093/cid/ciz129. PubMed PMID: 30785191; PMCID: PMC6880328.
14. Laprise C, de Pokomandy A, Baril J G, Dufresne S, Trottier H. Virologic failure following persistent low-level viremia in a cohort of HIV-positive patients: results from 12 years of observation. Clin Infect Dis. 2013; 57(10):1489-96. Epub 2013/08/16. doi: 10.1093/cid/cit529. PubMed PMID: 23946221.
15. Tagnouokam-Ngoupo P A, Penda I C, Tchatchueng Mbougua J B, Tetang Ndiang S, Yuya Septoh F, Kenne A, Ngalle J E, Jakpou S, Ateba Ndongo F, Warszawski J, Faye A, Tejiokem M C, Group A N—P S. Virological failure and antiretroviral resistance among HIV-infected children after five years follow-up in the ANRS 12225-PEDIACAM cohort in Cameroon. PLoS One. 2021; 16(3):e0248642. Epub 2021/03/19. doi: 10.1371/journal.pone.0248642. PubMed PMID: 33735301; PMCID: PMC7971859.
16. Kao S W, Liu Z H, Wu T S, Ku S W, Tsai C L, Shie S S, Huang P Y, Wu Y M, Hsiao Y H, Chen N Y. Prevalence of drug resistance mutations in HIV-infected individuals with low-level viraemia under combination antiretroviral therapy: an observational study in a tertiary hospital in Northern Taiwan, 2017-19. J Antimicrob Chemother. 2021; 76(3):722-8. Epub 2020/12/18. doi: 10.1093/jac/dkaa510. PubMed PMID: 33331635.
17. Mziray S R, Kumburu H H, Assey H B, Sonda T B, Mahande M J, Msuya S E, Kiwelu I E. Patterns of acquired HIV-1 drug resistance mutations and predictors of virological failure in Moshi, Northern Tanzania. PLoS One. 2020; 15(9):e0232649. Epub 2020/09/29. doi: 10.1371/journal.pone.0232649. PubMed PMID: 32986709; PMCID: PMC7521739 competing interest: MJM is a member of the PLOS ONE Editorial Board. This does not alter our adherence to PLOS ONE policies on sharing data and materials. All other authors have declared that no competing interests exist.
18. Flexner C, Saag M. The antiretroviral drug pipeline: prospects and implications for future treatment research. Current opinion in HIV and AIDS. 2013; 8(6):572-8. doi: 10.1097/COH.0000000000000011. PubMed PMID: 24100879.
19. Tan I L, McArthur J C. HIV-associated neurological disorders: a guide to pharmacotherapy. CNS Drugs. 2012; 26(2):123-34. doi: 10.2165/11597770-000000000-00000. PubMed PMID: 22201342.
20. Sanmarti M, Ibanez L, Huertas S, Badenes D, Dalmau D, Slevin M, Krupinski J, Popa-Wagner A, Jaen A. HIV-associated neurocognitive disorders. J Mol Psychiatry. 2014; 2(1):2. doi: 10.1186/2049-9256-2-2. PubMed PMID: 25945248; PMCID: PMC4416263.
21. Bertrand L, Nair M, Toborek M. Solving the Blood-Brain Barrier Challenge for the Effective Treatment of HIV Replication in the Central Nervous System. Current pharmaceutical design. 2016. PubMed PMID: 27464720.
22. Cross H M, Combrinck M I, Joska J A. HIV-associated neurocognitive disorders: antiretroviral regimen, central nervous system penetration effectiveness, and cognitive outcomes. S Afr Med J. 2013; 103(10):758-62. Epub 2013/10/02. doi: 10.7196/samj.6677. PubMed PMID: 24079630.
23. Ellis R J, Letendre S, Vaida F, Haubrich R, Heaton R K, Sacktor N, Clifford D B, Best B M, May S, Umlauf A, Cherner M, Sanders C, Ballard C, Simpson D M, Jay C, McCutchan J A. Randomized trial of central nervous system-targeted antiretrovirals for HIV-associated neurocognitive disorder. Clin Infect Dis. 2014; 58(7):1015-22. Epub 2013/12/20. doi: 10.1093/cid/cit921. PubMed PMID: 24352352; PMCID: PMC3952601.
24. Di Yacovo M S, Molto J, Ferrer E, Curran A, Else L, Gisslen M, Clotet B, Tiraboschi J M, Niubo J, Vila A, Zetterberg H, Back D, Podzamczer D. Antiviral activity and CSF concentrations of 600/100 mg of darunavir/ritonavir once daily in HIV-1 patients with plasma viral suppression. J Antimicrob Chemother. 2015; 70(5):1513-6. Epub 2015/01/23. doi: 10.1093/jac/dku558. PubMed PMID: 25608583.
25. Van den Hof M, Blokhuis C, Cohen S, Scherpbier H J, Wit F, Pistorius MCM, Kootstra N A, Teunissen C E, Mathot RAA, Pajkrt D. CNS penetration of ART in HIV-infected children. J Antimicrob Chemother. 2018; 73(2):484-9. Epub 2017/11/11. doi: 10.1093/jac/dkx396. PubMed PMID: 29126299.
26. Bertrand L, Meroth F, Tournebize M, Leda A R, Sun E, Toborek M. Targeting the HIV-infected brain to improve ischemic stroke outcome. Nat Commun. 2019; 10(1):2009. Epub 2019/05/03. doi: 10.1038/s41467-019-10046-x. PubMed PMID: 31043599; PMCID: PMC6494822.
27. Apostolova N, Funes H A, Blas-Garcia A, Galindo M J, Alvarez A, Esplugues J V. Efavirenz and the CNS: what we already know and questions that need to be answered. The Journal of antimicrobial chemotherapy. 2015; 70(10):2693-708. doi: 10.1093/jac/dkv183. PubMed PMID: 26203180.
28. Decloedt E H, Rosenkranz B, Maartens G, Joska J. Central nervous system penetration of antiretroviral drugs: pharmacokinetic, pharmacodynamic and pharmacogenomic considerations. Clin Pharmacokinet. 2015; 54(6):581-98. Epub 2015/03/18. doi: 10.1007/s40262-015-0257-3. PubMed PMID: 25777740.
29. Shah A, Gangwani M R, Chaudhari N S, Glazyrin A, Bhat H K, Kumar A. Neurotoxicity in the Post-HAART Era: Caution for the Antiretroviral Therapeutics. Neurotox Res. 2016. doi: 10.1007/s12640-016-9646-0. PubMed PMID: 27364698.
30. Abers M S, Shandera W X, Kass J S. Neurological and psychiatric adverse effects of antiretroviral drugs. CNS Drugs. 2014; 28(2):131-45. doi: 10.1007/s40263-013-0132-4. PubMed PMID: 24362768.
31. Jin J, Grimmig B, Izzo J, Brown L A, Hudson C, Smith A J, Tan J, Bickford P C, Giunta B. HIV non-nucleoside reverse transcriptase inhibitor efavirenz reduces neural stem cell proliferation in vitro and in vivo. Cell Transplant. 2016. doi: 10.3727/096368916X691457. PubMed PMID: 27076280.
32. Kranick S M, Nath A. Neurologic complications of HIV-1 infection and its treatment in the era of antiretroviral therapy. Continuum (Minneap Minn). 2012; 18(6 Infectious Disease): 1319-37. doi: 10.1212/01.CON.0000423849.24900.ec. PubMed PMID: 23221843; PMCID: PMC3760534.
33. Funes H A, Blas-Garcia A, Esplugues J V, Apostolova N. Efavirenz alters mitochondrial respiratory function in cultured neuron and glial cell lines. The Journal of antimicrobial chemotherapy. 2015; 70(8):2249-54. doi: 10.1093/jac/dkv098. PubMed PMID: 25925594.
34. Apostolova N, Funes H A, Bias-Garcia A, Alegre F, Polo M, Esplugues J V. Involvement of nitric oxide in the mitochondrial action of efavirenz: a differential effect on neurons and glial cells. The Journal of infectious diseases. 2015; 211(12):1953-8. doi: 10.1093/infdis/jiu825. PubMed PMID: 25538272.
35. Polo M, Alegre F, Funes H A, Bias-Garcia A, Victor V M, Esplugues J V, Apostolova N. Mitochondrial (dys)function—a factor underlying the variability of efavirenz-induced hepatotoxicity?British journal of pharmacology. 2015; 172(7):1713-27. doi: 10.1111/bph.13018. PubMed PMID: 25411110; PMCID: PMC4376451.
36. Sluis-Cremer N. The emerging profile of cross-resistance among the nonnucleoside HIV-1 reverse transcriptase inhibitors. Viruses. 2014; 6(8):2960-73. doi: 10.3390/v6082960. PubMed PMID: 25089538; PMCID: PMC4147682.
37. Melikian G L, Rhee S Y, Varghese V, Porter D, White K, Taylor J, Towner W, Troia P, Burack J, Dejesus E, Robbins G K, Razzeca K, Kagan R, Liu T F, Fessel W J, Israelski D, Shafer R W. Non-nucleoside reverse transcriptase inhibitor (NNRTI) cross-resistance: implications for preclinical evaluation of novel NNRTIs and clinical genotypic resistance testing. The Journal of antimicrobial chemotherapy. 2014; 69(1):12-20. doi: 10.1093/jac/dkt316. PubMed PMID: 23934770; PMCID: PMC3861329.
38. Vanangamudi M, Kurup S, Namasivayam V. Non-nucleoside reverse transcriptase inhibitors (NNRTIs): a brief overview of clinically approved drugs and combination regimens. Curr Opin Pharmacol. 2020; 54:179-87. Epub 2020/11/18. doi: 10.1016/j.coph.2020.10.009. PubMed PMID: 33202360.
39. Kerr S G, Anderson K S. Pre-steady-state kinetic characterization of wild type and 3′-azido-3′-deoxythymidine (AZT) resistant human immunodeficiency virus type 1 reverse transcriptase: implication of RNA directed DNA polymerization in the mechanism of AZT resistance. Biochemistry. 1997; 36(46):14064-70. Epub 1997/12/31. doi: 10.1021/bi9713862. PubMed PMID: 9369478.
40. Usach I, Melis V, Peris J E. Non-nucleoside reverse transcriptase inhibitors: a review on pharmacokinetics, pharmacodynamics, safety and tolerability. J Int AIDS Soc. 2013; 16:1-14. Epub 2013/09/07. doi: 10.7448/IAS.16.1.18567. PubMed PMID: 24008177; PMCID: PMC3764307.
41. Svarovskaia E S, Cheslock S R, Zhang W H, Hu W S, Pathak V K. Retroviral mutation rates and reverse transcriptase fidelity. Front Biosci. 2003; 8:d117-34. Epub 2002/11/29. doi: 10.2741/957. PubMed PMID: 12456349.
42. Wei X, Decker J M, Liu H, Zhang Z, Arani R B, Kilby J M, Saag M S, Wu X, Shaw G M, Kappes J C. Emergence of resistant human immunodeficiency virus type 1 in patients receiving fusion inhibitor (T-20) monotherapy. Antimicrob Agents Chemother. 2002; 46(6):1896-905. Epub 2002/05/23. doi: 10.1128/aac.46.6.1896-1905.2002. PubMed PMID: 12019106; PMCID: PMC127242.
43. Popovic M, Read-Connole E, Gallo R C. T4 positive human neoplastic cell lines susceptible to and permissive for HTLV-Ill. Lancet. 1984; 2(8417-8418): 1472-3. Epub 1984/12/22. doi: 10.1016/s0140-6736(84)91666-0. PubMed PMID: 6151082.
44. Popovic M, Sarngadharan M G, Read E, Gallo R C. Detection, isolation, and continuous production of cytopathic retroviruses (HTLV-Ill) from patients with AIDS and pre-AIDS. Science. 1984; 224(4648):497-500. Epub 1984/05/04. doi: 10.1126/science.6200935. PubMed PMID: 6200935.
45. Ratner L, Haseltine W, Patarca R, Livak K J, Starcich B, Josephs S F, Doran E R, Rafalski J A, Whitehorn E A, Baumeister K, et al. Complete nucleotide sequence of the AIDS virus, HTLV-Ill. Nature. 1985; 313(6000):277-84. Epub 1985/01/24. doi: 10.1038/313277a0. PubMed PMID: 2578615.
46. Nunberg J H, Schleif W A, Boots E J, O'Brien J A, Quintero J C, Hoffman J M, Emini E A, Goldman M E. Viral resistance to human immunodeficiency virus type 1-specific pyridinone reverse transcriptase inhibitors. J Virol. 1991; 65(9):4887-92. Epub 1991/09/01. doi: 10.1128/JVI.65.9.4887-4892.1991. PubMed PMID: 1714522; PMCID: PMC248949.
47. Balamane M, Varghese V, Melikian G L, Fessel W J, Katzenstein D A, Shafer R W. Panel of prototypical recombinant infectious molecular clones resistant to nevirapine, efavirenz, etravirine, and rilpivirine. Antimicrob Agents Chemother. 2012; 56(8):4522-4. Epub 2012/06/06. doi: 10.1128/AAC.00648-12. PubMed PMID: 22664973; PMCID: PMC3421573.
48. Robertson K, Liner J, Meeker R B. Antiretroviral neurotoxicity. J Neurovirol. 2012; 18(5):388-99. Epub 2012/07/20. doi: 10.1007/s13365-012-0120-3. PubMed PMID: 22811264; PMCID: PMC3581315.
49. Busche M A, Eichhoff G, Adelsberger H, Abramowski D, Wiederhold K H, Haass C, Staufenbiel M, Konnerth A, Garaschuk O. Clusters of hyperactive neurons near amyloid plaques in a mouse model of Alzheimer's disease. Science. 2008; 321(5896):1686-9. Epub 2008/09/20. doi: 10.1126/science.1162844. PubMed PMID: 18802001.
50. Krieger I V, Freundlich J S, Gawandi V B, Roberts J P, Gawandi V B, Sun Q, Owen J L, Fraile M T, Huss S I, Lavandera J L, loerger T R, Sacchettini J C. Structure-guided discovery of phenyl-diketo acids as potent inhibitors of M. tuberculosis malate synthase. Chemistry & biology. 2012; 19(12):1556-67. PubMed PMID: 23261599.

It will be understood that various details of the presently disclosed subject matter may be changed without departing from the scope of the presently disclosed subject matter. Furthermore, the foregoing description is for the purpose of illustration only, and not for the purpose of limitation.

1. A compound having a structure of Formula (I): 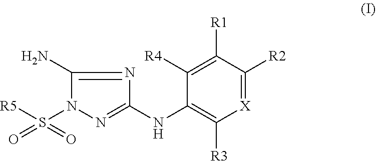
wherein
R1, R2, R3, and R4 are independently selected from the group consisting of H, CN, CH₂CN, CH═CHCN, F, Cl, and lower alkyl;
X is CR6 or N, wherein R6 is selected from the group consisting of H, CN, CH₂CN, CH═CHCN, COCH₃, C(CH₃)=CHCN, F, Cl, Br, lower alkyl, MeO, NMe₂, 4-F-Ph, and NMeCOCH═CH₂; and
R5 is a monovalent aryl group selected from the group consisting of: 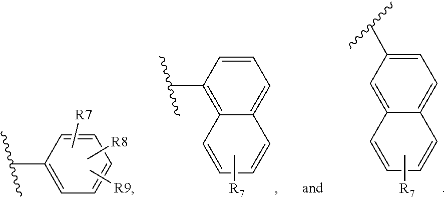
wherein R7, R8, and R9 are independently selected from the group consisting of H, CN, CH₂CN, CH═CHCN, COCH₃, C(CH₃)=CHCN, F, Cl, Br, lower alkyl, MeO, NMe₂, 4-F-Ph, and NMeCOCH═CH₂; or
wherein R5 is a monovalent heteroaryl group selected from the group consisting of: 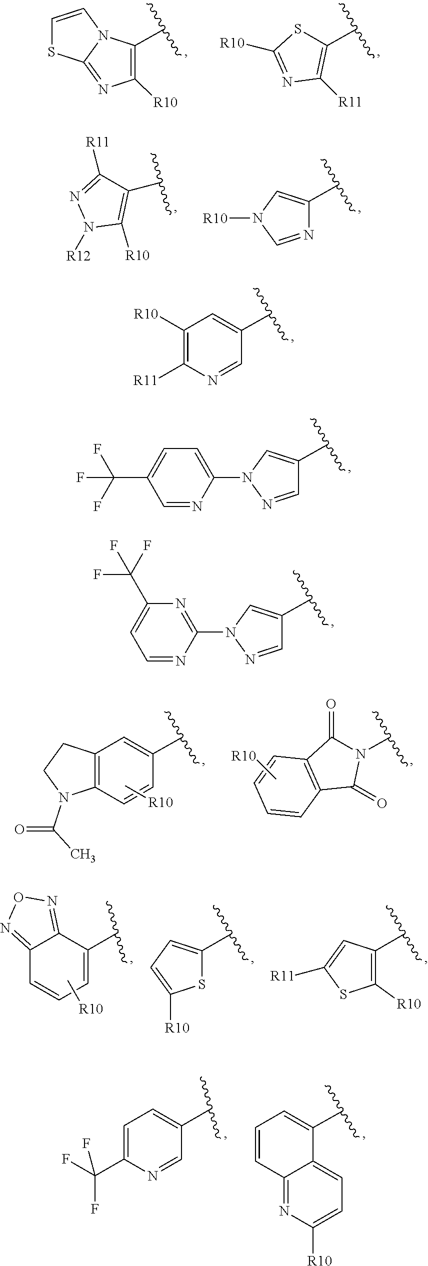 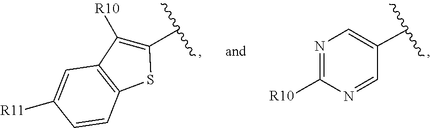
wherein R10, R11, and R12 are independently selected from the group consisting of H, F, Cl, Br, CN, lower alkyl, MeO, CF₃, and NHCOCH₃; or
a pharmaceutically acceptable salt thereof.

2. The compound of claim 1, wherein R5 is 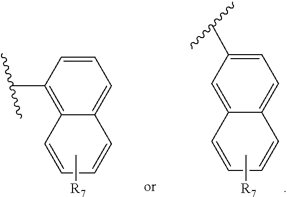
where R7 is selected from the group consisting of H, CN, CH₂CN, CH═CHCN, COCH₃, C(CH₃)=CHCN, F, Cl, Br, lower alkyl, MeO, NMe₂, 4-F-Ph, and NMeCOCH═CH₂.

3. The compound of claim 1, wherein R2 is selected from CN, CH₂CN, CH═CHCN, F, Cl, and lower alkyl.

4. The compound of claim 1, wherein R5 is: 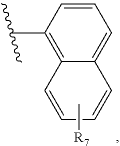
X is CR6, and R4 is H, and the compound of Formula (I) has a structure of: 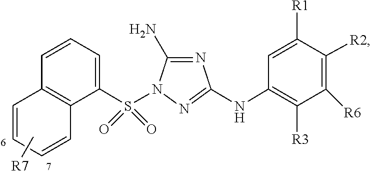
wherein R1, R2, and R3 are independently selected from the group consisting of H, CN, CH₂CN, CH═CHCN, F, Cl, and lower alkyl;
R6 is selected from the group consisting of H, CN, CH₂CN, CH═CHCN, COCH₃, C(CH₃)=CHCN, F, Cl, Br, lower alkyl, MeO, NMe₂, 4-F-Ph, and NMeCOCH═CH₂; and
R7 is selected from the group consisting of H, CN, CH₂CN, CH═CHCN, COCH₃, C(CH₃)=CHCN, F, Cl, Br, lower alkyl, MeO, NMe₂, 4-F-Ph, and NMeCOCH═CH₂;
optionally wherein R7 is attached to carbon 6 or carbon 7.

5. The compound of claim 4, wherein R1 and R3 are each H.

6. The compound of claim 5, wherein R2 is selected from CN, CH₂CN, and CH═CHCN and R6 is selected from H, CN, and C; optionally wherein R2 is CN and R6 is Cl, R2 is CN and R6 is CN, or R2 is CH₂CN and R6 is H.

7. The compound of claim 4, wherein R7 is CH═CHCN.

8. The compound of claim 1, wherein R5 is 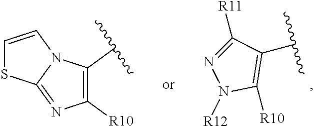
wherein R10, R11, and R12 are independently selected from the group consisting of H, F, Cl, Br, CN, lower alkyl, MeO, CF₃, and NHCOCH₃, optionally wherein R11 and R12 are each lower alkyl and R10 is selected from H and C.

9. The compound of claim 8, wherein X is CR6 and the compound has a structure of: 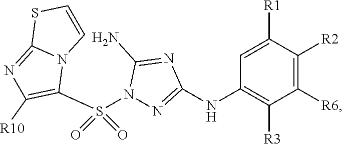
wherein R1, R2, and R3 are independently selected from the group consisting of H, CN, CH₂CN, CH═CHCN, F, Cl, and lower alkyl;
R6 is selected from the group consisting of H, CN, CH₂CN, CH═CHCN, COCH₃, C(CH₃)=CHCN, F, Cl, Br, lower alkyl, MeO, NMe₂, 4-F-Ph, and NMeCOCH═CH₂; and
R10 is selected from the group consisting of H, F, Cl, Br, CN, lower alkyl, MeO, CF₃, and NHCOCH₃, optionally wherein R10 is selected from H and C.

10. The compound of claim 9, wherein R1 and R3 are each H; R2 is selected from CN, CH₂CN, and CH═CHCN; and R6 is selected from H, CN, and C; optionally wherein R10 is Cl, further optionally wherein R2 is CN and R6 is H.

11. The compound of claim 1, wherein the compound is selected from the group consisting of:
5-[[5-amino-3-(4-cyanoanilino)-1,2,4-triazol-1-yl]sulfonyl]naphthalene-2-carbonitrile;
5-[[5-amino-3-(3-chloro-4-cyano-anilino)-1,2,4-triazol-1-yl]sulfonyl]-naphthalene-2-carbonitrile;
4-[[5-amino-1-(6-chloroimidazo[2,1-b]thiazol-5-yl)sulfonyl-1,2,4-triazol-3-yl]amino]-2-chloro-benzonitrile;
4-[[5-amino-1-[(6-cyano-1-naphthyl)sulfonyl]-1,2,4-triazol-3-yl]amino]-phthalonitrile;
4-[[5-amino-1-[[6-(cyanomethyl)-1-naphthyl]-sulfonyl]-1,2,4-triazol-3-yl]amino]benzonitrile;
4-[[5-amino-1-[[7-[2-cyanovinyl]-1-naphthyl]sulfonyl]-1,2,4-triazol-3-yl]amino]-2-chloro-benzonitrile;
4-[[5-amino-1-[[6-[2-cyanovinyl]-1-naphthyl]sulfonyl]-1,2,4-triazol-3-yl]amino]-2-chloro-benzonitrile; and
3-[5-[[5-amino-3-[4-(cyanomethyl)anilino]-1,2,4-triazol-1-yl]sulfonyl]-2-naphthyl]prop-2-enenitrile; or
a pharmaceutically acceptable salt thereof.

12. The compound of claim 1, wherein the compound is 4-[[5-amino-1-[[6-[2-cyanovinyl]-1-naphthyl]sulfonyl]-1,2,4-triazol-3-yl]amino]-2-chloro-benzonitrile; or a pharmaceutically acceptable salt thereof.

13. The compound of claim 1, wherein the compound is 4-[[5-amino-1-(6-chloroimidazo[2,1-b]thiazol-5-yl)sulfonyl-1,2,4-triazol-3-yl]amino]-2-chloro-benzonitrile, or a pharmaceutically acceptable salt thereof.

14. A pharmaceutical composition comprising a compound of claim 1 and a pharmaceutically acceptable carrier.

15. A method of treating or preventing a viral infection in a subject in need of thereof, wherein the method comprises administering to the subject a compound of claim 1, optionally wherein the subject is a human.

16. The method of claim 15, wherein the viral infection is a human immunodeficiency virus (HIV) infection.

17.-20. (canceled)

21. A method of treating or preventing a viral infection in a subject in need of thereof, wherein the method comprises administering to the subject a pharmaceutical composition of claim 14.

22. The method of claim 21, wherein the subject is a human.

23. The method of claim 22, wherein the viral infection is a human immunodeficiency virus (HIV) infection.
